(19) United States
(12) Patent Application Publication
(10) Pub. No.: US 2026/0234283 A1
(43) Pub. Date: Aug. 13, 2026

(54) ANTI-SIRP-alpha ANTIBODIES AND USES THEREOF

(71) Applicant: APEXIMMUNE THERAPEUTICS INC., Mountain View, CA (US)

(72) Inventors: Li-Fen LEE, Mountain View, CA (US); Kan LU, Mountain View, CA (US); Michihisa UMETANI, Mountain View, CA (US); Jessica YU, Mountain View, CA (US); Julie HUANG, Mountain View, CA (US); Ariel CHEN, Mountain View, CA (US)

(21) Appl. No.: 19/153,033

(22) PCT Filed: Feb. 5, 2024
(86) PCT No.: PCT/US2024/014437

§ 371 Date: Aug. 1, 2025

Related U.S. Application Data
(60) Provisional application No. 63/443,660, filed on Feb. 6, 2023.

(51) Int. Cl. C07K 16/28; A61K 39/00; A61K 45/06; A61K 47/68; A61P 35/00

(52) U.S. Cl. CPC C07K 16/2896; A61K 45/06; A61K 47/6849; A61P 35/00; A61K 2039/505; A61K 2039/545; C07K 2317/52; C07K 2317/565

(57) ABSTRACT

The present application provides anti-SIRPα constructs that bind to SIRPα (e.g, anti-SIRPα antibodies), nucleic acid molecules encoding amino acid sequences of the anti-SIRPα, vectors comprising the nucleic acid molecules, host cells comprising the vectors, methods of preparing the anti-SIRPα construct, pharmaceutical compositions comprising the anti-SIRPα construct, and methods of using the anti-SIRPα construct or compositions.

### CROSS-REFERENCE TO RELATED APPLICATIONS

This application claims the benefit of U.S. Provisional Patent Application No. 63/443,660, filed Feb. 6, 2023, the disclosure of which is incorporated by reference herein it its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14778-001-228_SEQ_LISTING.xml”, was created on Feb. 4, 2024, and is 130,005 bytes in size.

### 1. TECHNICAL FIELD

The present disclosure relates to anti-SIRPα antibodies and methods of use thereof.

### 2. BACKGROUND OF THE INVENTION

Cancer immunotherapy relies on the modulation of the immune system to increase recognition and response against tumor cells. Such modulation can be achieved by multiple mechanisms including the activation of co-stimulatory molecules present on immune cells or through the inhibition of co-inhibitory receptors. The activation of an immune response is a complex mechanism involving numerous cell populations like antigen-presenting cells important for the initiation of the antigen-specific response and immune cells responsible for tumor cell destruction. The mechanisms modulating the activity of these cells are numerous and represent target of choice in the context of cancer immunotherapy.

Signal regulatory protein a (SIRPα) is a transmembrane protein, the extracellular region of which comprises of 3 Ig-like domains and the cytoplasmic region contains immunoreceptor tyrosine-based inhibition motifs. SIRPα is primarily expressed within the myeloid cell lineage, such as macrophages, whereas it is barely expressed in T cells, B cells, and NK cells. The extracellular region of SIRPα interacts with the ligand CD47, which serves as a “don't eat me” signal, an inhibitory signal for phagocytosis of cancer cells by macrophages.

The CD47-SIRPα pathway acts as a key myeloid cell immune checkpoint and targeting the CD47/SIRPα axis represents a promising strategy to promote antitumor immunity. In contrast to the relatively limited distribution of SIRPα, CD47 is expressed on normal cell types, including red blood cells and platelets, as well as in cancer cells. Due to the broad expression of CD47, the antigen sink and hematologic toxicity, such as anemia and thrombocytopenia, are main issues for developing therapies targeting CD47. Considering the limited expression of SIRPα, targeting SIRPα is an alternative approach to block the CD47-SIRPα pathway. Given the apparent role of human SIRPα in modulating immune responses, therapeutic agents designed to antagonize SIRPα signaling hold great potential for the treatment of diseases that involve immune suppression.

### 3. BRIEF SUMMARY OF THE INVENTION

The present disclosure in one aspect provides an anti-SIRPα construct comprising an antibody moiety comprising a heavy chain variable region (V_{H}) and a light chain variable region (V_{L}).

In some embodiments, the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3, or a variant thereof comprising up to 5, 4, 3, 2, or 1 amino acid substitutions in the HC-CDRs; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11, or a variant thereof comprising up to 5, 4, 3, 2, or 1 amino acid substitutions in the HC-CDRs; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO:21, and a LC-CDR3 comprising the amino acid sequence of SEQ ID NO: 22, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94, or a variant thereof comprising up to 5, 4, 3, 2, or 1 amino acid substitutions in the HC-CDRs; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102, or a variant thereof comprising up to 5, 4, 3, 2, or 1 amino acid substitutions in the HC-CDRs; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105, or a variant thereof comprising up to 5, 4, 3, 2, or 1 amino acid substitutions in the LC-CDRs.

In some embodiments, the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6.

In some embodiments, the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14.

In some embodiments, the V_{H} comprises a HC-CDR1 comprising the amino acid sequence of SEQ ID NO: 62, a HC-CDR2 comprising the amino acid sequence of SEQ ID NO: 63 or 18, and a HC-CDR3 comprising the amino acid sequence of SEQ ID NO: 64 or 19; and the V_{L} comprises the LC-CDR1 comprising the amino acid sequence of SEQ ID NO: 65, a LC-CDR2 comprising the amino acid sequence of SEQ ID NO: 66, and a LC-CDR3 comprising the amino acid sequence of SEQ ID NO: 67.

In some embodiments, the V_{H} comprises a HC-CDR1 comprising the amino acid sequence of SEQ ID NO: 108, a HC-CDR2 comprising the amino acid sequence of SEQ ID NO: 109, and a HC-CDR3 comprising the amino acid sequence of SEQ ID NO: 80; and the V_{L} comprises a LC-CDR1 comprising the amino acid sequence of SEQ ID NO: 110, a LC-CDR2 comprising the amino acid sequence of SEQ ID NO:82, and a LC-CDR3 comprising the amino acid sequence of SEQ ID NO: 111.

In some embodiments, the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97.

In some embodiments, the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105.

In some embodiments, the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19; and the V_{L} comprises a LC-CDR1 comprising the amino acid sequence of SEQ ID NO: 20, a LC-CDR2 comprising the amino acid sequence of SEQ ID NO:21, and a LC-CDR3 comprising the amino acid sequence of SEQ ID NO: 22.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28.

In some embodiments, the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34.

In some embodiments, the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42.

In some embodiments, the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22.

In some embodiments, the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56.

In some embodiments, the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83.

In some embodiments, the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89.

In some embodiments, the present disclosure provides an anti-SIRPα construct comprising an antibody moiety that specifically binds to SIRPα, comprising:
1) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 7, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 8;
2) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 15, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 16;
3) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 23, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 24;
4) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 29, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 30;
5) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 35, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 36;
6) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 39, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 40;
7) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 43, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 44;
8) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 45, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 46;
9) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 49, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 50;
10) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 51, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 52;
11) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 54, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 55;
12) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 57, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 58;
13) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 60, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 61;
14) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 84, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 85;
15) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 90, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 91;
16) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 98, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 99; or
17) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 106, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 107.

In some embodiments according to any of the anti-SIRPα constructs described above, the V_{H} comprises an amino acid sequence of any one of SEQ ID NOs: 7, 15, 23, 29, 35, 39, 43, 45, 49, 51, 54, 57, 60, 84, 90, 98 and 106, or a variant comprising an amino acid sequence having at least about 80% sequence identity; and/or wherein the V_{L} comprises an amino acid sequence of any one of SEQ ID NOs: 8, 16, 24, 30, 36, 40, 44, 46, 50, 52, 55, 58, 61, 85, 91, 99 and 107, or a variant comprising an amino acid sequence having at least about 80% sequence identity.

In some embodiments according to any of the anti-SIRPα constructs described above, the V_{H} comprises the amino acid sequence of SEQ ID NO: 7, or a variant comprising an amino acid sequence having at least about 80% sequence identity; and the V_{L} comprises the amino acid sequence of SEQ ID NO: 8, or a variant comprising an amino acid sequence having at least about 80% sequence identity.

In some embodiments according to any of the anti-SIRPα constructs described above, the V_{H} comprises the amino acid sequence of SEQ ID NO: 15, or a variant comprising an amino acid sequence having at least about 80% sequence identity; and the V_{L} comprises the amino acid sequence of SEQ ID NO: 16, or a variant comprising an amino acid sequence having at least about 80% sequence identity.

In some embodiments according to any of the anti-SIRPα constructs described above, the V_{H} comprises the amino acid sequence of SEQ ID NO: 23, or a variant comprising an amino acid sequence having at least about 80% sequence identity; and the V_{L} comprises the amino acid sequence of SEQ ID NO: 24, or a variant comprising an amino acid sequence having at least about 80% sequence identity.

In some embodiments according to any of the anti-SIRPα constructs described above, the V_{H} comprises the amino acid sequence of SEQ ID NO: 29, or a variant comprising an amino acid sequence having at least about 80% sequence identity; and the V_{L} comprises the amino acid sequence of SEQ ID NO: 30, or a variant comprising an amino acid sequence having at least about 80% sequence identity.

In some embodiments according to any of the anti-SIRPα constructs described above, the V_{H} comprises the amino acid sequence of SEQ ID NO: 35, or a variant comprising an amino acid sequence having at least about 80% sequence identity; and the V_{L} comprises the amino acid sequence of SEQ ID NO: 36, or a variant comprising an amino acid sequence having at least about 80% sequence identity.

In some embodiments according to any of the anti-SIRPα constructs described above, the V_{H} comprises the amino acid sequence of SEQ ID NO: 39, or a variant comprising an amino acid sequence having at least about 80% sequence identity; and the V_{L} comprises the amino acid sequence of SEQ ID NO: 40, or a variant comprising an amino acid sequence having at least about 80% sequence identity.

In some embodiments according to any of the anti-SIRPα constructs described above, the V_{H} comprises the amino acid sequence of SEQ ID NO: 43, or a variant comprising an amino acid sequence having at least about 80% sequence identity; and the V_{L} comprises the amino acid sequence of SEQ ID NO: 44, or a variant comprising an amino acid sequence having at least about 80% sequence identity.

In some embodiments according to any of the anti-SIRPα constructs described above, the V_{H} comprises the amino acid sequence of SEQ ID NO: 45, or a variant comprising an amino acid sequence having at least about 80% sequence identity; and the V_{L} comprises the amino acid sequence of SEQ ID NO: 46, or a variant comprising an amino acid sequence having at least about 80% sequence identity.

In some embodiments according to any of the anti-SIRPα constructs described above, the V_{H} comprises the amino acid sequence of SEQ ID NO: 49, or a variant comprising an amino acid sequence having at least about 80% sequence identity; and the V_{L} comprises the amino acid sequence of SEQ ID NO: 50, or a variant comprising an amino acid sequence having at least about 80% sequence identity.

In some embodiments according to any of the anti-SIRPα constructs described above, the V_{H} comprises the amino acid sequence of SEQ ID NO: 51, or a variant comprising an amino acid sequence having at least about 80% sequence identity; and the V_{L} comprises the amino acid sequence of SEQ ID NO: 52, or a variant comprising an amino acid sequence having at least about 80% sequence identity.

In some embodiments according to any of the anti-SIRPα constructs described above, the V_{H} comprises the amino acid sequence of SEQ ID NO: 54, or a variant comprising an amino acid sequence having at least about 80% sequence identity; and the V_{L} comprises the amino acid sequence of SEQ ID NO: 55, or a variant comprising an amino acid sequence having at least about 80% sequence identity.

In some embodiments according to any of the anti-SIRPα constructs described above, the V_{H} comprises the amino acid sequence of SEQ ID NO: 57, or a variant comprising an amino acid sequence having at least about 80% sequence identity; and the V_{L} comprises the amino acid sequence of SEQ ID NO: 58, or a variant comprising an amino acid sequence having at least about 80% sequence identity.

In some embodiments according to any of the anti-SIRPα constructs described above, the V_{H} comprises the amino acid sequence of SEQ ID NO: 60, or a variant comprising an amino acid sequence having at least about 80% sequence identity; and the V_{L} comprises the amino acid sequence of SEQ ID NO: 61, or a variant comprising an amino acid sequence having at least about 80% sequence identity.

In some embodiments according to any of the anti-SIRPα constructs described above, the V_{H} comprises the amino acid sequence of SEQ ID NO: 84, or a variant comprising an amino acid sequence having at least about 80% sequence identity; and the V_{L} comprises the amino acid sequence of SEQ ID NO: 85, or a variant comprising an amino acid sequence having at least about 80% sequence identity.

In some embodiments according to any of the anti-SIRPα constructs described above, the V_{H} comprises the amino acid sequence of SEQ ID NO: 90, or a variant comprising an amino acid sequence having at least about 80% sequence identity; and the V_{L} comprises the amino acid sequence of SEQ ID NO: 91, or a variant comprising an amino acid sequence having at least about 80% sequence identity.

In some embodiments according to any of the anti-SIRPα constructs described above, the V_{H} comprises the amino acid sequence of SEQ ID NO: 98, or a variant comprising an amino acid sequence having at least about 80% sequence identity; and the V_{L} comprises the amino acid sequence of SEQ ID NO: 99, or a variant comprising an amino acid sequence having at least about 80% sequence identity.

In some embodiments according to any of the anti-SIRPα constructs described above, the V_{H} comprises the amino acid sequence of SEQ ID NO: 106, or a variant comprising an amino acid sequence having at least about 80% sequence identity; and the V_{L} comprises the amino acid sequence of SEQ ID NO: 107, or a variant comprising an amino acid sequence having at least about 80% sequence identity.

In some embodiments according to any of the anti-SIRPα constructs described above, the V_{H} comprises the amino acid sequence of SEQ ID NO: 7; and the V_{L} comprises the amino acid sequence of SEQ ID NO: 8.

In some embodiments according to any of the anti-SIRPα constructs described above, the V_{H} comprises the amino acid sequence of SEQ ID NO: 15; and the V_{L} comprises the amino acid sequence of SEQ ID NO: 16.

In some embodiments according to any of the anti-SIRPα constructs described above, the V_{H} comprises the amino acid sequence of SEQ ID NO: 23; and the V_{L} comprises the amino acid sequence of SEQ ID NO: 24.

In some embodiments according to any of the anti-SIRPα constructs described above, the V_{H} comprises the amino acid sequence of SEQ ID NO: 29; and the V_{L} comprises the amino acid sequence of SEQ ID NO: 30.

In some embodiments according to any of the anti-SIRPα constructs described above, the V_{H} comprises the amino acid sequence of SEQ ID NO: 35; and the V_{L} comprises the amino acid sequence of SEQ ID NO: 36.

In some embodiments according to any of the anti-SIRPα constructs described above, the V_{H} comprises the amino acid sequence of SEQ ID NO: 39; and the V_{L} comprises the amino acid sequence of SEQ ID NO: 40.

In some embodiments according to any of the anti-SIRPα constructs described above, the V_{H} comprises the amino acid sequence of SEQ ID NO: 43; and the V_{L} comprises the amino acid sequence of SEQ ID NO: 44.

In some embodiments according to any of the anti-SIRPα constructs described above, the V_{H} comprises the amino acid sequence of SEQ ID NO: 45; and the V_{L} comprises the amino acid sequence of SEQ ID NO: 46.

In some embodiments according to any of the anti-SIRPα constructs described above, the V_{H} comprises the amino acid sequence of SEQ ID NO: 49; and the V_{L} comprises the amino acid sequence of SEQ ID NO: 50.

In some embodiments according to any of the anti-SIRPα constructs described above, the V_{H} comprises the amino acid sequence of SEQ ID NO: 51; and the V_{L} comprises the amino acid sequence of SEQ ID NO: 52.

In some embodiments according to any of the anti-SIRPα constructs described above, the V_{H} comprises the amino acid sequence of SEQ ID NO: 54; and the V_{L} comprises the amino acid sequence of SEQ ID NO: 55.

In some embodiments according to any of the anti-SIRPα constructs described above, the V_{H} comprises the amino acid sequence of SEQ ID NO: 57; and the V_{L} comprises the amino acid sequence of SEQ ID NO: 58.

In some embodiments according to any of the anti-SIRPα constructs described above, the V_{H} comprises the amino acid sequence of SEQ ID NO: 60; and the V_{L} comprises the amino acid sequence of SEQ ID NO: 61.

In some embodiments according to any of the anti-SIRPα constructs described above, the V_{H} comprises the amino acid sequence of SEQ ID NO: 84; and the V_{L} comprises the amino acid sequence of SEQ ID NO: 85.

In some embodiments according to any of the anti-SIRPα constructs described above, the V_{H} comprises the amino acid sequence of SEQ ID NO: 90; and the V_{L} comprises the amino acid sequence of SEQ ID NO: 91.

In some embodiments according to any of the anti-SIRPα constructs described above, the V_{H} comprises the amino acid sequence of SEQ ID NO: 98; and the V_{L} comprises the amino acid sequence of SEQ ID NO: 99.

In some embodiments according to any of the anti-SIRPα constructs described above, the V_{H} comprises the amino acid sequence of SEQ ID NO: 106; and the V_{L} comprises the amino acid sequence of SEQ ID NO: 107.

In some embodiments according to any of the anti-SIRPα constructs described above, the antibody moiety is an antibody or an antigen-binding fragment thereof. In some embodiments, the antigen-binding fragment is selected from the group consisting of a full-length antibody, a bispecific antibody, a single-chain variable fragment (scFv) fragment, a Fab fragment, a Fab′ fragment, a F(ab′)₂, a variable fragment (Fv), a disulfide stabilized Fv fragment (dsFv), a (dsFv)₂, a Fv-Fc fusion, a scFv-Fc fusion, a scFv-Fv fusion, a diabody, a tribody, and a tetrabody. In some embodiments, the antibody moiety is a full-length antibody.

In some embodiments according to any of the anti-SIRPα constructs described above, the antibody moiety has an Fc fragment is selected from the group consisting of Fc fragments form IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment.

In some embodiments according to any of the anti-SIRPα constructs described above, the construct is a full-length antibody, a fusion protein, or an immunoconjugate.

In some embodiments according to any of the anti-SIRPα constructs described above, the construct is conjugated or recombinantly fused to a diagnostic agent, detectable agent or therapeutic agent. In some embodiments, the therapeutic agent is a chemotherapeutic agent, cytotoxin, or drug.

In some embodiments according to any of the anti-SIRPα constructs described above, the SIRPα is a human SIRPα.

The present disclosure in another aspect provides an anti-SIRPα construct competes for a binding epitope of SIRPα with any of the anti-SIRPα construct described above.

The present disclosure in another aspect provides a pharmaceutical composition comprising any of the anti-SIRPα constructs described above, and a pharmaceutical acceptable carrier.

The present disclosure in another aspect provides a nucleic acid encoding any of the anti-SIRPα constructs described above.

The present disclosure in another aspect provides a vector comprising any of the nucleic acids described above.

The present disclosure in another aspect provides a host cell comprising any of the nucleic acids or vectors described above.

The present disclosure in another aspect provides a method of producing an anti-SIRPα construct comprising: a) culturing any of the host cells described above under conditions effective to express the anti-SIRPα construct; and b) obtaining the expressed anti-SIRPα construct from the host cell.

The present disclosure in another aspect provides a method of treating a disease or condition in an individual, comprising administering to the individual an effective amount of any of the anti-SIRPα constructs or the pharmaceutical compositions described above. In some embodiments, the disease or condition is a tumor. In some embodiments, the tumor is cancer. In some embodiments, the tumor is a solid tumor. In some embodiments, the tumor is an advanced or malignant tumor. In some embodiments, the tumor has an increased expression level of SIRPα. In some embodiments, the tumor is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the disease or condition is a viral infection. In some embodiments, the expression level of SIRPα at an infected site is higher than that of an uninfected site.

In some embodiments according to any of the methods of treatment described above, the method further comprises administering to the individual a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the second agent comprises a cell comprising a chimeric antigen receptor that specifically binds to a tumor antigen. In some embodiments, the anti-SIRPα construct and the second agent are administered simultaneously or concurrently. In some embodiments, the anti-SIRPα construct and the second agent are administered sequentially. In some embodiments, the anti-SIRPα construct and/or the second agent are administered parentally.

In some embodiments according to any of the methods of treatment described above, the anti-SIRPα construct is administered to a tumor tissue or infection site directly.

In some embodiments according to any of the methods of treatment described above, the anti-SIRPα construct is administered at a dose of about 0.001 μg/kg to about 100 mg/kg.

In some embodiments according to any of the methods of treatment described above, the individual has an increased number of immune cells in a tumor tissue or at the infection site after administration of the anti-SIRPα construct. In some embodiments, the immune cells are T cells. In some embodiments, the T cells are activated T cells. In some embodiments, the number of immune cells in a tumor tissue or at the infection site is increased by at least about 5% after administration of the anti-SIRPα construct.

### 4. BRIEF DESCRIPTION OF FIGURES

FIGS. 1A-1E show the binding data of the presently disclosed anti-SIRPα antibodies. FIG. 1A depicts data of anti-SIRPα antibodies or control antibodies at a concentration of 10 μg/ml binding to 293T-hSIRPα cells stably expressing V1, V2 or V8 polymorphic variants of SIRPα, as detected by flow cytometry. FIG. 1B depicts dose titrations of anti-SIRPα antibodies or control antibody binding to 293T-hSIRPα V1 cells. FIG. 1C depicts dose titrations of anti-SIRPα antibodies or control antibody binding to 293T-hSIRPα V2 cells. FIG. 1D depicts dose titrations of anti-SIRPα antibodies or control antibody binding to 293T-hSIRPα V8 cells. FIG. 1E depicts dose titrations of anti-SIRPα antibodies or control antibody binding to hSIRPγ-expressing cells.

FIG. 2 shows data for selected anti-SIRPα antibodies demonstrating strong binding and affinity to recombinant SIRPα proteins by Biacore.

FIGS. 3A-3E show the blocking activities of the presently disclosed anti-SIRPα antibodies. FIG. 3A shows the results of a blocking assay between huCD47 and anti-SIRPα antibody for binding to 293-hSIRPα cells expressing polymorphic variants V1, V2 or V8. FIG. 3B shows dose titrations of anti-SIRPα antibodies or control antibody in blocking the interaction between huCD47 and SIRPα V1 stable expressed on 293T cells. FIG. 3C shows dose titrations of anti-SIRPα antibodies or control antibody in blocking the interaction between huCD47 and SIRPα V2 stable expressed on 293T cells. FIG. 3D shows dose titrations of anti-SIRPα antibodies or control antibody in blocking the interaction between huCD47 and SIRPα V8 stable expressed on 293T cells. FIG. 3E shows dose titrations of anti-SIRPα antibodies or control antibody in blocking the interaction between huCD47 and SIRPγ-expressing cells.

FIGS. 4A-4F show the impact of the presently disclosed anti-SIRPα antibodies on macrophage phagocytosis on tumor cells. FIG. 4A shows that a fixed concentration of anti-SIRPα antibodies potentiate macrophage phagocytosis of human DLD-1 tumor cells in the presence of a dose titration of cetuximab. FIG. 4B shows that anti-SIRPα antibodies dose-dependently induce macrophage phagocytosis of human DLD-1 tumor cells as a single agent. FIG. 4C shows that anti-SIRPα antibodies dose dependently potentiate macrophage phagocytosis of human DLD-1 tumor cells in the presence a fixed concentration of cetuximab. FIG. 4D shows that anti-SIRPα antibodies dose dependently potentiate phagocytosis of human DLD-1 tumor cells by SIRPα V1/V1, V1/V2, or V2/V2-bearing macrophages in the absence or presence of a fixed concentration of cetuximab. FIG. 4E shows that a fixed concentration of anti-SIRPα antibodies potentiate macrophage phagocytosis of Raji tumor cells in the presence of a dose titration of rituximab. FIG. 4F shows that anti-SIRPα antibodies dose dependently potentiate macrophage phagocytosis of human Raji tumor cells in the presence a fixed concentration of rituximab.

FIGS. 5A-5C show data of selected anti-SIRPα antibodies demonstrating no binding to red blood cells (FIG. 5A) or platelets (FIG. 5B), and no aggregation of red blood cells (FIG. 5C).

FIGS. 6A-6C show anti-tumor efficacy of selected anti-SIRPα antibodies on syngeneic MC38 tumors implanted in C57Bl/6 mice. FIG. 6A shows the data of mean tumor volumes over time as compared to an isotype control antibody. FIG. 6B shows individual tumor volumes for each treatment group on day 26 after tumor inoculation. FIG. 6C shows individual tumor growth curves for each treatment group over the duration of the study, with the number of tumor-free animals (complete response, CR) and the number of animals euthanized due to the maximum tumor volume being exceeded also listed.

### 5. DETAILED DESCRIPTION OF THE INVENTION

The present disclosure provides novel anti-SIRPα constructs that specifically bind to SIRPα (such as anti-SIRPα monoclonal or multispecific antibodies), methods of preparing the anti-SIRPα constructs, and methods of using the constructs (e.g., methods of treating a disease or condition).

### 5.1. Definitions

It is understood that embodiments of the application described terms of “comprising” herein include “consisting” and/or “consisting essentially of” embodiments.

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of a, 8, &, Y, and u heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelid single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and-binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above-cited references are set forth below in Table 1 as a comparison. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein. In some embodiments, the CDR sequences provided herein are based on IMGT definition. For example, the CDR sequences may be determined by the VBASE2 tool (http://www.vbase2.org/vbase2.php, see also Retter I, Althaus H H, Münch R, Müller W: VBASE2, an integrative V gene database. Nucleic Acids Res. 2005 Jan. 1; 33 (Database issue): D671-4, which is incorporated herein by reference in its entirety).

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, See Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two protein molecules is occupied by lysine, or if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the protein sequences SGTSTD and TGTSDA share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the stretch of residues C-terminal to a C_{H}2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, FcRN, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daeron, Annu. Rev. Immunol. 15:203-234 (1997). FcRN is critical to the recycling of an antibody to the blood allowing for increased serum half-life of the antibodies. FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731, which is incorporated herein by reference.

As used herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤about 10⁻⁵ M, ≤about 10⁻⁶ M, ≤about 10⁻⁷ M, ≤about 10⁻⁸ M, ≤about 10⁻⁹ M, ≤about 10⁻¹⁰ M, ≤about 10⁻¹¹ M, or ≤about 10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, BLI, RIA-, ECL-, IRMA-, EIA-, BIACORE™-tests and peptide scans.

An “isolated” or “purified” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

The term “immunoconjugate” includes reference to a covalent linkage of a therapeutic agent or a detectable label to an antibody such as an antibody moiety described herein. The linkage can be direct or indirect through a linker (such as a peptide linker).

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

A “therapeutically effective amount” of a substance/molecule of the application, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### 5.2. Anti-SIRPα Constructs

The present disclosure in one aspect provides anti-SIRPα constructs comprising an anti-SIRPα antibody moiety that specifically binds to SIRPα as described herein.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3, or a variant thereof comprising up to 5, 4, 3, 2, or 1 amino acid substitutions in the HC-CDRs; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 7; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 8.

In some embodiments, the V_{H} comprises the amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 8, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11, or a variant thereof comprising up to 5, 4, 3, 2, or 1 amino acid substitutions in the HC-CDRs; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 15; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 16.

In some embodiments, the V_{H} comprises the amino acid sequence of SEQ ID NO: 15, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 16, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 22, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 22.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 23; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 24.

In some embodiments, the V_{H} comprises the amino acid sequence of SEQ ID NO: 23, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 24, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprise a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 29; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 30.

In some embodiments, the V_{H} comprises the amino acid sequence of SEQ ID NO: 29, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 30, or a variant comprising the amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 35; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 36.

In some embodiments, the V_{H} comprises the amino acid sequence of SEQ ID NO: 35, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 36, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 39; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 40.

In some embodiments, the V_{H} comprises the amino acid sequence of SEQ ID NO: 39, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 40, or a variant comprising the amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 43 and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 44.

In some embodiments, the V_{H} comprises the amino acid sequence of SEQ ID NO: 43, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 44, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 45; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 46.

In some embodiments, the V_{H} comprises the amino acid sequence of SEQ ID NO: 45, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 46, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 49; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 50.

In some embodiments, the V_{H} comprises the amino acid sequence of SEQ ID NO: 49, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 50, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 51; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 52.

In some embodiments, the V_{H} comprises the amino acid sequence of SEQ ID NO: 51, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 52, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 54; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 55.

In some embodiments, the V_{H} comprises the amino acid sequence of SEQ ID NO: 54, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 55, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 57; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 58.

In some embodiments, the V_{H} comprises the amino acid sequence of SEQ ID NO: 57, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 58, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 60; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 61.

In some embodiments, the V_{H} comprises the amino acid sequence of SEQ ID NO: 60, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 62, a HC-CDR2 comprising the amino acid sequence of SEQ ID NO: 63 or 18, and a HC-CDR3 comprising the amino acid sequence of SEQ ID NO: 64 or 19; and the V_{L} comprises a LC-CDR1 comprising the amino acid sequence of SEQ ID NO: 65, a LC-CDR2 comprising the amino acid sequence of SEQ ID NO: 66, and a LC-CDR3 comprising the amino acid sequence of SEQ ID NO: 67.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 84; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 85.

In some embodiments, the V_{H} comprises the amino acid sequence of SEQ ID NO: 84, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 85, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 90; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 91.

In some embodiments, the V_{H} comprises the amino acid sequence of SEQ ID NO: 90, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 91, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94, or a variant thereof comprising up to 5, 4, 3, 2, or 1 amino acid substitutions in the HC-CDRs; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 98; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 99.

In some embodiments, the V_{H} comprises the amino acid sequence of SEQ ID NO: 98, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 99, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102, or a variant thereof comprising up to 5, 4, 3, 2, or 1 amino acid substitutions in the HC-CDRs; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-SIRPα antibody moiety is a humanized antibody derived from an anti-SIRPα antibody comprising a heavy chain variable region (V_{H}) and a light chain variable region (V_{L}), wherein the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105.

In some embodiments, the antibody moiety comprises a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 106; and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 107.

In some embodiments, the V_{H} comprises the amino acid sequence of SEQ ID NO: 106, or a variant comprising an amino acid sequence having at least about 80% (such as at least about any one of 80%, 85%, 90%, 95%, 96%, 97%, 98%, or 99%) sequence identity; and the V_{L} comprises the amino acid sequence of SEQ ID NO: 107, or a variant comprising an amino acid sequence having at least about 80% (such as at least about any one of 80%, 85%, 90%, 95%, 96%, 97%, 98%, or 99%) sequence identity.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 108, a HC-CDR2 comprising the amino acid sequence of SEQ ID NO: 109, and a HC-CDR3 comprising the amino acid sequence of SEQ ID NO: 80; and the V_{L} comprises a LC-CDR1 comprising the amino acid sequence of SEQ ID NO: 110, a LC-CDR2 comprising the amino acid sequence of SEQ ID NO: 82, and a LC-CDR3 comprising the amino acid sequence of SEQ ID NO: 111.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97.

In some embodiments, the anti-SIRPα construct comprises a heavy chain variable region (V_{H}) and a light chain variable region (V_{L}), wherein the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105.

In some embodiments, the V_{H} and/or the V_{L} further comprises a signaling peptide. In some embodiments, the signaling peptide is fused to the N-terminus of the V_{H} and/or the V_{L}.

In some embodiments, the construct comprises or is an antibody or an antigen-binding fragment thereof. In some embodiments, the antigen-binding fragment is selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)₂, a VHH, a Fv-Fc fusion, a scFv-Fc fusion, a scFv-Fv fusion, a diabody, a tribody, and a tetrabody.

In some embodiments, the anti-SIRPα antibody moiety is a full-length antibody.

In some embodiments, the anti-SIRPα antibody moiety is a scFv.

In some embodiments, the anti-SIRPα antibody moiety described above comprises an Fc fragment of an immunoglobulin selected from the group consisting of IgG, IgA, IgD, IgE, IgM, and combinations and hybrids thereof. In some embodiments, the anti-SIRPα antibody moiety or the full-length antibody described above comprises an Fc fragment of an immunoglobulin selected from the group consisting of IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment. In some embodiments the Fc fragment has been altered for increased serum half-life compared to the corresponding wildtype Fc fragment. In some embodiments the Fc fragment has been altered for decreased serum half-life compared to the corresponding wildtype Fc fragment.

In some embodiments, the antibody moiety comprises a humanized antibody of any of the antibody moiety described herein.

In some embodiments, the anti-SIRPα construct comprises or is an anti-SIRPα fusion protein.

In some embodiments, the anti-SIRPα construct comprises or is a multispecific anti-SIRPα construct (such as a bispecific antibody).

In some embodiments, the anti-SIRPα construct comprises or is an anti-SIRPα immunoconjugate.

In some embodiments, the SIRPα is a human SIRPα.

### 5.2.1. SIRPα

Signal regulatory protein a (SIRPα) is a regulatory membrane glycoprotein expressed mainly by myeloid cells, such as macrophages and dendritic cells. The gene encoding human SIRPα is polymorphic with the most prevalent variants being SIRPαV1, SIRPαV2, and SIRPαV8. SIRPα acts as a myeloid-lineage inhibitory receptor that restricts innate immunity through engagement of its cell surface ligand, CD47, to enact the “don't eat me” signal. This interaction negatively controls effector function of innate immune cells such as host cell phagocytosis. The SIRPα/CD47 pathway has emerged as an important innate immune checkpoint that enables cancer cell escape from macrophage phagocytosis.

In some embodiments, the SIRPα comprises the amino acid sequence set forth in SEQ ID NO: 77. In some embodiments, the presently disclosed anti-SIRPα construct binds to a portion of SIRPα

### 5.2.2. Antibody Affinity

Binding specificity of the antibody moieties can be determined experimentally by methods known in the art. Such methods comprise, but are not limited to Western blots, ELISA-, RIA-, ECL-, IRMA-, EIA-, BLI, BIACORE™-tests, flow cytometry and peptide scans.

In some embodiments, the K_{D} of the binding between the antibody moiety and SIRPα is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the Kp of the binding between the antibody moiety and SIRPα is stronger than about any one of 10⁻⁷ M, 10⁻⁸ M, 10⁻⁹ M, 10⁻¹⁰ M, 10⁻¹¹ M, or 10⁻¹² M. In some embodiments, the SIRPα is a human SIRPα.

In some embodiments, the Kₒₙ of the binding between the antibody moiety and SIRPα is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and SIRPα is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and SIRPα is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹. In some embodiments, SIRPα is human SIRPα.

In some embodiments, the K_{off} of the binding between the antibody moiety and SIRPα is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and SIRPα is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, SIRPα is human SIRPα.

In some embodiments, the binding affinity of the anti-SIRPα antibody moiety or anti-SIRPαconstruct are higher (for example, has a smaller K_{D} value) than an existing anti-SIRPα antibody (e.g., anti-human SIRPα antibody).

### 5.2.3. Chimeric or Humanized Antibodies

In some embodiments, the anti-SIRPα antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, the anti-SIRPα antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of mouse derived antibodies is a common and routinely used art. It is therefore understood that a humanized format of any and all of the anti-SIRPα antibodies disclosed in Sequence Table can be used in a preclinical or clinical setting. In cases where a humanized format of any of the referenced anti-SIRPα antibodies or their antigen-binding regions thereof is used in such a preclinical or clinical setting, the then humanized format is expected to bear the same or similar biological activities and profiles as the original non-humanized format.

### 5.2.4. Human Antibodies

In some embodiments, the anti-SIRPα antibody moiety is a human antibody (known as human domain antibody, or human DAb). Human antibodies can be produced using various techniques known in the art. Human antibodies are described generally in van Dijk and van de Winkel, Curr. Opin. Pharmacol. 5:368-74 (2001), Lonberg, Curr. Opin. Immunol. 20:450-459 (2008), and Chen, Mol. Immunol. 47 (4): 912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### 5.2.5. Library-Derived Antibodies

The anti-SIRPα antibody moieties described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, NJ, 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004). Methods for constructing single-domain antibody libraries have been described, for example, See U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### a) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity or molecular behavior.

Another method to introduce diversity involves HVR-directed approaches, in which several HVR residues (e.g., 4-6 residues at a time) are randomized. HVR residues involved in antigen binding may be specifically identified, e.g., using alanine or histidine scanning mutagenesis or modeling. HC-CDR3 and LC-CDR3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties for the antibody.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In some embodiments, the anti-SIRPα antibody moiety is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the anti-SIRPα antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

In some embodiments, the anti-SIRPα antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In some embodiments, the anti-SIRPα antibody moiety comprises an Fc fragment.

The term “Fc region,” “Fc domain,” “Fc fragment” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, MD, 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (See Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the IgG1 Fc fragment comprises a L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### e) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### 5.3. Anti-SIRPα Fusion Proteins

The anti-SIRPα constructs in some embodiments comprise an anti-SIRPα antibody moiety (e.g., an anti-SIRPα scFv) and a second moiety.

In some embodiments, the second moiety comprises a half-life extending moiety. In some embodiments, the half-life extending moiety is an albumin binding moiety (e.g., an albumin binding antibody moiety). In some embodiments, the anti-SIRPα antibody moiety and the half-life extending moiety is linked via a linker (such as any of the linkers described in the “Linkers” section).

### 5.3.1. Anti-SIRPα Immunoconjugates

In some embodiments, the anti-SIRPα construct described herein further comprises a second moiety. In some embodiments, the second moiety comprises a therapeutic agent. In some embodiments, the second moiety comprises a label. In some embodiments, the anti-SIRPα antibody moiety and the second moiety is linked via a linker (such as any of the linkers described in the “Linkers” section).

In some embodiments, the second agent is a cytotoxic agent. In some embodiments, the cytotoxic agent is a chemotherapeutic agent. In some embodiments, the cytotoxic agent is a growth inhibitory agent. In some embodiments, the cytotoxic agent is a toxin (e.g., protein toxins, enzymatically active toxins of bacterial, fungal, plant, or animal origin, or fragments thereof). In some embodiments, the cytotoxic agent is a radioactive isotype (i.e., a radio-conjugate).

Immunoconjugates allow for the targeted delivery of a drug moiety to a tissues (such as a tumor), and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemo therapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour: 14 (3): 154-169; Chari, R. V. (2008) ACC. Chen. Res. 41.98-107.

In the context of treating cancer, the ADC compounds of the application include those with anticancer activity. In some embodiments, the ADC compounds include an antibody conjugated, i.e. covalently attached, to the drug moiety. In some embodiments, the antibody is covalently attached to the drug moiety through a linker. In some embodiments, the second agent is connected to the anti-SIRPα antibody moiety via a linker (such as a linker described herein). In some embodiments, the linker is a cleavable. In some embodiments, the linker is non-cleavable.

The antibody-drug conjugates (ADC) of the application selectively deliver an effective dose of a drug to tumor tissue whereby greater selectivity, i.e. a lower efficacious dose, may be achieved while increasing the therapeutic index (“therapeutic window’). The drug moiety of the antibody-drug conjugates (ADC) may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, pyrrolobenzodiazepine (PBD), nemorubicin and its derivatives, PNU-159682, anthracycline, duocarmycin, Vinca alkaloid, taxane, trichothecene, CC1065, camptothecin, elinafide, and stereoisomers, isos teres, analogs, and derivatives thereof that have cytotoxic activity.

Production of immunoconjugates described herein can be found in, for example, U.S. Pat. Nos. 9,562,099 and 7,541,034, which are hereby incorporated by references in their entirety.

### Linkers

In some embodiments, the anti-SIRPα constructs described herein comprise one or more linkers between two moieties (e.g., the anti-SIRPα antibody moiety and the half-life extending moiety, the anti-SIRPα antibody moiety and the second binding moiety in the multispecific constructs described above). The length, the degree of flexibility and/or other properties of the linker(s) used in the anti-SIRPα constructs may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### a) Peptide Linkers

The peptide linker may have a naturally occurring sequence, or a non-naturally occurring sequence. For example, a sequence derived from the hinge region of heavy chain only antibodies may be used as the linker. See, for example, WO1996/34103. The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9 (1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 68), glycine-serine polymers (including, for example, (GS)ₙ (SEQ ID NO: 69), (GSGGS)ₙ (SEQ ID NO: 70), (GGGGS)ₙ (SEQ ID NO: 71), and (GGGS)ₙ (SEQ ID NO: 72), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (See Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure.

Furthermore, exemplary linkers also include the amino acid sequence of such as (GGGGS)ₙ (SEQ ID NO: 71), wherein n is an integer between 1 and 8, e.g. (GGGGS)₃ (SEQ ID NO: 73; hereinafter referred to as “(G4S) 3” or “GS3”), or (GGGGS)₆ (SEQ ID NO: 74; hereinafter referred to as “(G4S) 6” or “GS6”). In some embodiments, the peptide linker comprises the amino acid sequence of (GSTSGSGKPGSGEGS)ₙ (SEQ ID NO: 75), wherein n is an integer between 1 and 3.

### b) Nonpeptide Linkers

Coupling of two moieties may be accomplished by any chemical reaction that will bind the two molecules so long as both components retain their respective activities, e.g., binding to SIRPα and a second agent in an anti-SIRPα multispecific antibody, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (See Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC. In some embodiments, the linker is a PEG containing linker.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### 5.4. Methods of Preparation

The present disclosure provides methods of preparing the presently disclosed anti-SIRPα constructs or antibody moieties that specifically binds to SIRPα and compositions such as polynucleotides, nucleic acid constructs, vectors, host cells, or culture media that are produced during the preparation of the anti-SIRPα constructs or antibody moieties. The anti-SIRPα construct or antibody moiety or composition described herein may be prepared by a number of processes as generally described below and more specifically in the Examples.

### 5.4.1. Antibody Expression and Production

The antibodies (including anti-SIRPα monoclonal antibodies, anti-SIRPα bispecific antibodies, and anti-SIRPα antibody moieties) described herein can be prepared using any known methods in the art, including those described below and in the Examples.

### a) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also See Example 1 for immunization in Camels.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as flow cytometry, radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA), enzyme-linked assay (ELISA), or BLI. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, ion exchange chromatography, gel electrophoresis, dialysis, or affinity chromatography.

Monoclonal antibodies may also be made by recombinant DNA methods, such as those described in U.S. Pat. No. 4,816,567, and as described above. mRNA encoding the monoclonal antibodies is readily isolated and sequenced using conventional procedures (e.g., by using oligonucleotide probes that are capable of binding specifically to cDNA encoding the heavy and light chains of murine antibodies). The hybridoma cells serve as a preferred source of such mRNA. Once isolated, the cDNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### 5.4.2. Nucleic Acid Molecules Encoding Antibody Moieties

The present disclosure provides a polynucleotide encoding any one of the anti-SIRPα constructs or antibody moieties described herein. In some embodiments, the present disclosure provides a polynucleotide prepared using any one of the methods as described herein. In some embodiments, a nucleic acid molecule comprises a polynucleotide that encodes a heavy chain or a light chain of an antibody moiety (e.g., anti-SIRPα antibody moiety). In some embodiments, a nucleic acid molecule comprises both a polynucleotide that encodes a heavy chain and a polynucleotide that encodes a light chain, of an antibody moiety (e.g., anti-SIRPα antibody moiety). In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody moiety (e.g., anti-SIRPα antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### 5.4.3. Nucleic Acid Constructs

The present disclosure further provides a nucleic acid construct comprising any one of the polynucleotides described herein. In some embodiments, the present disclosure provides a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### 5.4.4. Vectors

The present disclosure provides a vector comprising any polynucleotides that encode the heavy chains and/or light chains of any one of the antibody moieties described herein (e.g., anti-SIRPα antibody moieties) or nucleic acid construct described herein. The present disclosure provides a vector prepared using any method described herein. Vectors comprising polynucleotides that encode any of anti-SIRPα constructs such as antibodies, scFvs, fusion proteins or other forms of constructs described herein (e.g., anti-SIRPα scFv) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a first polynucleotide that encodes a heavy chain and a second vector comprises a second polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

### 5.4.5. Host Cells

The present disclosure provides a host cell comprising any polypeptide, nucleic acid construct and/or vector described herein. The present disclosure provides a host cell prepared using any method described herein. In some embodiments, the host cell is capable of producing any of antibody moieties described herein under a fermentation condition.

In some embodiments, the antibody moieties described herein (e.g., anti-SIRPα antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, CHOZN® and FUT8 CHO cells; PER.C6® cells (Crucell); and NSO cells. In some embodiments, the antibody moieties described herein (e.g., anti-SIRPα antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

The present disclosure also provides host cells comprising any of the polynucleotides or vectors described herein. In some embodiments, the present disclosure provides a host cell comprising an anti-SIRPα antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

In some embodiments, the antibody moiety is produced in a cell-free system. Non-limiting exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

### 5.4.6. Culture Media

The present disclosure provides a culture medium comprising any antibody moiety, polynucleotide, nucleic acid construct, vector, and/or host cell described herein. The present disclosure provides a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium. In some embodiments, the medium is a chemically defined medium. In some embodiments, the chemically defined medium is optimized for the host cell line.

### 5.4.7. Purification of Antibody Moieties

The anti-SIRPα constructs (e.g., anti-SIRPα monoclonal antibodies or multispecific antibodies) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-SIRPα construct comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### 5.5. Methods of Treatment

The present disclosure in one aspect provides methods of treating a disease or condition (such as cancer or infectious disease) in an individual, comprising administering to the individual an effective amount of an anti-SIRPα construct (such as any of the anti-SIRPα constructs described herein). Please note that the methods described herein, while described generically for conciseness purposes, is intended to independently apply to each of the anti-anti-SIRPα constructs described here.

In some embodiments, there is provided a method of treating a tumor (such as a solid tumor, a cancer, e.g., a colon cancer, melanoma, or a T cell lymphoma) in an individual, comprising administering into the individual an effective amount of an anti-SIRPα construct (such as any of the anti-SIRPα constructs described herein). In some embodiments, the anti-SIRPα construct is a monoclonal antibody. In some embodiments, the anti-SIRPα construct is a fusion protein or immunoconjugate comprising an anti-SIRPα antibody moiety and a second moiety, such as a second moiety comprising a cytokine (such as a pro-inflammatory cytokine). In some embodiment, the SIRPα is a human SIRPα. In some embodiments, the tumor tissue has an increased expression level of SIRPα as compared to a reference tissue (such as a corresponding tissue in a healthy individual). In some embodiments, the tumor is an advanced or malignant tumor. In some embodiments, the tumor is a cancer. In certain embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering to the individual a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the second agent comprises a cell comprising a chimeric antigen receptor that specifically binds to a tumor antigen. In some embodiments, the anti-SIRPα construct and the second agent are administered simultaneously or concurrently. In some embodiments, the anti-SIRPα construct and the second agent are administered sequentially. In some embodiments, the anti-SIRPα construct and/or the second agent are administered parentally. In some embodiments, the anti-SIRPα construct is administered to a diseased tissue directly.

In some embodiments, the present disclosure provides a method of treating an infectious disease (such as a viral infectious disease) in an individual, comprising administering into the individual an effective amount of an anti-SIRPα construct (such as any of the anti-SIRPα constructs described herein). In some embodiments, the anti-SIRPα construct comprises an anti-SIRPα antibody. In some embodiments, the anti-SIRPα antibody is a monoclonal antibody. In some embodiments, the anti-SIRPα construct is a fusion protein or immunoconjugate comprising an anti-SIRPα antibody moiety and a second moiety. In some embodiments, the second moiety comprises a cytokine (such as a pro-inflammatory cytokine). In some embodiment, the SIRPα is a human SIRPα. In some embodiments, the infection site has an increased expression level of SIRPα as compared to a reference tissue (such as a corresponding tissue in a healthy individual). In some embodiments, the method further comprises administering to the individual a second agent. In some embodiments, the second agent comprises an immune therapy. In some embodiments, the anti-SIRPα construct and the second agent are administered simultaneously or concurrently. In some embodiments, the anti-SIRPα construct and the second agent are administered sequentially. In some embodiments, the anti-SIRPα construct and/or the second agent are administered parentally. In some embodiments, the anti-SIRPα construct is administered to a diseased tissue directly.

The administration of the anti-SIRPα constructs described herein can also be useful for promoting local immune response, promoting proliferation and/or activation of immune cells (such as T cells), and promoting a favorable tumor microenvironment. In some embodiments, the present disclosure provides a method of promoting local immune response in a cancer tissue in an individual having a cancer (such as a solid tumor), comprising administering any of the anti-SIRPα constructs described herein. In some embodiments, the present disclosure provides a method of promoting local immune response in an infection site in an individual having an infection (such as a virus infection), comprising administering to an individual having an infection any of the anti-SIRPα constructs described herein.

In some embodiments, the present disclosure provides a method of promoting a favorable tumor microenvironment in a cancer tissue in an individual having a tumor (such as a solid tumor), comprising administering into the individual any of the anti-SIRPα constructs described herein. In some embodiments, the present disclosure provides a method of promoting a favorable microenvironment in an infection site in an individual having an infection (such as a virus infection), comprising administering to the individual any of the anti-SIRPα constructs described herein. “Promoting favorable tumor microenvironment” generally refers to or comprises conversion of a tumor tissue that is resistant to a cancer therapy (such as an immunotherapy) to a tumor tissue that is less resistant to the cancer therapy.

### A. Disease or Condition

The methods described herein are applicable to diseases and conditions for which there are suppressed immune responses in the body that at least partly contribute to the less effective treating of the disease. Exemplary diseases include cancer or infectious disease (such as viral infectious disease).

### 1. Cancer

In some embodiments, the disease or condition described herein is a tumor. In some embodiments, the disease or condition described herein is a cancer. Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the tumor is a solid tumor.

In some embodiments, the tumor is a liquid tumor.

In some embodiments, the tumor tissue has a high expression level of SIRPα when the expression level of SIRPα (e.g., assessed by immunohistochemistry) is at least about 5%, 10%, 15%, 20%, 25%, 30%, 40%, 50%, 60%, 70%, 80%, 90%, or 95% higher than the expression level of SIRPα in a reference tissue. In some embodiments, the tumor tissue has a high expression level of SIRPα when the expression level of SIRPα (e.g., assessed by immunohistochemistry) is at least about 1-fold, 2-fold, 3-fold, 4-fold, 5-fold, 6-fold, 7-fold, 8-fold, 9-fold, 10-fold, 20-fold, 30-fold, 40-fold, or 50-fold higher than the expression level of SIRPα in a reference tissue. In some embodiments, the reference tissue is the corresponding tissue in a healthy individual. In some embodiments, the expression level of SIRPα in a reference tissue is the average expression level of SIRPα in the same tissue in a group of individuals (such as 10, 30, 50, 100 individuals) with same or similar cancer. In some embodiments, the reference tissue is the corresponding tissue in an individual who also has a tumor but has a less suppressed immune response in the tumor tissue as indicated by a biomarker (such as high M2 macrophages, or high expression of an immune checkpoint agent such as PD-1 or PD-L1).

In some embodiments, the tumor tissue has a high T cell infiltration (e.g., high CD3 T cells, high CD8 T cells, high CD4 T cells, activated T cells, activated CD8 T cells, or activated CD4 T cells). In some embodiments, the tumor tissue has a high T cell infiltration when the number of the T cells in the tumor is at least about 5%, 10%, 15%, 20%, 25%, 30%, 40%, 50%, 60%, 70%, 80%, 90%, or 95% more than the number of the corresponding T cells in a reference tissue. In some embodiments, the high T cell infiltration is present when the number of the T cells in the cancer is at least about 1-fold, 2-fold, 3-fold, 4-fold, 5-fold, 6-fold, 7-fold, 8-fold, 9-fold, or 10-fold more than the number of the corresponding T cells in a reference tissue. In some embodiments, the reference tissue is the corresponding tissue in a healthy individual. In some embodiments, the number of the corresponding T cells in a reference tissue is the average number of the corresponding T cells in the same tissue in a group of individuals (such as 10, 30, 50, 100 individuals) with same or similar tumor. In some embodiments, the reference tissue is the corresponding tissue in an individual who also has a tumor but has a less suppressed immune response in the tumor tissue as indicated by a biomarker (such as high M2 macrophages, high expression of an immune checkpoint agent such as PD-1 or PD-L1, high expression level of SIRPα).

In some embodiments, the tumor has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the tumor tissue as compared to that of a reference tissue. In some embodiments, the tumor has a decreased number (such as a decrease by at least 5%, 10%, 15%, 20%, 25%, 30%, 40%, 50%, 60%, 70%, 80%, 90%, or 95%) of activated immune cells (such as activated T cells, activated CD4+ T cells, or activated CD8+ T cells) in the tumor tissue as compared to that of a reference tissue. In some embodiments, the tumor tissue has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) as compared to that of a reference tissue.

In some embodiments, the reference tissue is the corresponding tissue in a healthy individual. In some embodiments, the reference tissue is the corresponding tissue in an individual who also has a tumor but has a less suppressed immune response in the tumor tissue. The suppression of immune response can be assessed by measuring a) the number of immune cells (e.g., CD3+ cells); b) the proliferating/expanding status of immune cells; c) the activation status of immune cells; and/or d) the cytokine level. In some embodiments, any one or more of the a)-d) is measured in the tumor tissue. In some embodiments, the immune cells are T cells. In some embodiments, the immune cells are CD8+ T cells (such as activated CD8+ T cells). In some embodiments, the immune cells are CD4+ T cells (such as activated CD4+ T cells).

Examples of cancers that may be treated by the methods of this application include, but are not limited to, anal cancer, astrocytoma (e.g., cerebellar and cerebral), basal cell carcinoma, bladder cancer, bone cancer, (osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer, cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the disease or condition is a colon cancer.

In some embodiments, the disease or condition is melanoma.

In some embodiments, the disease or condition is a T cell lymphoma.

### 2. Infectious Disease

In some embodiments, the disease or condition is an infectious disease. In some embodiments, the infectious disease is a viral infectious disease.

In some embodiments, the viral infectious disease is characterized by infection with hepatitis virus, human immunodeficiency virus (HIV), picornavirus, poliovirus, enterovirus, human Coxsackie virus, influenza virus, rhinovirus, echovirus, rubella virus, encephalitis virus, rabies virus, herpes virus, papillomavirus, polyoma virus, RSV, adenovirus, yellow fever virus, dengue virus, parainfluenza virus, hemorrhagic virus, pox virus, varicella zoster virus, parainfluenza virus, reovirus, orbivirus, rotavirus, parvovirus, African swine fever virus, measles, coronavirus (such as SARS-COV, MERS-COV, 2019-nCOV), Ebola virus, mumps or Norwalk virus. In some embodiments, the viral infectious disease is characterized by infection with an oncogenic virus such as CMV, EBV, HBV, KSHV, HPV, MCV, HTLV-1, HIV-1, or HCV. In some embodiments, the one or more genes encoding proteins involved in the viral infectious disease development and/or progression include, but are not limited to, genes encoding RSV nucleocapsid, Pre-gen/Pre-C, Pre-S1, Pre-S2/S,X, HBV conserved sequences, HIV Gag polyprotein (p55), HIV Pol polyprotein, HIV Gag-Pol precursor (p160), HIV matrix protein (MA, p17), HIV capsid protein (CA, p24), HIV spacer peptide 1 (SP1, p2), HIV nucleocapsid protein (NC, p9), HIV spacer peptide 2 (SP2, p1), HIV P6 protein, HIV reverse transcriptase (RT, p50), HIV RNase H (p15), HIV integrase (IN, p31), HIV protease (PR, p10), HIV Env (gp160), gp120, gp41, HIV transactivator (Tat), HIV regulator of expression of virion proteins (Rev), HIV lentivirus protein R (Vpr), HIV Vif, HIV negative factor (Nef), HIV virus protein U (Vpu), human CCR5, miR-122, EBOV polymerase L, VP24, VP40, GP/sGP, VP30, VP35, NPC1, and TIM⁻¹, including mutants thereof.

In some embodiments, the viral infectious disease is characterized by infection with coronavirus. In some embodiments, the viral infectious disease is characterized by infection with influenza virus.

An infection site refers to a tissue in the body where virus appear in a significant number and/or causes significant damages. In some embodiments, the infection site comprises has an increased expression level of SIRPα as compared to a reference tissue. In some embodiments, the SIRPα expression level in the infection site is increased by at least about 10%, 20%, 30%, 40%, 50%, 60%, 70%, 80%, 90% as compared to that of the reference tissue. In some embodiments, the SIRPα expression level in the infection site is increased by at least about 1-fold, 2-fold, 3-fold, 4-fold, or 5-fold as compared to that of the reference tissue.

In some embodiments, the infection site has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the infection site as compared to that of a reference tissue. In some embodiments, the infection site has a decreased number (such as a decrease by at least 5%, 10%, 15%, 20%, 25%, 30%, 40%, 50%, 60%, 70%, 80%, 90%, or 95%) of activated immune cells (such as activated T cells, activated CD4+ T cells, or activated CD8+ T cells) in the infection site as compared to that of a reference tissue. In some embodiments, the infection site has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) as compared to that of a reference tissue.

In some embodiments, the reference tissue is the corresponding tissue in a healthy individual. In some embodiments, the reference tissue is the corresponding tissue in an individual who also has a virus infection (such as a virus infection of the same type) but has a less suppressed immune response in the infection site. The suppression of immune response can be assessed by measuring a) the number of immune cells; b) the proliferating/expanding status of immune cells; c) the activation status of immune cells; and/or d) the cytokine level. In some embodiments, the immune cells in circulation are assessed. In some embodiments, the immune cells in diseased tissue are assessed. In some embodiments, the immune cells in lymph tissue (such as lymph node) are assessed. In some embodiments, the immune cells are T cells. In some embodiments, the immune cells are CD8+ T cells (such as activated CD8+ T cells). In some embodiments, the immune cells are CD4+ T cells (such as activated CD4+ T cells).

### B. Individual

In some embodiments, the individual is a mammal (such as a human).

In some embodiments, the individual is selected for treatment based upon a high expression of SIRPα in a diseased tissue. In some embodiments, the tissue is a cancer tissue. In some embodiments, the tissue is an infection site.

In some embodiments, the SIRPα expression level in the infection site is increased by at least about 10%, 20%, 30%, 40%, 50%, 60%, 70%, 80%, 90% as compared to that of the reference tissue. In some embodiments, the SIRPα expression level in the infection site is increased by at least about 1-fold, 2-fold, 3-fold, 4-fold, or 5-fold as compared to that of the reference tissue.

In some embodiments, the individual is selected for treatment based upon the indication of a suppressed immune response. In some embodiments, the individual has a suppressed immune response in a diseased tissue. In some embodiments, the tissue is a cancer tissue. In some embodiments, the tissue is an infection site.

As described above, the suppression of immune response can be assessed by measuring a) the number of immune cells; b) the proliferating/expanding status of immune cells; c) the activation status of immune cells; and/or d) the cytokine level. In some embodiments, the immune cells in circulation are assessed. In some embodiments, the immune cells in diseased tissue are assessed. In some embodiments, the immune cells in lymph tissue (such as lymph node) are assessed. In some embodiments, the immune cells are T cells. In some embodiments, the immune cells are CD8+ T cells (such as activated CD8+ T cells). In some embodiments, the immune cells are CD4+ T cells (such as activated CD4+ T cells).

In some embodiments, the individual has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the tissue (such as the cancer tissue or infection site) as compared to that of a reference tissue. In some embodiments, the individual has a decreased number (such as a decrease by at least 5%, 10%, 15%, 20%, 25%, 30%, 40%, 50%, 60%, 70%, 80%, 90%, or 95%) of activated immune cells (such as activated T cells, activated CD4+ T cells, or activated CD8+ T cells) in the tissue (such as the cancer tissue or infection site) as compared to that of a reference tissue. In some embodiments, the individual has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) in the tissue (such as the cancer tissue or infection site) as compared to that of a reference tissue.

In some embodiments, the reference tissue is the corresponding tissue in a healthy individual. In some embodiments, the reference tissue is the corresponding tissue in an individual who also has a same or similar disease or condition but has a less suppressed immune response in the disease or condition tissue.

In some embodiments, the individual has a compromised immune system.

In some embodiments, the individual is at least about 60, 65, 70, 75, 80, 85, or 90 years old.

In some embodiments, the individual has at least one prior therapy. In some embodiments, the prior therapy comprises a radiation therapy, a chemotherapy and/or an immunotherapy. In some embodiments, the individual is resistant, refractory, or recurrent to the prior therapy.

### C. Combination Therapy

The present disclosure also provides methods administering an effective amount of an anti-SIRPα construct into an individual for treating a disease or condition (such as tumor (e.g., cancer) or infectious disease), wherein the method further comprises administering to the individual a second agent or therapy. In some embodiments, the second agent or therapy is a standard or commonly used agent or therapy for treating the disease or condition.

In some embodiments, the anti-SIRPα construct is administered simultaneously with the second agent or therapy. In some embodiments, the anti-SIRPα construct is administered concurrently with the second agent or therapy. In some embodiments, the anti-SIRPα construct is administered sequentially with the second agent or therapy.

### D. Exemplary Combination Therapies for Tumors

In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a cell therapy (such as a cell therapy comprising an immune cell (e.g., CAR T cell)). In some embodiments, the second agent or therapy comprises an angiogenesis inhibitor.

In some embodiments, the second agent is selected from the group consisting of a chemotherapeutic agent, an immunomodulator, an anti-angiogenesis agent, a growth inhibitory agent, and an antineoplastic agent.

In some embodiments, the second agent is a chemotherapeutic agent. In some embodiments, the second agent is an antimetabolite agent. In some embodiments, the antimetabolite agent is 5-FU.

In some embodiments, the second agent is an immunomodulator. In some embodiments, the immunomodulatory is an immune checkpoint inhibitor.

In some embodiments, the second agent comprises a cell (such as an immune cell, such as a T cell) comprising a chimeric antigen receptor that specifically binds to a tumor antigen.

### E. Exemplary Combination Therapies for Infectious Diseases (Such as Viral Infectious Diseases).

In some embodiments, the second agent or therapy comprises a nucleotide analogue. In some embodiments, the second agent or therapy comprises a nucleoside analogue.

In some embodiments, the second agent or therapy comprises a protease inhibitor. In some embodiments, the second agent or therapy comprises Lopinavir. In some embodiments, the second agent or therapy comprises ritonavir.

In some embodiments, the second agent or therapy comprises a neuraminidase inhibitor. In some embodiments, the second agent or therapy comprises zanamivir. In some embodiments, the second agent or therapy comprises oseltamivir. In some embodiments, the second agent or therapy comprises peramivir.

In some embodiments, the second agent or therapy comprises a Cap-dependent endonuclease inhibitor. In some embodiments, the second agent or therapy comprises baloxavir.

In some embodiments, the second agent or therapy comprises a sialidase.

The second agent and the anti-SIRPα construct can be administered sequentially, concurrently, or simultaneously. In some embodiments, the second agent is administered prior to the anti-SIRPα construct. In some embodiments, the second agent is administered after the anti-SIRPα construct.

### F. Dosing Regimen and Routes of Administration

The dose of the anti-SIRPα construct and, in some embodiments, the second agent as described herein, administered to an individual (such as a human) may vary with the particular composition, the method of administration, and the particular kind and stage of disease or condition being treated. The amount should be sufficient to produce a desirable response, such as a therapeutic response against the disease or condition. In some embodiments, the amount of the anti-SIRPα construct and/or the second agent is a therapeutically effective amount.

In some embodiments, the amount of the anti-SIRPα construct is an amount sufficient to decrease the suppression of the immune response in the individual. Whether there is a decrease in the suppression of the immune response and the extent of the decrease in suppression can be indicated by any of the following.

In some embodiments, the amount of the anti-SIRPα construct is an amount sufficient to increase the number of immune cells (such as T cells, such as CD4+ and/or CD8+ T cells) by at least about 10%, 20%, 30%, 40%, 50%, 60%, 70%, 80%, 90%, or 100% post administration of the anti-SIRPα construct. In some embodiments, the immune cells in circulation are assessed. In some embodiments, the immune cells in a diseased tissue are assessed. In some embodiments, the immune cells in a lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the number of immune cells is assessed about 1, 2, 3, 4, 5, 6, or 7 days post administration of the anti-SIRPα construct.

In some embodiments, the amount of the anti-SIRPα construct is an amount sufficient to increase the number of activated immune cells (such as activated CD4+ and/or CD8+ T cells) by at least about 10%, 20%, 30%, 40%, 50%, 60%, 70%, 80%, 90%, or 100% post administration of the anti-SIRPα construct. In some embodiments, the activated immune cells in circulation are assessed. In some embodiments, the activated immune cells in diseased tissue are assessed. In some embodiments, the activated immune cells in lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the number of activated immune cells is assessed about 1, 2, 3, 4, 5, 6, or 7 days post administration of the anti-SIRPα construct.

In some embodiments, the amount of the anti-SIRPα construct is an amount sufficient to increase the proliferation of immune cells or activated immune cells by at least about 10%, 20%, 30%, 40%, 50%, 60%, 70%, 80%, 90%, or 100% post administration of the anti-SIRPα construct. In some embodiments, the immune cells or the activated immune cells in circulation are assessed. In some embodiments, the immune cells or the activated immune cells in diseased tissue are assessed. In some embodiments, the immune cells or the activated immune cells in lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the proliferation of immune cells or activated immune cells is assessed about 1, 2, 3, 4, 5, 6, or 7 days post administration of the anti-SIRPα construct.

In some embodiments, the amount of the anti-SIRPα construct is an amount sufficient to increase the cytokine level (such as a pro-inflammatory cytokine, such as IFNγ or IL-2) by at least about 10%, 20%, 30%, 40%, 50%, 60%, 70%, 80%, 90%, or 100% post administration of the anti-SIRPα construct. In some embodiments, the cytokine level in diseased tissue is assessed. In some embodiments, the level of cytokine is assessed about 1, 2, 3, 4, 5, 6, or 7 days post administration of the anti-SIRPα construct.

In some embodiments, the amount of the anti-SIRPα construct is an amount sufficient to decrease the suppressive immune cells by at least about 10%, 20%, 30%, 40%, 50%, 60%, 70%, 80%, or 90% post administration. In some embodiments, the suppressive immune cells comprise regulatory T cells. In some embodiments, the suppressive immune cells comprise myeloid derived suppressor cells. In some embodiments, the suppressive immune cells in circulation are assessed. In some embodiments, the suppressive immune cells in diseased tissue are assessed. In some embodiments, the suppressive immune cells in lymph tissue (such as lymph node) are assessed. In some embodiments, the number of suppressive immune cells is assessed about 1, 2, 3, 4, 5, 6, or 7 days post administration of the anti-SIRPα construct.

In some embodiments, the amount of the anti-SIRPα construct is an amount sufficient to increase humoral immune response in the individual by at least about 10%, 20%, 30%, 40%, 50%, 60%, 70%, 80%, 90%, or 100% post administration of the anti-SIRPα construct. The humoral immune response can be assessed by measuring antibodies (such as IgG antibodies) that target a disease-associated antigen or plasmablasts that produce such antibodies in circulation. In some embodiments, the humoral immune response is assessed about 7-28 days (such as about 7-14 days) post administration of the anti-SIRPα construct.

In some embodiments, the amount of the anti-SIRPα construct is an amount sufficient to produce a decrease of the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same individual prior to treatment or compared to the corresponding activity in other individuals not receiving the treatment.

In some embodiments, the anti-SIRPα construct is administered at a dose of about 0.001 μg/kg to about 100 mg/kg of total body weight, for example, about 0.005 μg/kg to about 50 mg/kg, about 0.01 μg/kg to about 10 mg/kg, or about 0.01 μg/kg to about 1 mg/kg.

In some embodiments according to any one of the methods described herein, the anti-SIRPα construct and/or the second agent composition is administered intravenously, intraarterially, intraperitoneally, intravesicularly, subcutaneously, intrathecally, intrapulmonarily, intramuscularly, intratracheally, intraocularly, topically, transdermally, orally, or by inhalation. In some embodiments, the anti-SIRPα construct and/or the second agent is administered intravenously.

In some embodiments, the anti-SIRPα construct is administered directly to the diseased tissue.

### 5.6. Compositions, Kits and Articles of Manufacture

Also provided herein are compositions (such as formulations) comprising any one of the anti-SIRPα construct or anti-SIRPα antibody moiety described herein, nucleic acid encoding the antibody moieties, vector comprising the nucleic acid encoding the antibody moieties, or host cells comprising the nucleic acid or vector.

Suitable formulations of the anti-SIRPα construct described herein can be obtained by mixing the anti-SIRPα construct or anti-SIRPα antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes. Also provided are kits comprising any one of the anti-SIRPα construct or anti-SIRPα antibody moiety described herein. The kits may be useful for any of the methods of modulating cell composition or treatment described herein.

In some embodiments, the present disclosure provides a kit comprising an anti-SIRPα construct specifically binding to SIRPα.

In some embodiments, the kit further comprises a device capable of delivering the anti-SIRPα construct into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., cancer, infectious disease, autoimmune disease, or transplantation.

The kits of the present disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present disclosure thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

The examples below are intended to be purely exemplary of the application and should therefore not be considered to limit the application in any way. The following examples and detailed description are offered by way of illustration and not by way of limitation.

### Example 1. Generation of Anti-SIRPα Antibodies

### Hybridoma Generation

To generate antibodies against SIRPα, Balb/c mice, NZB/Wmice, Lewis Rats or humanized transgenic mice were immunized with either recombinant human or mouse SIRPα Fc-tagged proteins. Animals were immunized at multiple sites using Freund's complete adjuvant (CFA) or RIBI adjuvant for multiple boosts. Test bleeds were done by saphenous vein lancing seven days after the last boost. When antibody titer was high enough, mice were given a final IV boost via lateral tail vein. Four days after the final boost, immunized animals were sacrificed and spleens isolated. Hybridomas were generated by electrofusion of splenocytes with Sp2/0 myeloma cells. Fused cells were plated into 96-well plates in HAT selective medium and grown for 10⁻¹⁴ days to generate hybridoma clones.

### Enzyme-linked Immunosorbent Assays (ELISA)

Hybridoma clones were assayed for binding to human SIRPα proteins by ELISA. Human SIRPα V1, SIRPα V2, SIRPα V8, SIRPβ, SIRPγ or cynomolgus monkey SIRPα (Sino Biological) were coated on 96-Well ELISA plates (Corning) at 0.5 μg/mL in PBS and incubated overnight at 4° C. Wells were washed twice in PBS containing 0.5% Tween-20 (PBST) and blocked with PBS containing 5% BSA for one hour at room temperature. After washing, supernatant was added and incubated for 1 hour at room temperature. Supernatants were then removed and wells washed three times with PBST, followed by addition of horseradish peroxidase-conjugated goat anti-mouse IgG antibodies or goat anti-rat IgG (Jackson Immuno) at an optimized dilution in PBS containing 0.5% BSA for one hour. Wells were washed three times with PBST, developed with 50 μl ABTS substrate (Sigma Aldrich) for 15-30 minutes at room temperature and read at 405 nm. Parental hybridomas reactive to SIRPα proteins were subcloned by limiting dilution and their binding reconfirmed by ELISA. Positive binders were expanded and antibodies in the supernatants were purified by protein G (HiTrap Protein G HP, GE Healthcare).

Flow cytometry

Supernatants from ELISA-positive clones or purified antibodies were further tested for their ability to bind to human SIRPα polymorphic variants (V1, V2 and V8) or human SIRPγ expressed on the cell surface of 293 cells (293-hSIRPα) or Jurkat cells, respectively, by flow cytometry. The cells were harvested and 1×10⁵ cells per well plated in 96-well U-bottom plates and the supernatant removed. 50 μl of hybridoma supernatant or purified antibody at the specified dilution or concentration was added to each well and incubated at 4° C. for 30 minutes. Assay plates were washed 2 times with PBS and antibody binding was detected using a FITC-labeled goat anti-mouse antibody or goat anti-rat antibody (Biolegend) at 1:400 dilution in PBS for 30 minutes at 4° C. Plates were washed 2 times and analyzed on a BD LSRFortessa X-20 flow cytometer (Becton Dickinson).

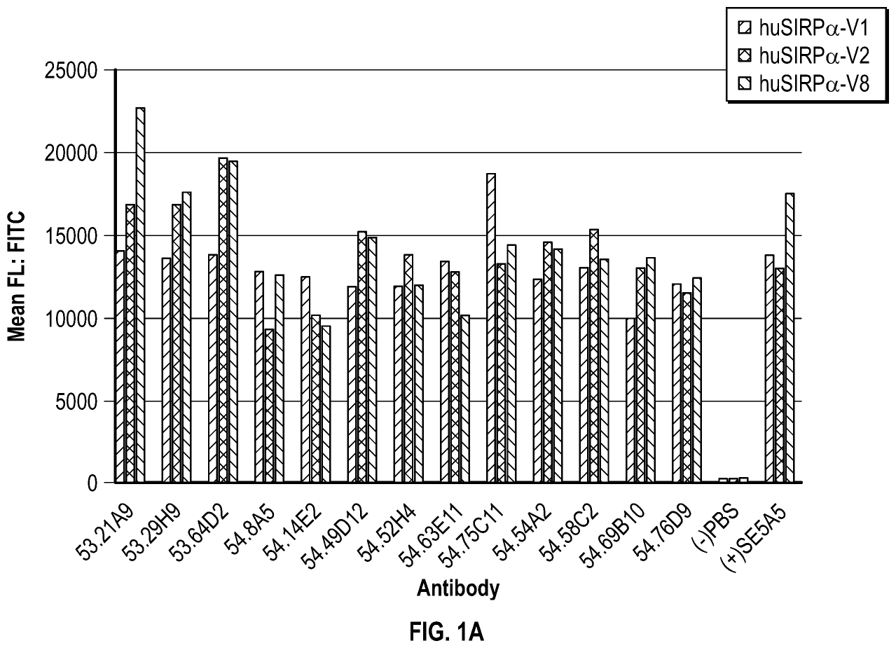

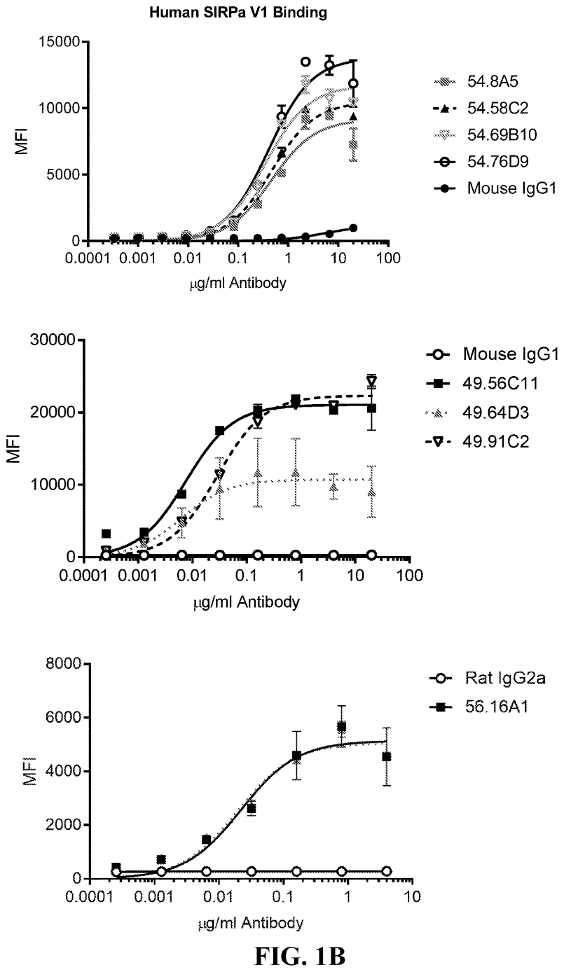

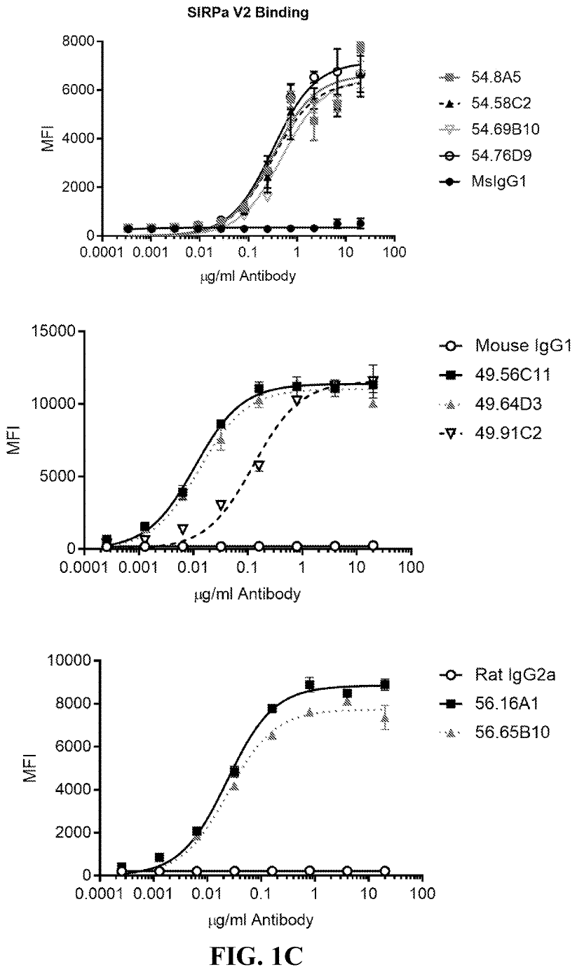

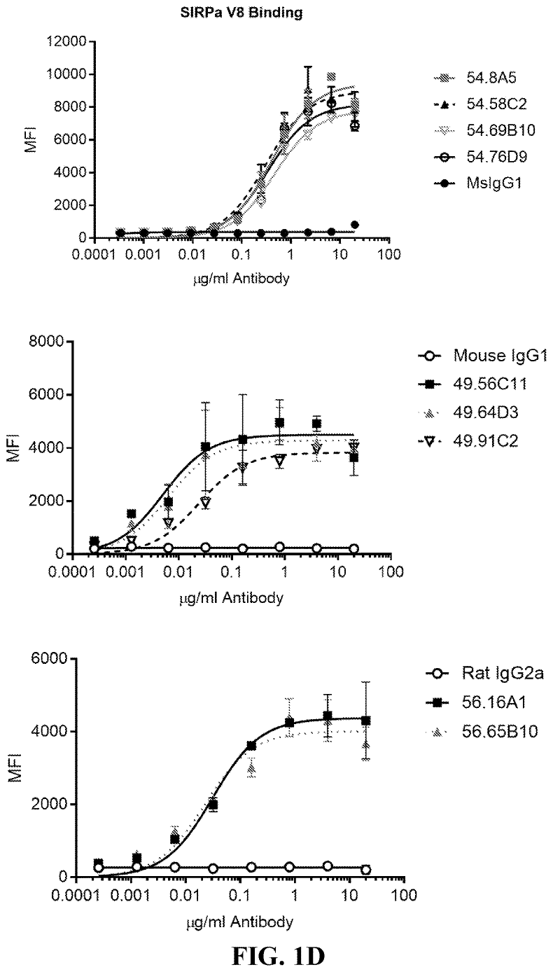

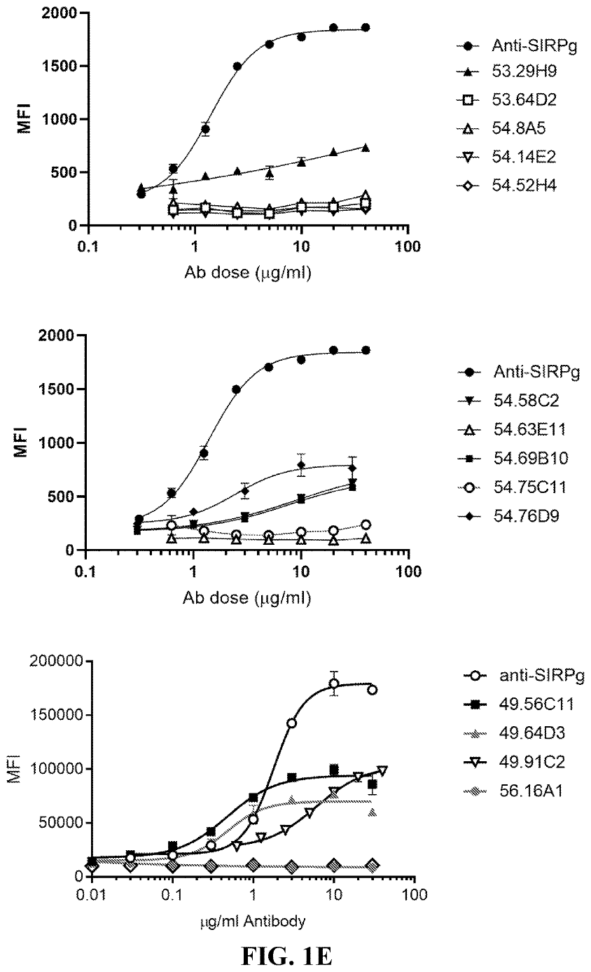

All antibodies harboring sequences shown in Tables 3-5 were capable of binding to all three variants of human SIRPα. As shown in FIGS. 1A-1D, representative antibodies all bound to V1, V2, and V8 polymorphic variants of 293-hSIRPα cells. None of the antibodies bind to mouse SIRPα (data not shown). Also, as shown in FIG. 1E, none of the antibodies tested bound to SIRPγ-expressing Jurkat cells strongly, as compared to anti-SIRPγ antibody LSB2.20. Sequence Analysis

Sequence data from all constructs were analyzed and consensus sequences for heavy and light chain were determined. See Tables 3-5 that list V_{H}, V_{L}, and CDRs sequences of the various anti-SIRPα antibodies and consensus sequences.

TABLE 3
V_{H} CDRs of various antibodies and consensus sequences

| | CDRH1 | CDRH2 | CDRH3 |
|---|---|---|---|
| Group A | | | |
| 53.21A9.B11 | SGRYYWS | YIYYSGSTNYNPSLKS | AYDWNYFFDY |
| | (SEQ ID NO: 1) | (SEQ ID NO: 2) | (SEQ ID NO: 3) |
| Group B | | | |
| 53.29H9.C7 | GYYWS | EINHSGSTNYNPSLKR | GRGYSGYYYFDY |
| | (SEQ ID NO: 9) | (SEQ ID NO: 10) | (SEQ ID NO: 11) |
| Group C | | | |
| 53.64D2.A7 | SYDMD | GIGIAGDTYYPDSVKG | GGGWDGSLDY |
| | (SEQ ID NO: 17) | (SEQ ID NO: 18) | (SEQ ID NO: 19) |
| 54.8A5.A5 | SYDMH | AIGTAGDTYYPGSVKG | GGNWDDAFDI |
| | (SEQ ID NO: 25) | (SEQ ID NO: 26) | (SEQ ID NO: 27) |
| 54.14E2.A11 | SYDMH | VIGIAGDTYYTGSVKG | GGNWDDAFDI |
| | (SEQ ID NO: 25) | (SEQ ID NO: 31) | (SEQ ID NO: 27) |
| 54.49D12.A6 | SYDMH | VIGTAGDTYFPGSVKG | GGNWDDAFDI |
| | (SEQ ID NO: 25) | (SEQ ID NO: 37) | (SEQ ID NO: 27) |
| 54.52H4.B4 | SYDMH | AIGTAGDTYYPGSVKG | GGNWDDAFDI |
| | (SEQ ID NO: 25) | (SEQ ID NO: 26) | (SEQ ID NO: 27) |
| 54.54A2.F9 | SYDMH | AIGTAGDTYYPGSVKG | GGNWDDAFDI |
| | (SEQ ID NO: 25) | (SEQ ID NO: 26) | (SEQ ID NO: 27) |
| 54.58C2.B9.A2 | SYDMH | VIGISGDTYYPGSVKG | GGNWDDAFDI |
| | (SEQ ID NO: 25) | (SEQ ID NO: 47) | (SEQ ID NO: 27) |
| 54.63E11.D9 | SYDMH | VIGISGDTYYPGSVKG | GGNWDDAFDI |
| | (SEQ ID NO: 25) | (SEQ ID NO: 47) | (SEQ ID NO: 27) |
| 54.69B10.E7 | SYDMH | VIGIAGDTYYAGSVKG | GGNWDDAFDI |
| | (SEQ ID NO: 25) | (SEQ ID NO: 53) | (SEQ ID NO: 27) |
| 54.75C11.B7 | SYDMH | VIGIAGDTYYAGSVKG | GGNWDDAFDI |
| | (SEQ ID NO: 25) | (SEQ ID NO: 53) | (SEQ ID NO: 27) |
| 54.76D9.B6 | SYDMH | VIGISGDTYYPGSVKG | GGNWDDALDI |
| | (SEQ ID NO: 25) | (SEQ ID NO: 47) | (SEQ ID NO: 59) |
| Group C | SYDMX₁ | X₁IGX₂X₃GDTYX₄X₅GSVKG | GGNWDDAX₁DI |
| consensus | X₁ = D or H | X₁ = A or V | X₁ = D or L |
| sequence | (SEQ ID NO: 62) | X₂ = I or T | (SEQ ID NO: 64) |
| | | X₃ = A or S | Or SEQ ID NO: 19 |
| | | X₄ = Y or F | |
| | | X₅ = A, P, or T | |
| | | (SEQ ID NO: 63) | |
| | | Or SEQ ID NO: 18 | |
| Group D | | | |
| 49.56C11.C1 | NHAMS | TFGSGGSYTYYLDSVKG | SGWDGWFAY |
| | (SEQ ID NO: 78) | (SEQ ID NO: 79) | (SEQ ID NO: 80) |
| 49.64D3.A6 | NYAMS | TFSSGGSYTYYQDSVKG | SGWDGWFAY |
| | (SEQ ID NO: 86) | (SEQ ID NO: 87) | (SEQ ID NO: 80) |
| Group D | NX₁AMS | TFX₁SGGSYTYYX₂DSVKG | SGWDGWFAY |
| consensus | X₁ = H or Y | X₁ = G or S | (SEQ ID NO: 80) |
| sequence | (SEQ ID NO: | X₂ = L or Q | |
| | 108) | (SEQ ID NO: 109) | |
| Group E | | | |
| 49.91C2.A9 | RYWMS | EINPDSSTINYTPSLKD | SFYGSSYWYFDV |
| | (SEQ ID NO: 92) | (SEQ ID NO: 93) | (SEQ ID NO: 94) |
| Group F | | | |
| 56.16A1.H11 | NYDMA | SISYGGSRIYYRDSVKG | DYGYNPSYYWYF |
| | (SEQ ID NO: | (SEQ ID NO: 101) | DF |
| | 100) | | (SEQ ID NO: 102) |

TABLE 4
V_{L} CDRs of various antibodies and consensus sequences.

| | CDRL1 | CDRL2 | CDRL3 |
|---|---|---|---|
| Group A | | | |
| 53.21A9.B11 | RSSQSLLYSNGYNYLE | LGSNRAS | MQGLQTPIT |
| | (SEQ ID NO: 4) | (SEQ ID NO: 5) | (SEQ ID NO: 6) |
| Group B | | | |
| 53.29H9.C7 | RASQSVSSSYLA | GASSRAT | QQYDSSPLT |
| | (SEQ ID NO: 12) | (SEQ ID NO: 13) | (SEQ ID NO: 14) |
| Group C | | | |
| 53.64D2.A7 | RASQDINNYLA | TASRLQS | QQYNTYPYT |
| | (SEQ ID NO: 20) | (SEQ ID NO: 21) | (SEQ ID NO: 22) |
| 54.8A5.A5 | RASQDINNYLA | TASRLQS | QQYTFYPYT |
| | (SEQ ID NO: 20) | (SEQ ID NO: 21) | (SEQ ID NO: 28) |
| 54.14E2.A11 | RASQGINNYLA | TASSLQS | QQYNSYPYT |
| | (SEQ ID NO: 32) | (SEQ ID NO: 33) | (SEQ ID NO: 34) |
| 54.49D12.A6 | RASQGINNYLA | TVSRLQS | QQYNSYPYT |
| | (SEQ ID NO: 32) | (SEQ ID NO: 38) | (SEQ ID NO: 34) |
| 54.52H4.B4 | RASQGINNYLA | TTSSLQS | QQYISYPYT |
| | (SEQ ID NO: 32) | (SEQ ID NO: 41) | (SEQ ID NO: 42) |
| 54.54A2.F9 | RASQGINNYLA | TASSLQS | QQYNSYPYT |
| | (SEQ ID NO: 32) | (SEQ ID NO: 33) | (SEQ ID NO: 34) |
| 54.58C2.B9.A2 | RASQDINNYLA | TTSSLQS | QQYVSYPYT |
| | (SEQ ID NO: 20) | (SEQ ID NO: 41) | (SEQ ID NO: 48) |
| 54.63E11.D9 | RASQDINNYLA | TASSLQS | QQYNTYPYT |
| | (SEQ ID NO: 20) | (SEQ ID NO: 33) | (SEQ ID NO: 22) |
| 54.69B10.E7 | RASQDINNYLA | TTSSLQS | QQYVSYPYT |
| | (SEQ ID NO: 20) | (SEQ ID NO: 41) | (SEQ ID NO: 48) |
| 54.75C11.B7 | RASQDINNYLA | TASSLQS | QQYSTYPYT |
| | (SEQ ID NO: 20) | (SEQ ID NO: 33) | (SEQ ID NO: 56) |
| 54.76D9.B6 | RASQDINNYLA | TTSSLQS | QQYVSYPYT |
| | (SEQ ID NO: 20) | (SEQ ID NO: 41) | (SEQ ID NO: 48) |
| Group C | RASQX₁INNYLA | TX₁SX₂LQS | QQYX₁ X₂YPYT |
| consensus | X₁ = D or G | X₁ = A, V or T, | X₁ = I, N, T, V or S |
| sequence | (SEQ ID NO: 65) | X₂ = R or S | X₂ = F, T or S |
| | | (SEQ ID NO: 66) | (SEQ ID NO: 67) |
| Group D | | | |
| 49.56C11.C1 | KSSQSLLNSNNQKNYL | FASTRES | QQHYSTLPWT |
| | A | (SEQ ID NO: 82) | (SEQ ID NO: 83) |
| | (SEQ ID NO: 81) | | |
| 49.64D3.A6 | KSSQSLLNSSNQKNYL | FASTRES | QQHCSTLPWT |
| | A | (SEQ ID NO: 82) | (SEQ ID NO: 89) |
| | (SEQ ID NO: 88) | | |
| Group D | KSSQSLLNSX₁NQKNY | FASTRES | QQHX₁STLPWT |
| consensus | LA | (SEQ ID NO: 82) | X₁ = Y or C |
| sequence | X₁ = N or S | | (SEQ ID NO: 111) |
| | (SEQ ID NO: 110) | | |
| Group E | | | |
| 49.91C2.A9 | RASESVDNYGISFMN | AASNQGS | QQSKEVPYT |
| | (SEQ ID NO: 95) | (SEQ ID NO: 96) | (SEQ ID NO: 97) |
| Group F | | | |
| 56.16A1.H11 | RASQGISNYLN | YTSNLQS | QQYDSSPYT |
| | (SEQ ID NO: 103) | (SEQ ID NO: 104) | (SEQ ID NO: 105) |

TABLE 5
V_{H} and V_{L} amino acid sequences of various antibodies

| | | V_{H} | V_{L} |
|---|---|---|---|
| 1. | 53.21A9.B11 | SEQ ID NO: 7 | SEQ ID NO: 8 |
| 2. | 53.29H9.C7 | SEQ ID NO: 15 | SEQ ID NO: 16 |
| 3. | 53.64D2.A7 | SEQ ID NO: 23 | SEQ ID NO: 24 |
| 4. | 54.8A5.A5 | SEQ ID NO: 29 | SEQ ID NO: 30 |
| 5. | 54.14E2.A11 | SEQ ID NO: 35 | SEQ ID NO: 36 |
| 6. | 54.49D12.A6 | SEQ ID NO: 39 | SEQ ID NO: 40 |
| 7. | 54.52H4.B4 | SEQ ID NO: 43 | SEQ ID NO: 44 |
| 8. | 54.54A2.F9 | SEQ ID NO: 45 | SEQ ID NO: 46 |
| 9. | 54.58C2.B9.A2 | SEQ ID NO: 49 | SEQ ID NO: 50 |
| 10. | 54.63E11.D9 | SEQ ID NO: 51 | SEQ ID NO: 52 |
| 11. | 54.69B10.E7 | SEQ ID NO: 54 | SEQ ID NO: 55 |
| 12. | 54.75C11.B7 | SEQ ID NO: 57 | SEQ ID NO: 58 |
| 13. | 54.76D9.B6 | SEQ ID NO: 60 | SEQ ID NO: 61 |
| 14. | 49.56C11.C1 | SEQ ID NO: 84 | SEQ ID NO: 85 |
| 15. | 49.64D3.A6 | SEQ ID NO: 90 | SEQ ID NO: 91 |
| 16. | 49.91C2.A9 | SEQ ID NO: 98 | SEQ ID NO: 99 |
| 17. | 56.16A1.H11 | SEQ ID NO: 106 | SEQ ID NO: 107 |

### Example 2. Affinity Determination for Binding of Anti-SIRPα Antibodies to Recombinant Human SIRPα Proteins and Recombinant Cyno SIRPα Protein

### Biacore Measurement

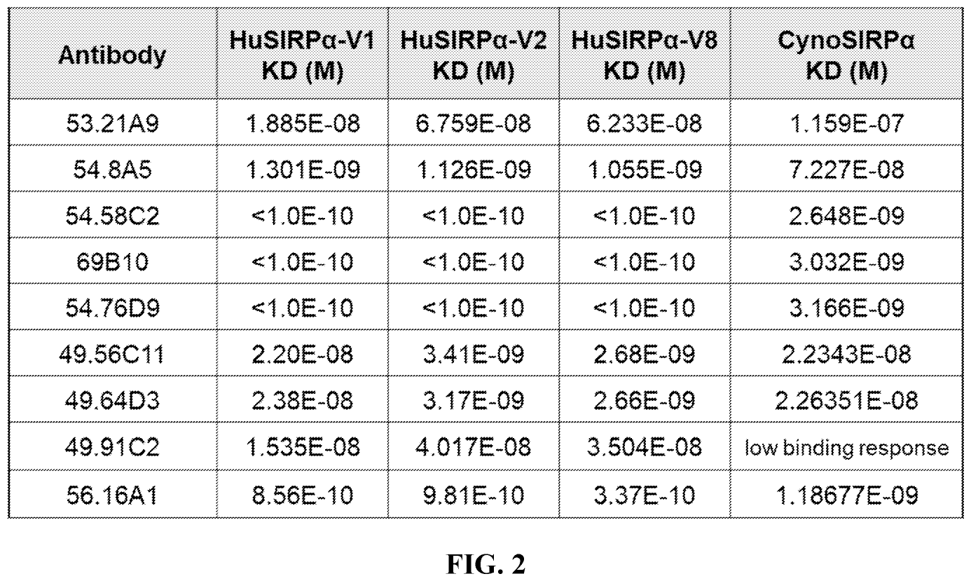

The affinity between antibodies and all three variants of recombinant human SIRPα-His (V1, V2, V8) and cyno SIRP-alpha (SinoBiological) was determined by kinetic analysis on the Biacore™ X100 system. Biosensor analysis was conducted at 25° C. in HBS-EP buffer system (10 mM HEPES pH 7.3, 150 mM NaCl, 3 mM EDTA, 0.05% Surfactant P20). Goat anti-mouse IgG capture antibody (Mouse Antibody Capture Kit, GE Healthcare) or anti-rat antibody was immobilized (8000+/−200 RU) to both flow cells 1 and 2 of the sensor chip using standard amine coupling chemistry. Mouse isotype control in running buffer was captured on flow cell 1 as a reference surface and purified anti-SIRPα antibodies in running buffer was captured on flow cell 2. Ligands both captured at a low density for 30s at a flow rate of 10 μL/min. To collect kinetic binding data, human or cyno SIRPα-His in running buffer was injected over the two flow cells at concentrations starting from 300 nM diluted down in 2-3 fold dilutions and 0 nM at a flow rate of 30 μL/min. The complex was allowed to associate and dissociate for 120 s and 300 s, respectively. One duplicate sample and a buffer blank were flowed over the two surfaces. The surfaces were regenerated with a 180 s injection of regeneration solution. Data were collected at a rate of 1 Hz. The data was fit to a simple 1:1 interaction model using the global data analysis option available within BiaEvaluation 3.1 software. As shown in FIG. 2, the selected anti-SIRPα antibodies demonstrated strong binding and affinity to recombinant human SIRPα protein (all three variants) and recombinant cyno SIRPα protein by Biacore.

### Example 3. Blocking Human CD47 Binding on SIRPα-Expressing Cells

Competition of Anti-SIRPα Antibodies with CD47 on 293-hSIRPα Cells

293 cells overexpressing either human SIRPα V1, V2 or V8 were collected and distributed at 10⁴ cells/well and incubated with anti-human SIRPα antibodies for 30 min at 4° C. Excess of antibody was washed with PBS, and then the cells were incubated with APC-labeled CD47-Fc at 1 μg/ml for 30 min at 4° C. Cells were washed with and resuspended in PBS and analysed by FACS using BD LSRFortessa. To determine if anti-SIRPα antibodies could block CD47 binding to SIRPγ, recombinant SIRPγ (Sino Biological) was coated on 96-Well ELISA plates (Corning) at 2 μg/mL in PBS and incubated overnight at 4° C. Wells were washed twice in PBS containing 0.5% Tween-20 (PBST) and blocked with PBS containing 5% BSA for one hour at room temperature. After washing, anti-SIRPα antibodies were added at the indicated concentrations with fixed concentration of CD47-Fc and incubated for 1 hour at room temperature. Wells were then washed three times with PBST, followed by addition of horseradish peroxidase-conjugated goat anti-human IgG antibodies (Jackson Immuno) at an optimized dilution in PBS containing 0.5% BSA for one hour. Wells were washed three times with PBST, developed with 50 μl ABTS substrate (Sigma Aldrich) for 15-30 minutes at room temperature and read at 405 nm.

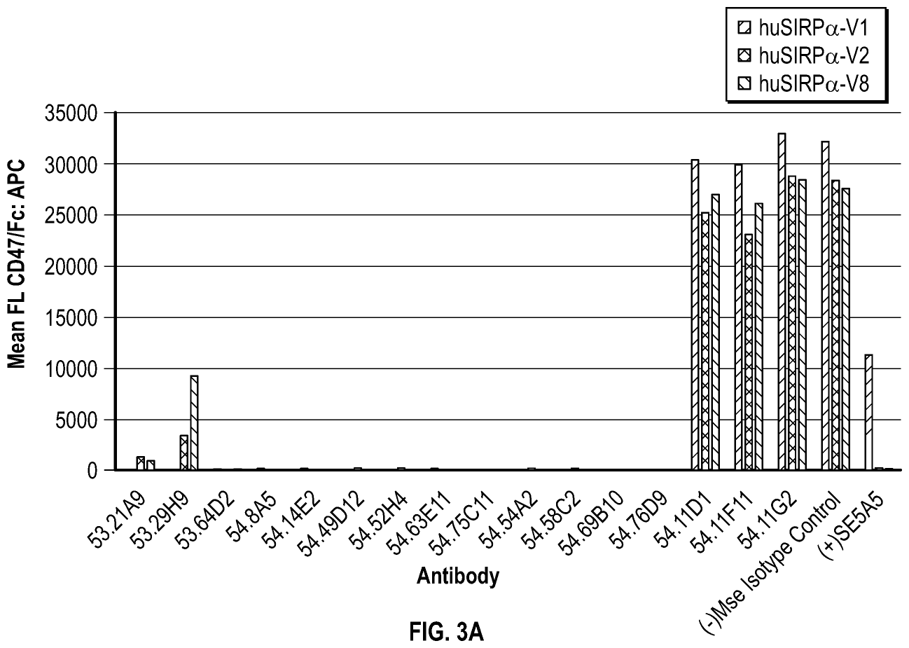

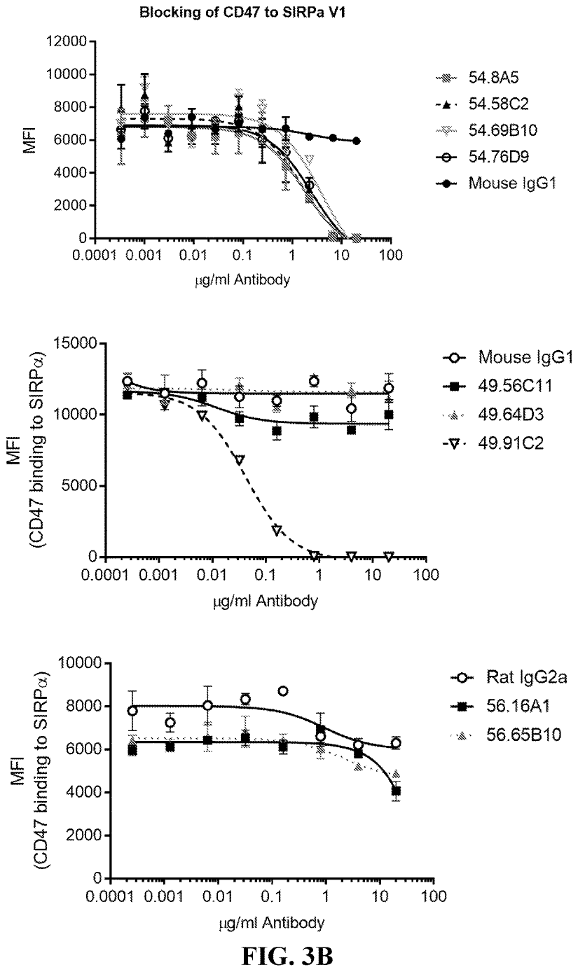

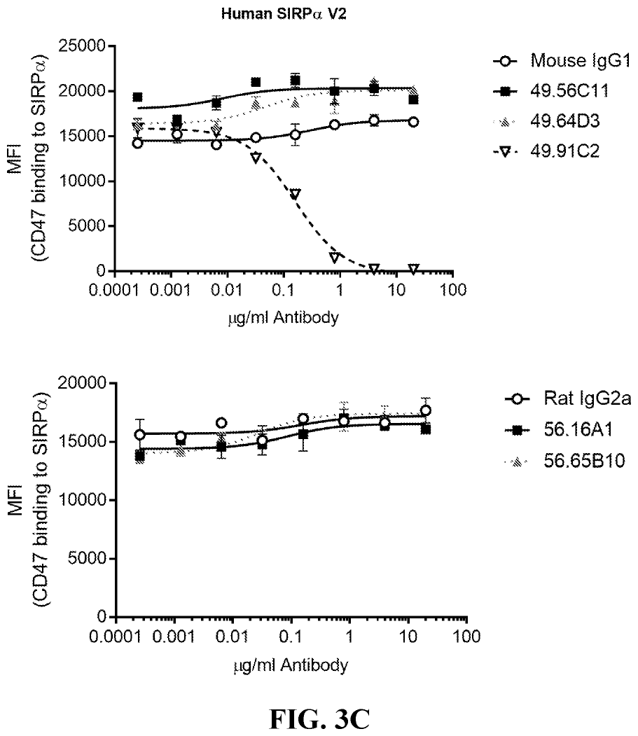

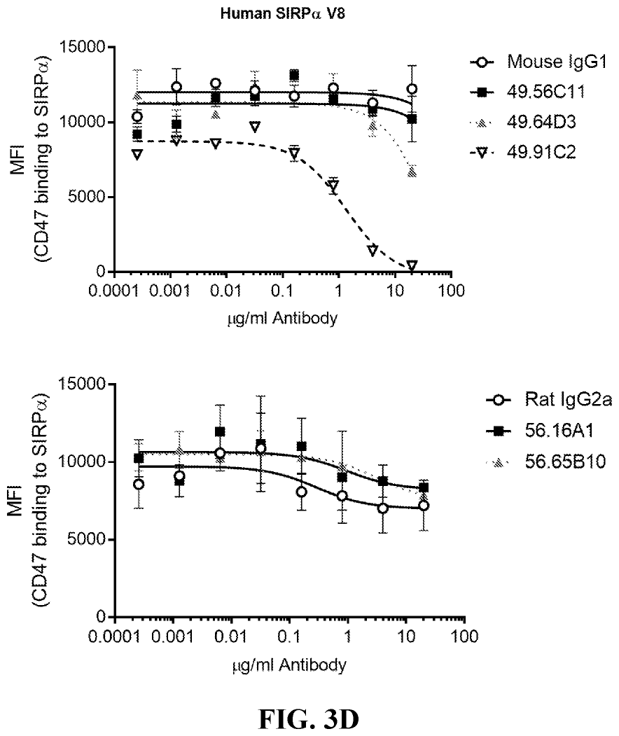

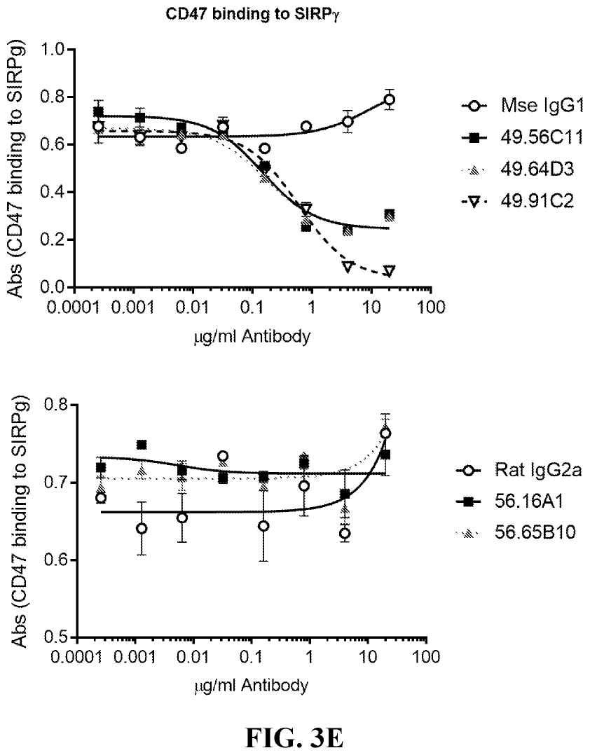

As shown in FIG. 3A, a strong competition by selected anti-SIRPα antibodies with CD47 for binding to all three polymorphic variants (V1, V2 and V8) of membrane expressed SIRPα compared to no antibody added (PBS) and anti-SIRPα antibody, SE5A5. As shown in FIGS. 3B-3D, selected anti-SIRPα antibodies dose-dependently blocked binding of CD47 to SIRPα V1, V2, or V8. Also, FIG. 3E shows that selected anti-SIRPα antibodies do not compete with CD47 for binding to SIRPγ strongly.

### Example 4. In Vitro Functional Characterization of Monoclonal Antibodies

### Phagocytosis Assay

The anti-SIRPα antibodies were tested for their ability to enhance in vitro macrophage phagocytotic activity of tumor cells using cell-based functional assays. The assay in which human macrophages were co-cultured with fluorophore-labeled human tumor cells in the presence of varying concentrations of anti-SIRPα antibodies with or without anti-tumor antibodies were developed. When macrophage phagocytosis occurs, macrophages incorporate tumor cell-derived fluorophore, which were detected by the flow cytometry. The addition of anti-SIRPα antibodies that interfere the CD47-SIRPα interaction should enhance the phagocytotic activities in a dose-dependent manner.

Human monocytes were isolated from the blood of healthy donor volunteers and differentiated to macrophages with M-CSF (BioLegend). EGFR (+) DLD-1 or CD20 (+) Raji cells were labeled with CellTrace CFSE (Invitrogen) according to the manufacturer's instructions, 10⁵ cells were plated into each well of a round bottom 96-well plate, and varying concentration of anti-SIRPα or isotype control antibodies with or without anti-EGFR antibody cetuximab for DLD-1 cells or anti-CD20 antibody rituximab for Raji cells, respectively, were added. Then, 5×10⁴ macrophages were added, and the co-cultures were incubated at 37° C. and 5% CO₂ for 2 hours. The cells were washed twice with PBS to remove excess antibody then incubated with PE-labeled anti-CD206 (BioLegend) and APC-labeled anti-CD33 (BioLegend) antibodies for 30 min at 4° C. The cells were washed again with PBS, resuspended, and analyzed on a BD LSRFortessa X-20 flow cytometer. The SIRPα genotype of macrophages was determined by genome sequencing.

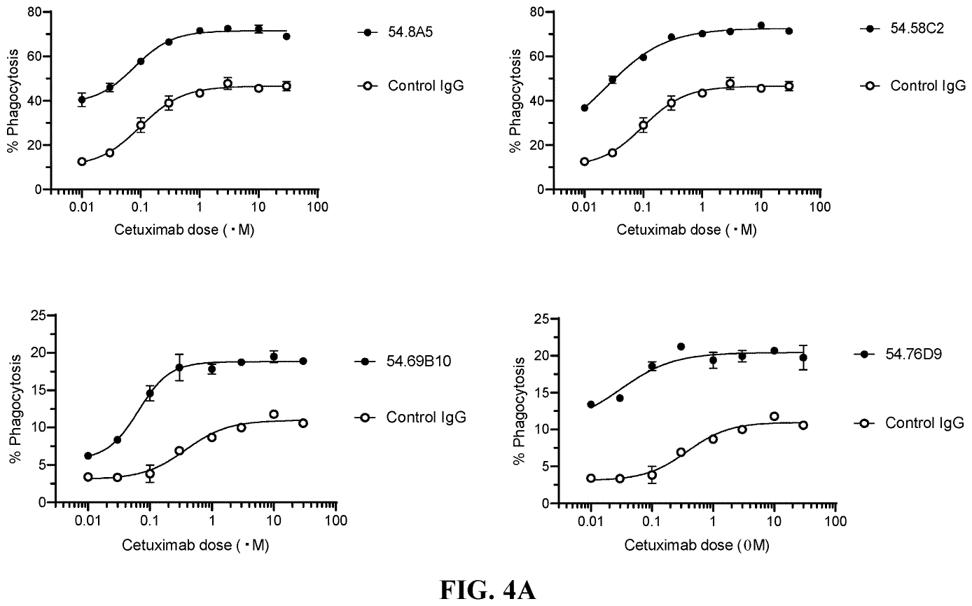

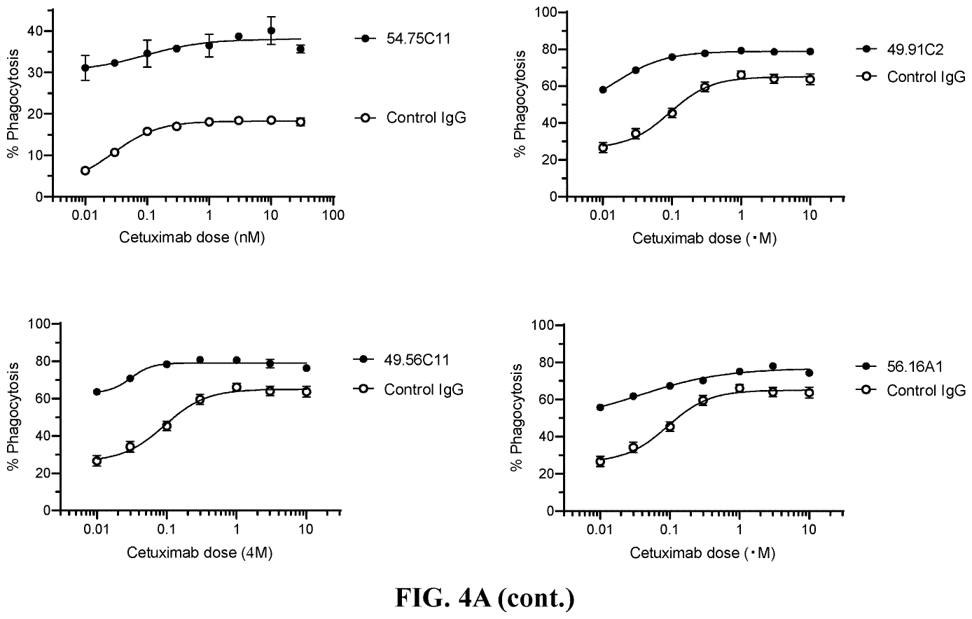

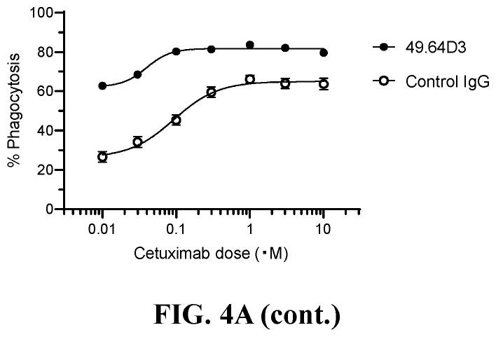

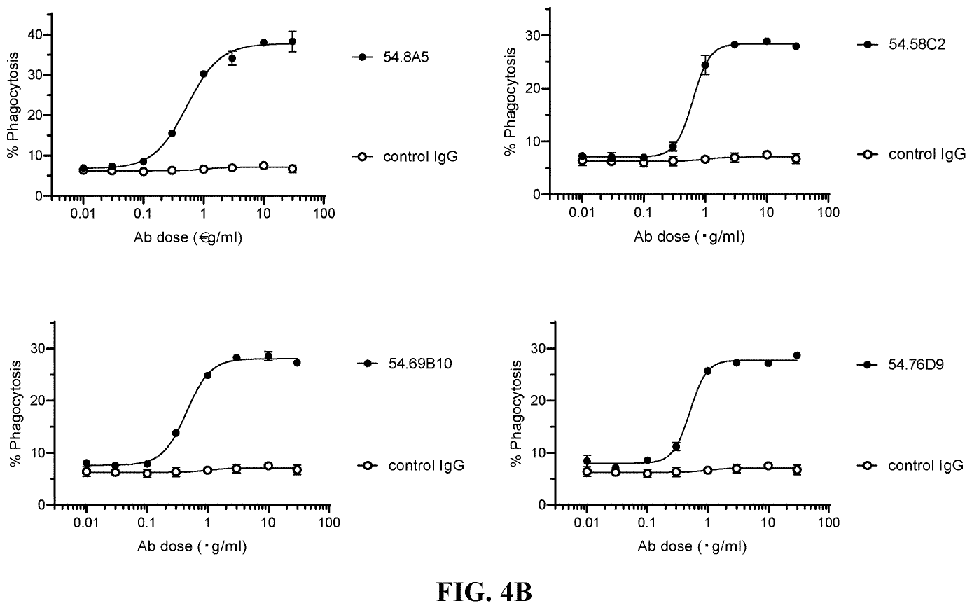

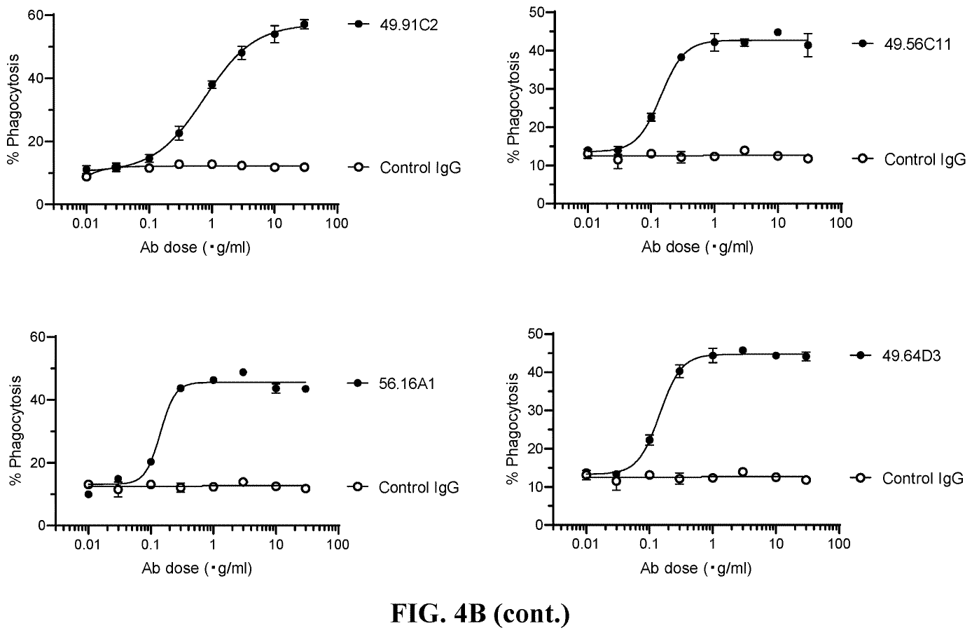

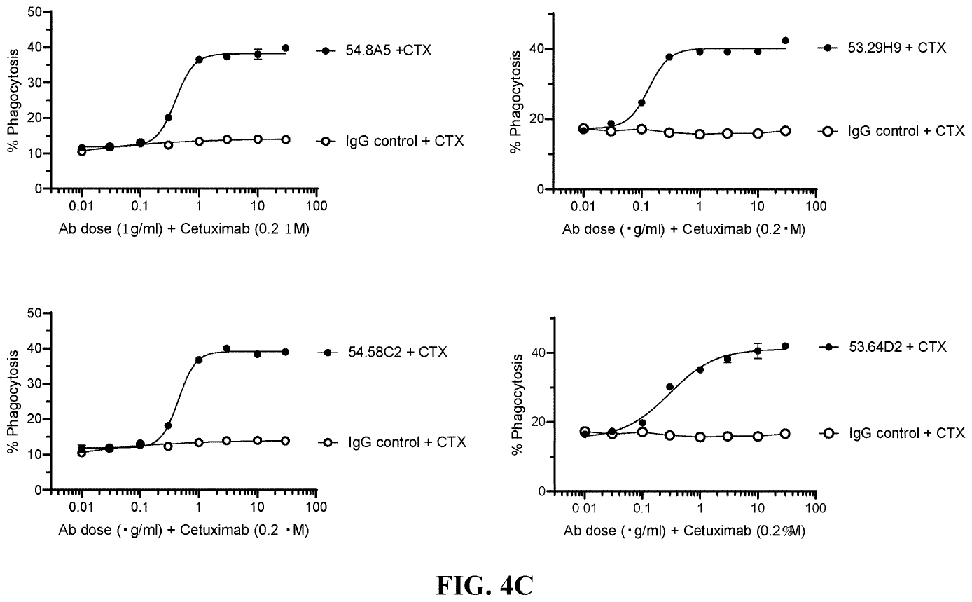

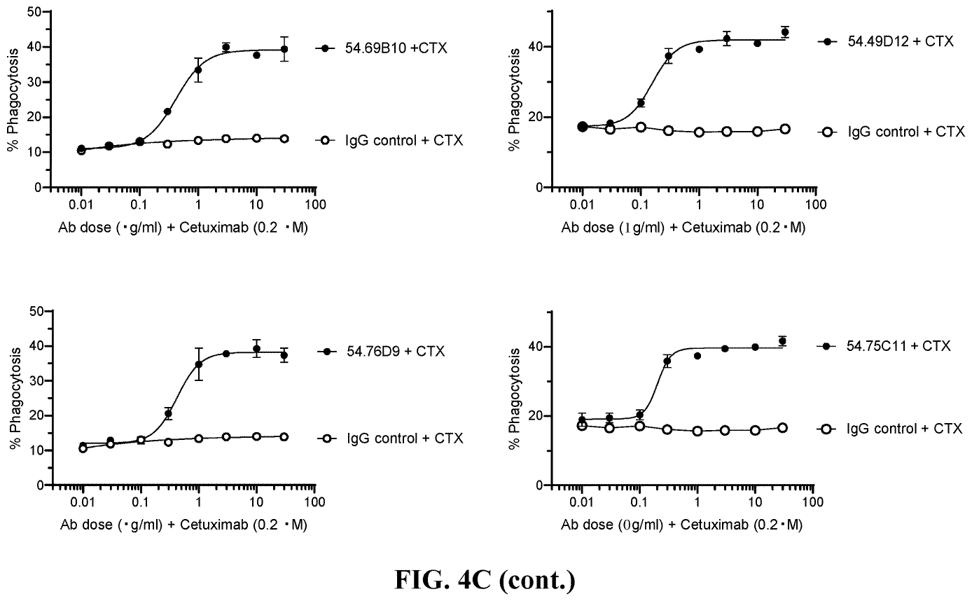

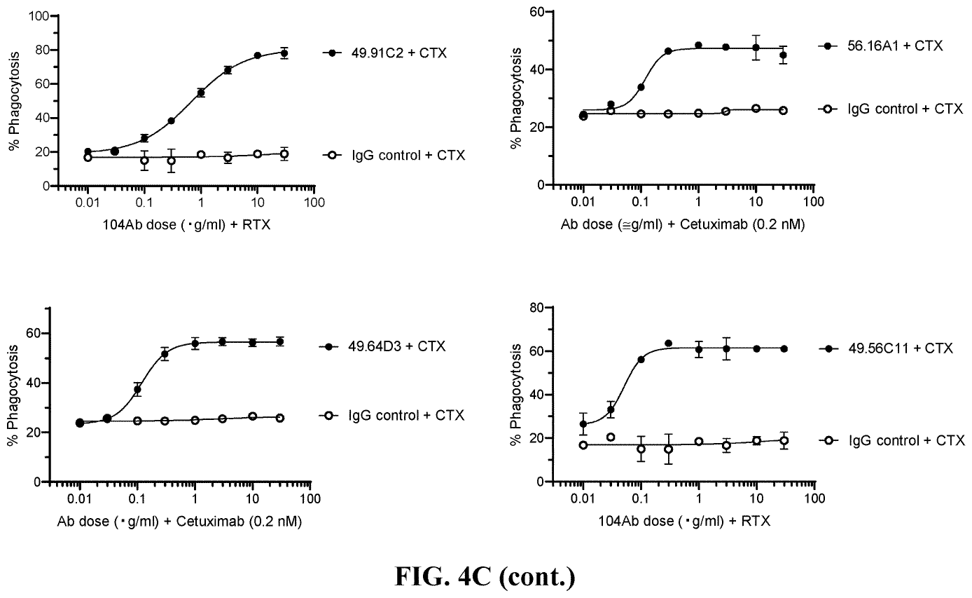

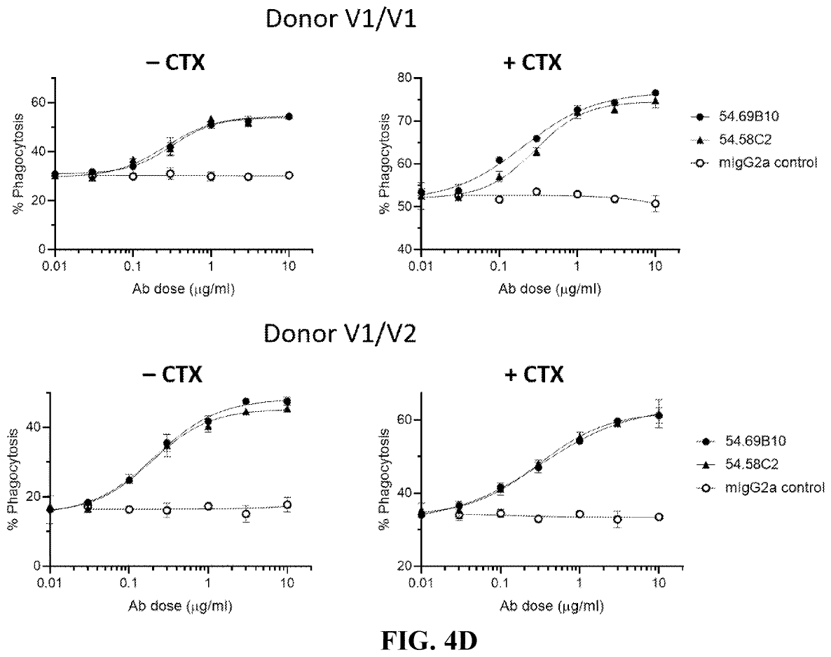

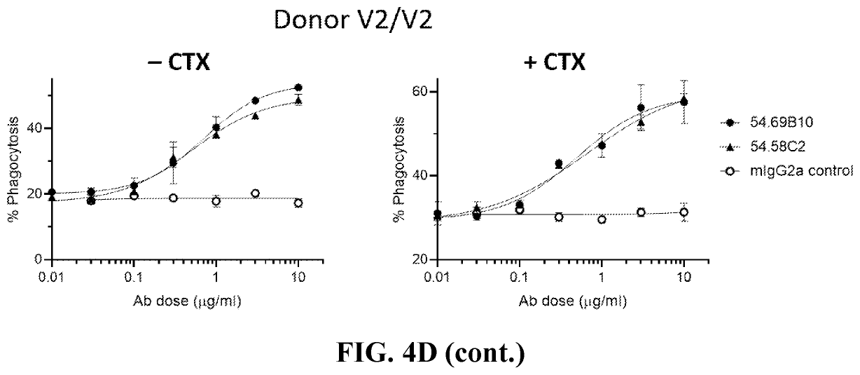

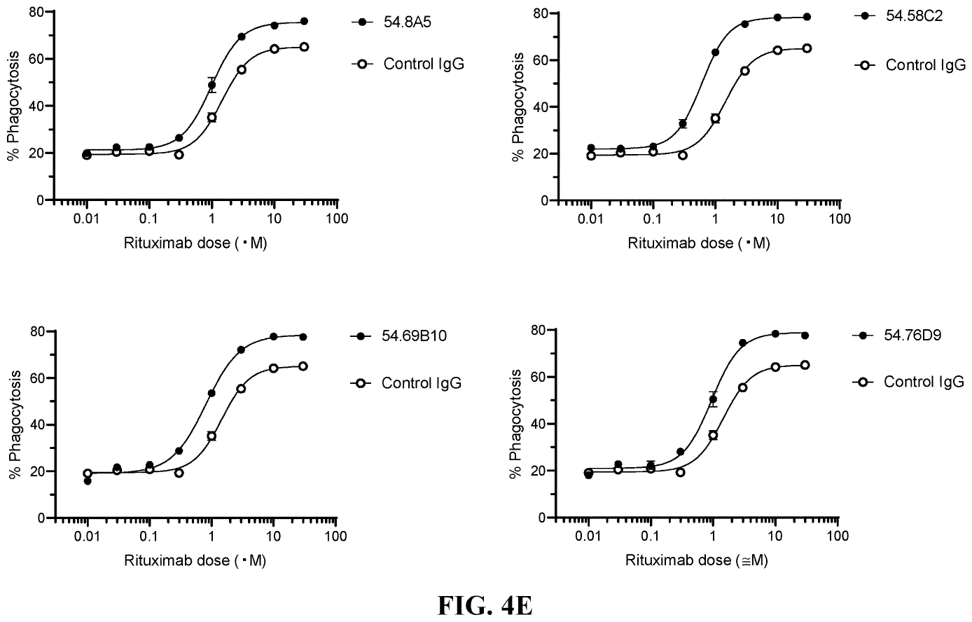

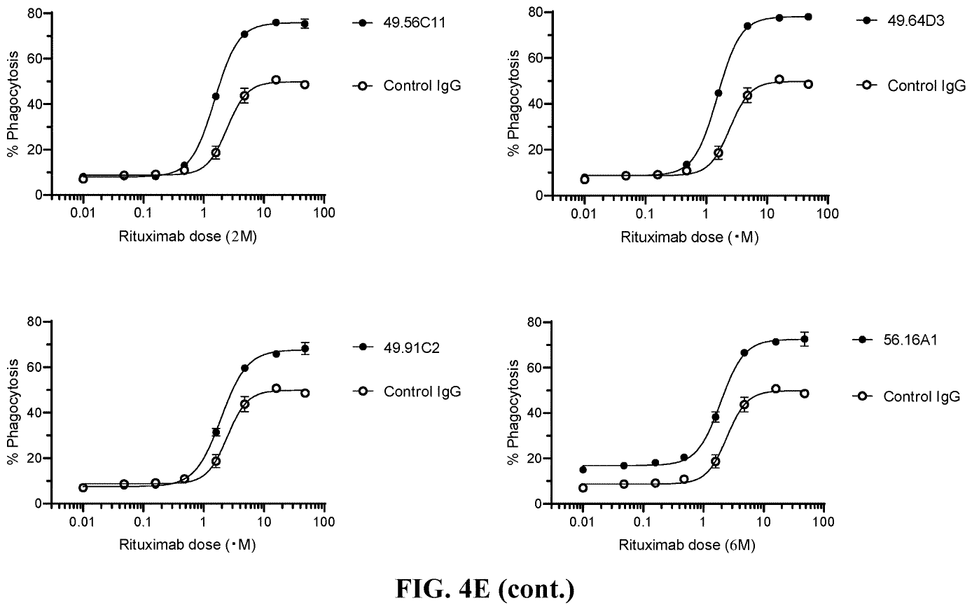

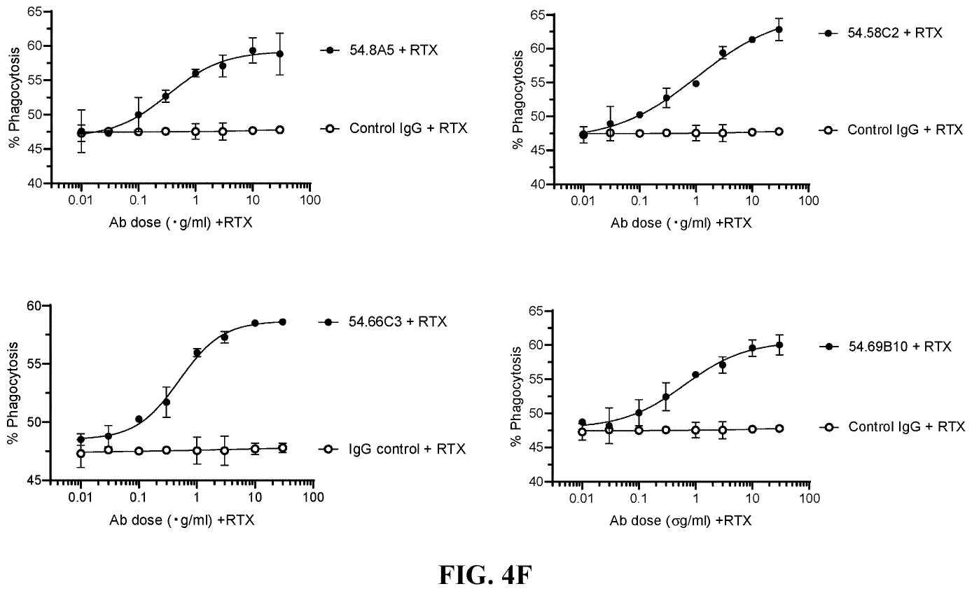

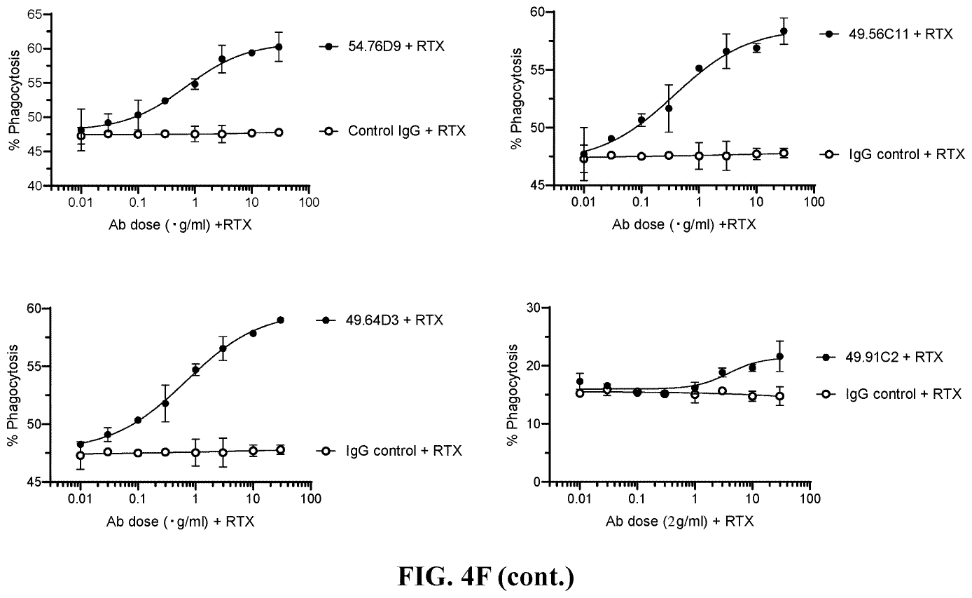

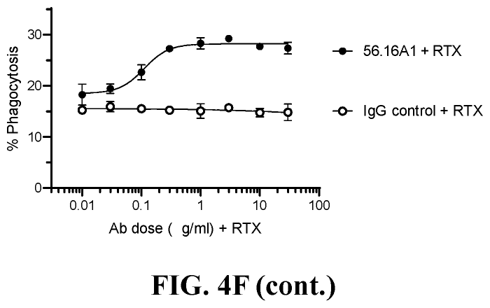

As shown in FIGS. 4A-4F, selected anti-SIRPα antibodies potentiated macrophage phagocytosis of human tumor cells, e.g., human DLD-1 tumor cells (see FIGS. 4A-4D) and Raji tumor cells (see FIGS. 4E-4F). In addition, FIG. 4D shows that selected anti-SIRPα antibodies potentiated phagocytosis of human tumor cells by the macrophages with all available SIRPα polymorphic variants of V1/V1, V1/V2, and V2/V2.

### RBC and Platelet Aggregation Assays

The anti-SIRPα antibodies were characterized for their ability to bind and/or aggregate red blood cells (RBCs) and platelets. RBCs aggregation were detected by imaging the bottom RBC precipitation on the bottom of 96-well plates, and antibody binding to RBCs or platelets were detected by the flow cytometry. The anti-SIRPα antibodies that interfere the CD47-SIRPα interaction but does not bind to CD47 on RBCs and platelets should not affect the RBC and platelet binding and aggregation.

Blood was collected from healthy donor volunteers. RBCs and platelets were obtained by centrifugation for 15 minutes at 500 g and 15 minutes at 2500 g, respectively. For the binding assay, 4×10⁵ RBCs and platelets were plated into each well of a round bottom 96-well plate and incubated with varying concentration of anti-SIRPα or isotype control antibodies for 1 hr at 4° C. Assay plates were washed 2 times with PBS and antibody binding was detected using a PE-labeled anti-mouse antibody (Biolegend) at 1:400 dilution in PBS for 30 minutes at 4° C. Plates were washed 2 times and analyzed on a BD LSRFortessa X-20 flow cytometer. For the aggretination assay, 4×10⁶ RBCs were plated into each well of a round bottom 96-well plate and incubated with anti-SIRPα or anti-CD47 antibodies for 2 hr at 4° C. Then the images were taken using Amersham X Imager.

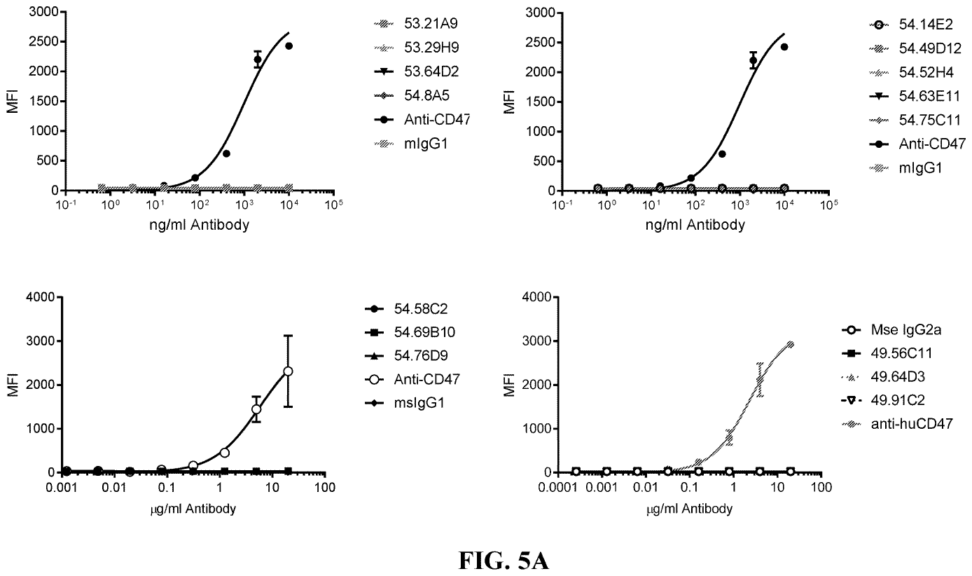

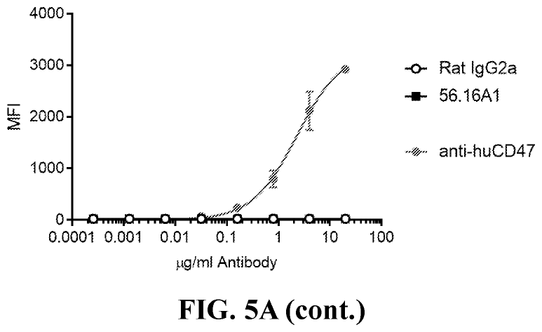

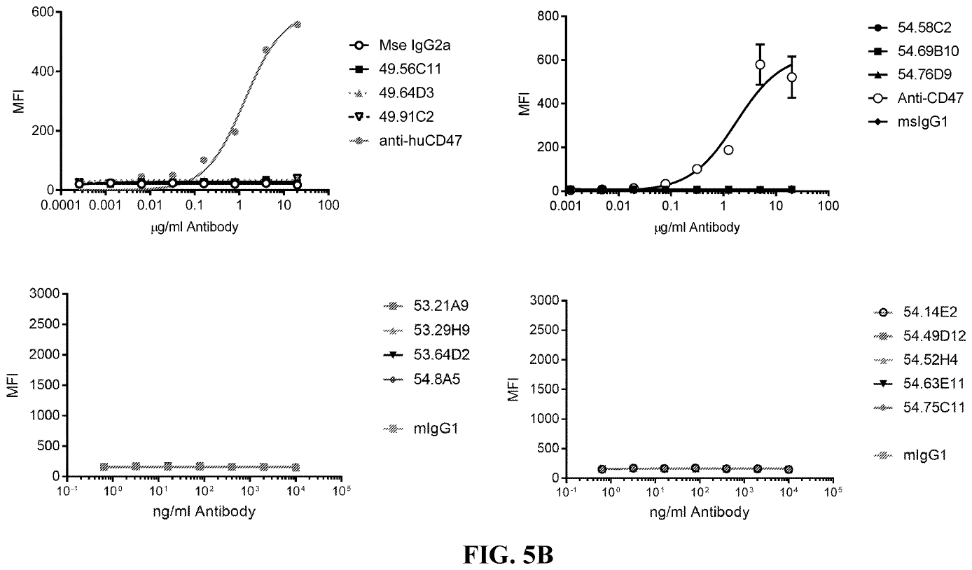

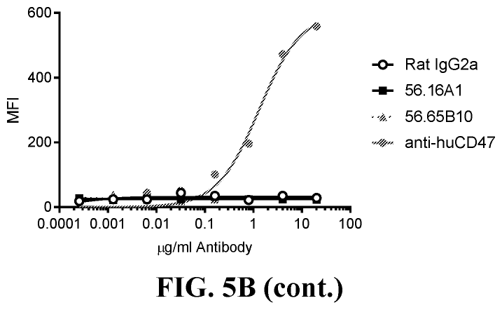

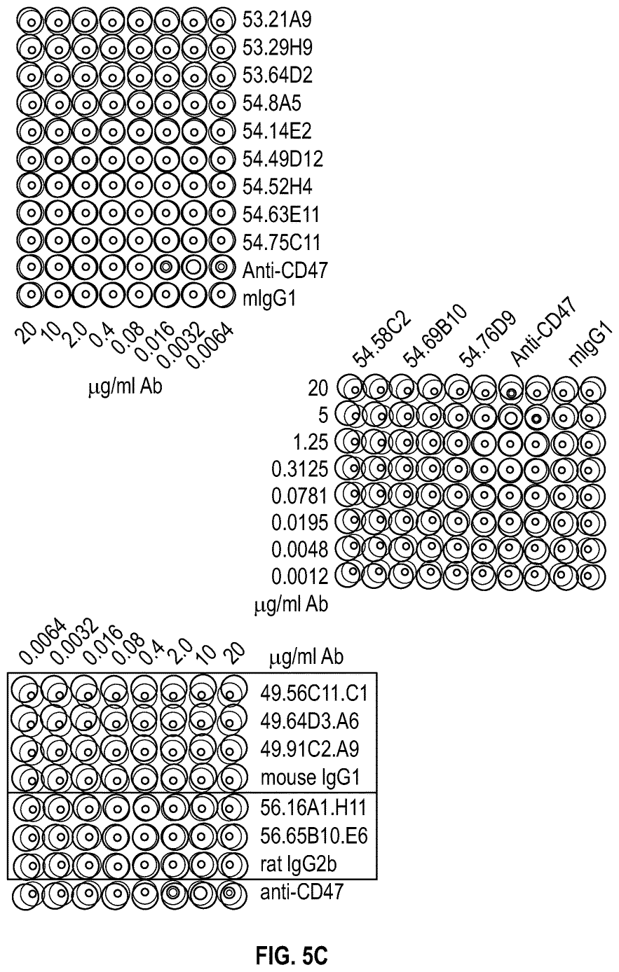

As shown in FIGS. 5A-5C, the selected anti-SIRPα antibodies did not bind to red blood cells (see FIG. 5A) or platelets (see FIG. 5B), and did not aggregate red blood cells (see FIG. 5C).

### Example 5. Anti-tumor Efficacy of Anti-SIRPα Antibodies in a Syngeneic Mouse Tumor Model

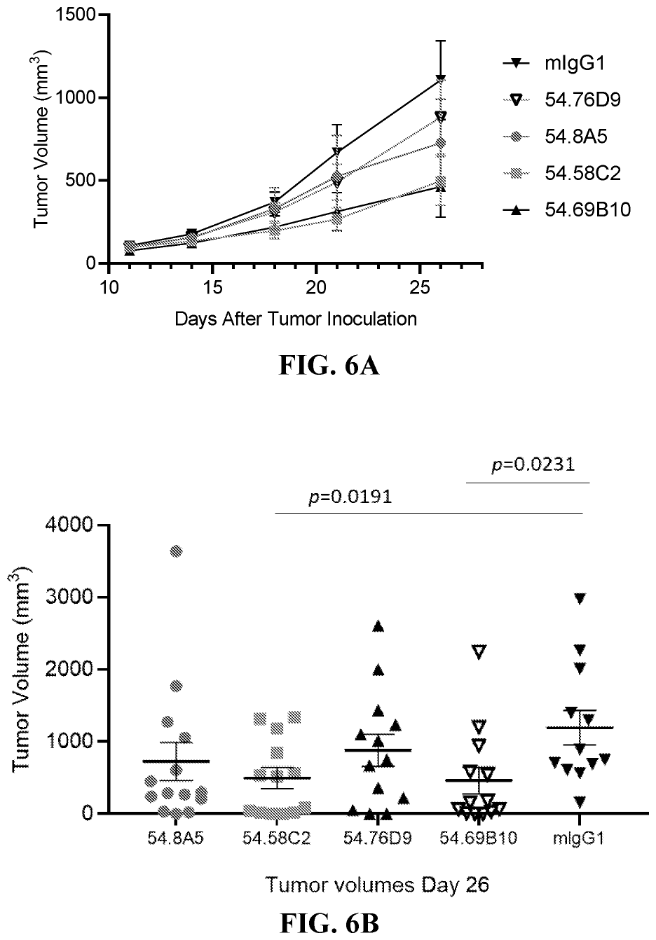

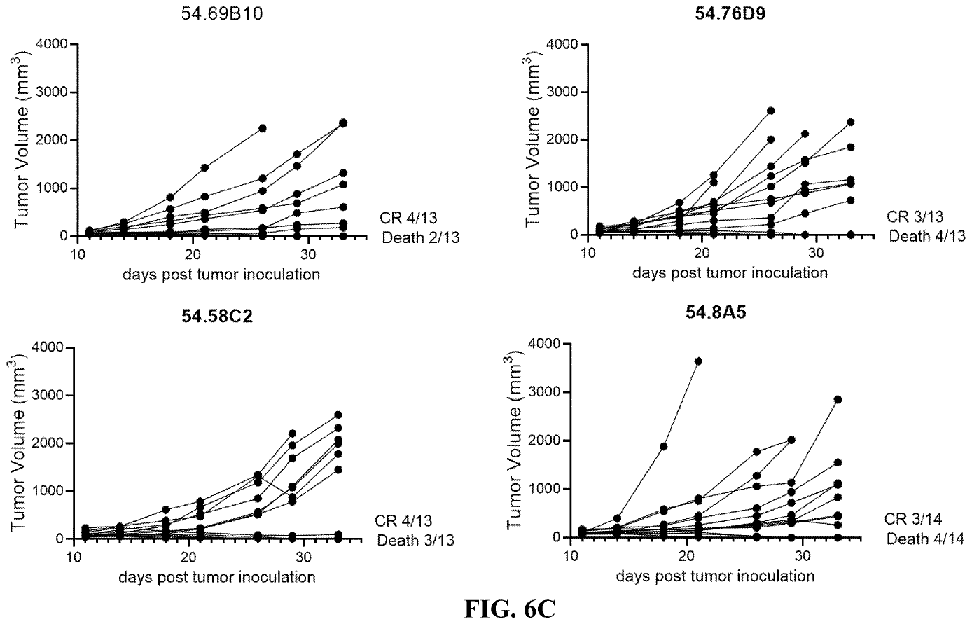

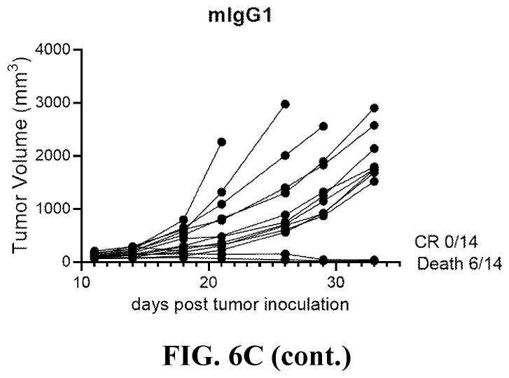

To determine the anti-tumor efficacy of selected anti-SIRPα antibodies in vivo, transgenic double humanized CD47 and SIRPα C57Bl/6 mice (Shanghai Model Organisms), which express human CD47 and SIRPα and have silenced endogenous mouse CD47 and SIRPα expression, were subcutaneously implanted with 1×10⁶ MC38 colon carcinoma cells modified to express human CD47 but not endogenous mouse CD47. Tumor volumes and body weight were monitored starting 7 days post-implantation, and every three days thereafter. 11 days after tumor inoculation, when tumors averaged between 50-100 mm3, mice were randomized into groups of 13 or 14 and treatment with anti-SIRPα antibodies (10 mg/kg) or isotype antibody (Leinco, 10 mg/kg) was initiated. Antibodies were administered every four days for a total of 4 doses. Mice were euthanized when tumor volumes reached 2000 mm3. As shown in FIG. 6A, the selected anti-SIRPα antibodies all reduced mean tumor volumes over time compared to isotype control antibody. As shown in FIG. 6B, anti-SIRPα antibodies 54.69B10 and 54.58C2 demonstrated statistically significant anti-tumor effect. FIG. 6C shows individual tumor growth curves for each treatment group over the duration of the study, with the number of tumor-free animals (complete response, CR). Mice receiving anti-SIRPα antibodies 54.69B10 and 54.58C2 resulted in 4 out 13 tumor free whereas mIgG1 treated animals did not show tumor free (0/14). The number of animals euthanized due to the maximum tumor volume being exceeded also listed. In addition, mice treated with anti-SIRPα antibodies 54.69B10 and 54.58C2 showed less death compared to mIgG1 group.

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

SEQUENCE TABLE

| | | |
|---|---|---|
| SEQ | | |
| ID NO. | Description | Nucleotide or Amino Acid Sequence |

Exemplary anti-SIRPα antibody sequences

| | | |
|---|---|---|
| 1 | 53.21A9.B11 HC CDR1 | SGRYYWS |
| | (Kabat) | |
| 2 | 53.21A9.B11 HC CDR2 | YIYYSGSTNYNPSLKS |
| | (Kabat) | |
| 3 | 53.21A9.B11 HC CDR3 | AYDWNYFFDY |
| | (Kabat) | |
| 4 | 53.21A9.B11 LC CDR1 | RSSQSLLYSNGYNYLE |
| | (Kabat) | |
| 5 | 53.21A9.B11 LC CDR2 | LGSNRAS |
| | (Kabat) | |
| 6 | 53.21A9.B11 LC CDR3 | MQGLQTPIT |
| | (Kabat) | |
| 7 | 53.21A9.B11 VH Amino | QVQLQESGPGLVKPSETLSLTCTVSGGSVSSGRYYWS |
| | acid sequence | WIRQPPGKGLEWIGYIYYSGSTNYNPSLKSRVTISVDT |
| | | SKNQFSLKLTSVTAADTAVYYCARAYDWNYFFDYW |
| | | GQGTLVTVSS |
| 8 | 53.21A9.B11 VL Amino | DIVMTQSPLSLPVTPGEPASISCRSSQSLLYSNGYNYL |
| | acid sequence | EWYLQKPGQSPQLLIYLGSNRASGVPDRFSGSGSGTD |
| | | FTLQISRVEAEDVGVYYCMQGLQTPITFGQGTRLEIK |
| 9 | 53.29H9.C7 HC CDR1 | GYYWS |
| | (Kabat) | |
| 10 | 53.29H9.C7 HC CDR2 | EINHSGSTNYNPSLKR |
| | (Kabat) | |
| 11 | 53.29H9.C7 HC CDR3 | GRGYSGYYYFDY |
| | (Kabat) | |
| 12 | 53.29H9.C7 LC CDR1 | RASQSVSSSYLA |
| | (Kabat) | |
| 13 | 53.29H9.C7 LC CDR2 | GASSRAT |
| | (Kabat) | |
| 14 | 53.29H9.C7 LC CDR3 | QQYDSSPLT |
| | (Kabat) | |
| 15 | 53.29H9.C7 VH Amino | QVQLQQWGAGLLKPSETLSLTCAVYGGSFSGYYWS |
| | acid sequence | WIRQPPGKGLDWIGEINHSGSTNYNPSLKRRVTISIDT |
| | | SKNQFSLKLTSVTAADTAVYYCARGRGYSGYYYFD |
| | | YWGQGTLVTVSS |
| 16 | 53.29H9.C7 VL Amino | EIVLTQSPGTLSLSPGERATLSCRASQSVSSSYLAWYQ |
| | acid sequence | QKPGQAPRLLIYGASSRATGIPDRFSRSGSGTDFTLTIS |
| | | RLEPEDFAVYYCQQYDSSPLTFGGGTKVEIK |
| 17 | 53.64D2.A7 HC CDR1 | SYDMD |
| | (Kabat) | |
| 18 | 53.64D2.A7 HC CDR2 | GIGIAGDTYYPDSVKG |
| | (Kabat) | |
| 19 | 53.64D2.A7 HC CDR3 | GGGWDGSLDY |
| | (Kabat) | |
| 20 | LC CDR1 of 53.64D2.A7, | RASQDINNYLA |
| | 54.8A5.A5, | |
| | 54.58C2.B9.A2, | |
| | 54.63E11.D9, | |
| | 54.69B10.E7, | |
| | 54.75C11.B7, 54.76D9.B6 | |
| | (Kabat) | |
| 21 | LC CDR2 of 53.64D2.A7 | TASRLQS |
| | and 54.8A5.A5 (Kabat) | |
| 22 | LC CDR3 of 53.64D2.A7 | QQYNTYPYT |
| | and 54.63E11.D9 (Kabat) | |
| 23 | 53.64D2.A7 VH Amino | EVQLVESGGGLVQPGGSLRLSCAASGFTFRSYDMDW |
| | acid sequence | VRQATGKGLEWVSGIGIAGDTYYPDSVKGRFTISREN |
| | | AKNSLYLQMNSLRPGDTAVYYCARGGGWDGSLDY |
| | | WGQGTLVTVSS |
| 24 | 53.64D2.A7 VL Amino | DIQMTQSPSSLSASVGDRVTITCRASQDINNYLAWFQ |
| | acid sequence | QKPGRAPKSLIYTASRLQSGVPSKFSGSGSGTDFTLTI |
| | | SSLQPEDFATYYCQQYNTYPYTFGQGTRLEIK |
| 25 | HC CDR1 of 54.8A5.A5, | SYDMH |
| | 54.14E2.A11, | |
| | 54.49D12.A6, | |
| | 54.52H4.B4, 54.54A2.F9, | |
| | 54.58C2.B9.A2, | |
| | 54.63E11.D9, | |
| | 54.69B10.E7, | |
| | 54.75C11.B7, and | |
| | 54.76D9.B6 (Kabat) | |
| 26 | HC CDR2 of 54.8A5.A5, | AIGTAGDTYYPGSVKG |
| | 54.52H4.B4, and | |
| | 54.54A2.F9 (Kabat) | |
| 27 | HC CDR3 of 54.8A5.A5, | GGNWDDAFDI |
| | 54.14E2.A11, | |
| | 54.49D12.A6, | |
| | 54.52H4.B4, 54.54A2.F9, | |
| | 54.58C2.B9.A2, | |
| | 54.63E11.D9, | |
| | 54.69B10.E7, | |
| | 54.75C11.B7 (Kabat) | |
| 28 | 54.8A5.A5 LC CDR3 | QQYTFYPYT |
| | (Kabat) | |
| 29 | 54.8A5.A5 VH Amino | EVQLVESGGDLIQPGGSLRLSCAASGFSFSSYDMHW |
| | acid sequence | VRQTTGKGLEWVSAIGTAGDTYYPGSVKGRFTISRE |
| | | NAKNSLYLQMNSLRAGDTAVYYCARGGNWDDAFDI |
| | | WGQGTVVTVSS |
| 30 | 54.8A5.A5 VL Amino | DIQMTQSPSSLSASVGDRVTITCRASQDINNYLAWFQ |
| | acid sequence | QKPGRAPKSLIFTASRLQSGVPSKFSGSGSGTDFSLTIS |
| | | SLQPEDFATYYCQQYTFYPYTFGQGTKLEIK |
| 31 | 54.14E2.A11 HC CDR2 | VIGIAGDTYYTGSVKG |
| | (Kabat) | |
| 32 | LC CDR1 of | RASQGINNYLA |
| | 54.14E2.A11, | |
| | 54.49D12.A6, | |
| | 54.52H4.B4, and | |
| | 54.54A2.F9 (Kabat) | |
| 33 | LC CDR2 of | TASSLQS |
| | 54.14E2.A11, | |
| | 54.54A2.F9, | |
| | 54.63E11.D9, and | |
| | 54.75C11.B7 (Kabat) | |
| 34 | LC CDR3 of | QQYNSYPYT |
| | 54.14E2.A11, | |
| | 54.49D12.A6, 54.54A2.F9 | |
| | (Kabat) | |
| 35 | 54.14E2.A11 VH Amino | EVQLVESGGGLVQPGGSLRLSCAASGFTFSSYDMHW |
| | acid sequence | VRQATGKGLEWVSVIGIAGDTYYTGSVKGRFTISRK |
| | | NAKNSLYLQMNSLRAGDTAIYYCSRGGNWDDAFDI |
| | | WGQGTMVTVSS |
| 36 | 54.14E2.A11 VL Amino | DIQMTQSPSSLSASVGDRVTITCRASQGINNYLAWFQ |
| | acid sequence | QKPGKAPKSLIYTASSLQSGVPSKFGGSGSGTDFTLTI |
| | | SSLQPEDFATYYCQQYNSYPYTFGQGTKLEIK |
| 37 | 54.49D12.A6 HC CDR2 | VIGTAGDTYFPGSVKG |
| | (Kabat) | |
| 38 | 54.49D12.A6 LC CDR2 | TVSRLQS |
| | (Kabat) | |
| 39 | 54.49D12.A6 VH Amino | EVQLVESGGGLVQPGGSLRLSCAASGFTFSSYDMHW |
| | acid sequence | VRQATGKGLEWVSVIGTAGDTYFPGSVKGRFTISREN |
| | | AKNSLYLQMNSLRAGDTAVYYCVRGGNWDDAFDI |
| | | WGQGTMVTVSS |
| 40 | 54.49D12.A6 VL Amino | DIQMTQSPSSLSASVGDRVTITCRASQGINNYLAWFQ |
| | acid sequence | QKPGKAPKSLIYTVSRLQSGVPSKFSGSGSGTDFTLTI |
| | | SSLQPEDFATYYCQQYNSYPYTFGQGTKLEIK |
| 41 | LC CDR2 of 54.52H4.B4, | TTSSLQS |
| | 54.58C2.B9.A2, | |
| | 54.69B10.E7, and | |
| | 54.76D9.B6 (Kabat) | |
| 42 | 54.52H4.B4 LC CDR3 | QQYISYPYT |
| | (Kabat) | |
| 43 | 54.52H4.B4 VH Amino | EVQLVESGGGLVQPGGSLRLSCAASGFTFSSYDMHW |
| | acid sequence | VRQGTEKGLEWVSAIGTAGDTYYPGSVKGRFTISRE |
| | | NAKNSLFLQMNSLRAGDTAVYYCARGGNWDDAFDI |
| | | WGQGTMVTVSS |
| 44 | 54.52H4.B4 VL Amino | DIQMTQSPSSLSASVGDRVTITCRASQGINNYLAWFQ |
| | acid sequence | QKPGKAPKSLIYTTSSLQSGVPSKFSGSGSGTDYTLTI |
| | | SSLQPEDFATYYCQQYISYPYTFGQGTKLEIK |
| 45 | 54.54A2.F9 VH Amino | EVQLVESGGGLVQPGGSLRLSCAASGFTFSSYDMHW |
| | acid sequence | VRQGTGKGLEWVSAIGTAGDTYYPGSVKGRFTISRE |
| | | NAKNSLFLQMNSLRAGDTAVYYCARGGNWDDAFDI |
| | | WGQGTMVTVSS |
| 46 | 54.54A2.F9 VL Amino | DIQMTQSPSSLSASVGDRVTITCRASQGINNYLAWFQ |
| | acid sequence | QKPGKAPKSLIYTASSLQSGVPSKFSGSGSGTDYTLTI |
| | | SSLQPEDFATYYCQQYNSYPYTFGQGTKLEIK |
| 47 | HC CDR2 of | VIGISGDTYYPGSVKG |
| | 54.58C2.B9.A2, | |
| | 54.63E11.D9, and | |
| | 54.76D9.B6 (Kabat) | |
| 48 | LC CDR3 of | QQYVSYPYT |
| | 54.58C2.B9.A2, | |
| | 54.69B10.E7, and | |
| | 54.76D9.B6 (Kabat) | |
| 49 | VH Amino acid sequence | EVQLVESGGGLVQPGGSLRLSCAASGFTFSSYDMHW |
| | of 54.58C2.B9.A2, and | VRQATGKGLEWVSVIGISGDTYYPGSVKGRFTISREN |
| | 54.63E11.D9 | AKNSLYLQMNSLRAGDTAVYYCARGGNWDDAFDI |
| | | WGQGTMVTVSS |
| 50 | VL Amino acid sequence | DIQMTQSPSSLSASVGDRVTITCRASQDINNYLAWFQ |
| | of 54.58C2.B9.A2, | QKPGKAPKSLIYTTSSLQSGVPSKFSGSGSGTDFTLTIS |
| | 54.69B10.E7, and | SLQPEDFATYYCQQYVSYPYTFGQGTKLEIK |
| | 54.76D9.B6 | |
| 51 | VH Amino acid sequence | EVQLVESGGGLVQPGGSLRLSCAASGFTFSSYDMHW |
| | of 54.63E11.D9 and | VRQATGKGLEWVSVIGISGDTYYPGSVKGRFTISREN |
| | 54.58C2.B9.A2 | AKNSLYLQMNSLRAGDTAVYYCARGGNWDDAFDI |
| | | WGQGTMVTVSS |
| 52 | 54.63E11.D9 VL Amino | DIQMTQSPSSLSASVGDRVTITCRASQDINNYLAWFQ |
| | acid sequence | QKPGKAPKSQIYTASSLQSGVPSKFSGSGSGTDFTLTI |
| | | SSLQPEDFATYYCQQYNTYPYTFGQGTKLEIK |
| 53 | HC CDR2 of | VIGIAGDTYYAGSVKG |
| | 54.69B10.E7 and | |
| | 54.75C11.B7 (Kabat) | |
| 54 | VH Amino acid sequence | EVQLVESGGGLVQPGGSLRLSCAASGFTFSSYDMHW |
| | of 54.69B10.E7 and | VRQTTGKGLEWVSVIGIAGDTYYAGSVKGRFTISREN |
| | 54.75C11.B7 | AKSSLYLQMNSLRAGDTAVYYCARGGNWDDAFDIW |
| | | GQGTMVTVSS |
| 55 | VL Amino acid sequence | DIQMTQSPSSLSASVGDRVTITCRASQDINNYLAWFQ |
| | of 54.69B10.E7, | QKPGKAPKSLIYTTSSLQSGVPSKFSGSGSGTDFTLTIS |
| | 54.58C2.B9.A2, and | SLQPEDFATYYCQQYVSYPYTFGQGTKLEIK |
| | 54.76D9.B6. | |
| 56 | 54.75C11.B7 LC CDR3 | QQYSTYPYT |
| | (Kabat) | |
| 57 | VH Amino acid sequence | EVQLVESGGGLVQPGGSLRLSCAASGFTFSSYDMHW |
| | of 54.75C11.B7 and | VRQTTGKGLEWVSVIGIAGDTYYAGSVKGRFTISREN |
| | 54.69B10.E7 | AKSSLYLQMNSLRAGDTAVYYCARGGNWDDAFDIW |
| | | GQGTMVTVSS |
| 58 | 54.75C11.B7 VL Amino | DIQMTQSPSSLSASVGDRVTITCRASQDINNYLAWFQ |
| | acid sequence | QKPGKAPKSQIYTASSLQSGVPSKFSGSGSGTDFTLTI |
| | | SSLQPEDFATYYCQQYSTYPYTFGQGTKLEIK |
| 59 | 54.76D9.B6 HC CDR3 | GGNWDDALDI |
| | (Kabat) | |
| 60 | VH Amino acid sequence | EVQLVESGGGLVQPGGSLRLSCAASGFTFSSYDMHW |
| | of 54.76D9.B6 and | VRQATGKGLEWVSVIGISGDTYYPGSVKGRFTISREN |
| | 54.75C11.B7 | AKNSLHLQMNSLRAGDTAVYYCARGGNWDDALDI |
| | | WGQGTMVTVSS |
| 61 | VL Amino acid sequence | DIQMTQSPSSLSASVGDRVTITCRASQDINNYLAWFQ |
| | of 54.76D9.B6, | QKPGKAPKSLIYTTSSLQSGVPSKFSGSGSGTDFTLTIS |
| | 54.58C2.B9.A2, and | SLQPEDFATYYCQQYVSYPYTFGQGTKLEIK |
| | 54.69B10.E7 | |
| 62 | Group C consensus | SYDMX₁ |
| | sequence - CDRH1 | X₁ = D or H |
| 63 | Group C consensus | X₁IGX₂X₃GDTYX₄X₅GSVKG |
| | sequence - CDRH2 | X₁ = A or V |
| | | X₂ = I or T |
| | | X₃ = A or S |
| | | X₄ = Y or F |
| | | X₅ = A, P, or T |
| 64 | Group C consensus | GGNWDDAX₁DI |
| | sequence - CDRH3 | X₁ = D or L |
| 65 | Group C consensus | RASQX₁INNYLA |
| | sequence - CDRL1 | X₁ = D or G |
| 66 | Group C consensus | TX₁SX₂LQS |
| | sequence - CDRL2 | X₁ = A, V or T |
| | | X₂ = R or S |
| 67 | Group C consensus | QQYX₁X₂YPYT |
| | sequence - CDRL3 | X₁ = I, N, T, V or S |
| | | X₂ = F, T or S |
| 68 | Linker | (G)ₙ |
| | | n>=1 |
| 69 | Linker | (GS)ₙ |
| | | 8>=n>=1 |
| 70 | Linker | (GSGGS)ₙ |
| | | 8>=n>=1 |
| 71 | Linker | (GGGGS)ₙ |
| | | 8>=n>=1 |
| 72 | Linker | (GGGS)ₙ |
| | | 8>=n>=1 |
| 73 | Linker | (GGGGS)₃ |
| 74 | Linker | (GGGGS)₆ |
| 75 | Linker | (GSTSGSGKPGSGEGS)ₙ |
| | | 3>=n>=1 |
| 76 | Linker | (GGGS)ₙ |
| | | 8>=n>=1 |
| 77 | Human SIRPα | MEPAGPAPGRLGPLLCLLLAASCAWSGVAGEEELQV |
| | UniProtKB/Swiss-Prot: | IQPDKSVLVAAGETATLRCTATSLIPVGPIQWFRGAG |
| | P78324 | PGRELIYNQKEGHFPRVTTVSDLTKRNNMDFSIRIGN |
| | | ITPADAGTYYCVKFRKGSPDDVEFKSGAGTELSVRA |
| | | KPSAPVVSGPAARATPQHTVSFTCESHGFSPRDITLK |
| | | WFKNGNELSDFQTNVDPVGESVSYSIHSTAKVVLTR |
| | | EDVHSQVICEVAHVTLQGDPLRGTANLSETIRVPPTL |
| | | EVTQQPVRAENQVNVTCQVRKFYPQRLQLTWLENG |
| | | NVSRTETASTVTENKDGTYNWMSWLLVNVSAHRD |
| | | DVKLTCQVEHDGQPAVSKSHDLKVSAHPKEQGSNT |
| | | AAENTGSNERNIYIVVGVVCTLLVALLMAALYLVRI |
| | | RQKKAQGSTSSTRLHEPEKNAREITQDTNDITYADL |
| | | NLPKGKKPAPQAAEPNNHTEYASIQTSPQPASEDTLT |
| | | YADLDMVHLNRTPKQPAPKPEPSFSEYASVQVPRK |
| 78 | 49.56C11.C1 HC CDR1 | NHAMS |
| | (Kabat) | |
| 79 | 49.56C11.C1 HC CDR2 | TFGSGGSYTYYLDSVKG |
| | (Kabat) | |
| 80 | HC CDR3 of | SGWDGWFAY |
| | 49.56C11.C1 and | |
| | 49.64D3.A6 (Kabat) | |
| 81 | 49.56C11.C1 LC CDR1 | KSSQSLLNSNNQKNYLA |
| | (Kabat) | |
| 82 | LC CDR2 of | FASTRES |
| | 49.56C11.C1 and | |
| | 49.64D3.A6 (Kabat) | |
| 83 | 49.56C11.C1 LC CDR3 | QQHYSTLPWT |
| | (Kabat) | |
| 84 | 49.56C11.C1 VH Amino | EVMLVESGGGLVKPGGSLKLTCAASGFTFSNHAMS |
| | acid sequence | WVRQTPEKRLEWVATFGSGGSYTYYLDSVKGRFTVS |
| | | RDNAKNTLYLQMSSLRSEDTAMYYCSRSGWDGWFA |
| | | YWGQGTLVTVSA |
| 85 | 49.56C11.C1 VL Amino | DIVMTQSPSSLAMSIGQKVTMNCKSSQSLLNSNNQK |
| | acid sequence | NYLAWYQQKPGQSPKLLIYFASTRESGVPDRFIGSGS |
| | | GTDFTLTISSVQAEDLANYFCQQHYSTLPWTFGGGTK |
| | | LEIK |
| 86 | 49.64D3.A6 HC CDR1 | NYAMS |
| | (Kabat) | |
| 87 | 49.64D3.A6 HC CDR2 | TFSSGGSYTYYQDSVKG |
| | (Kabat) | |
| 88 | 49.64D3.A6 LC CDR1 | KSSQSLLNSSNQKNYLA |
| | (Kabat) | |
| 89 | 49.64D3.A6 LC CDR3 | QQHCSTLPWT |
| | (Kabat) | |
| 90 | 49.64D3.A6 VH Amino | EVMLVESGGGLVKPGGSLKLTCAASGFIFSNYAMSW |
| | acid sequence | VRQTPEKRLEWVATFSSGGSYTYYQDSVKGRFTVSR |
| | | DNAKNTLYLQMSSLRSEDTAMYYCARSGWDGWFA |
| | | YWGQGTLVTVSA |
| 91 | 49.64D3.A6 VL Amino | DIVMTQSPSSLAMSVGQKVTMSCKSSQSLLNSSNQK |
| | acid sequence | NYLAWYQQKPGQSPKLLVYFASTRESGVPDRFIGSG |
| | | SGTDFTLTISSVQAEDLANYFCQQHCSTLPWTFGGGT |
| | | KLEIK |
| 92 | 49.91C2.A9 HC CDR1 | RYWMS |
| | (Kabat) | |
| 93 | 49.91C2.A9 HC CDR2 | EINPDSSTINYTPSLKD |
| | (Kabat) | |
| 94 | 49.91C2.A9 HC CDR3 | SFYGSSYWYFDV |
| | (Kabat) | |
| 95 | 49.91C2.A9 LC CDR1 | RASESVDNYGISFMN |
| | (Kabat) | |
| 96 | 49.91C2.A9 LC CDR2 | AASNQGS |
| | (Kabat) | |
| 97 | 49.91C2.A9 LC CDR3 | QQSKEVPYT |
| | (Kabat) | |
| 98 | 49.91C2.A9 VH Amino | EVKLLESGGGLVQPGGSLKLSCAASGFDFSRYWMS |
| | acid sequence | WVRQAPGKGLEWIGEINPDSSTINYTPSLKDKFIISRD |
| | | NAKNTLYLQMSKVRSEDTALYYCATSFYGSSYWYF |
| | | DVWGAGTTVTVSS |
| 99 | 49.91C2.A9 VL Amino | DIVLTQSPASLAVSLGQRATISCRASESVDNYGISFMN |
| | acid sequence | WFQQKPGQPPKVLIYAASNQGSGVPARFSGSGSGTD |
| | | FSLNIHPMEEDDTAMYFCQQSKEVPYTFGGGTKLEIK |
| 100 | 56.16A1.H11 HC CDR1 | NYDMA |
| | (Kabat) | |
| 101 | 56.16A1.H11 HC CDR2 | SISYGGSRIYYRDSVKG |
| | (Kabat) | |
| 102 | 56.16A1.H11 HC CDR3 | DYGYNPSYYWYFDF |
| | (Kabat) | |
| 103 | 56.16A1.H11 LC CDR1 | RASQGISNYLN |
| | (Kabat) | |
| 104 | 56.16A1.H11 LC CDR2 | YTSNLQS |
| | (Kabat) | |
| 105 | 56.16A1.H11 LC CDR3 | QQYDSSPYT |
| | (Kabat) | |
| 106 | 56.16A1.H11 VH Amino | EVQLVESGGGLVQPGRSMKLSCAASGFIFTNYDMAW |
| | acid sequence | VRQAPTKGLEWVASISYGGSRIYYRDSVKGRFTISRD |
| | | DAKSTLYLQMDSLRSEDTATYYCTTDYGYNPSYYW |
| | | YFDFWGPGTMVTVSS |
| 107 | 56.16A1.H11 VL Amino | DIQMTQTPSSMPASLGERVTISCRASQGISNYLNWYQ |
| | acid sequence | QKPDGTIKPLIYYTSNLQSGVPSRFSGSGSGTDYSLTIS |
| | | SLEPEDFAMYYCQQYDSSPYTFGAGTKLELKR |
| 108 | Group D consensus | NX₁AMS |
| | sequence - CDRH1 | X₁ = Hor Y |
| 109 | Group D consensus | TFX1SGGSYTYYX2DSVKG |
| | sequence - CDRH2 | X₁ = G or S |
| | | X₂ = L or Q |
| 110 | Group D consensus | KSSQSLLNSXINQKNYLA |
| | sequence - CDRL1 | X₁ = N or S |
| 111 | Group D consensus | QQHX1STLPWT |
| | sequence - CDRL3 | X₁ = Y or C |

What is claimed:

1. An anti-SIRPα construct comprising an antibody moiety comprising a heavy chain variable region (V_{H}) and a light chain variable region (V_{L}), wherein:
1) the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3, or a variant thereof comprising up to 5, 4, 3, 2, or 1 amino acid substitutions in the HC-CDRs; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6, or a variant thereof comprising up to 5, 4, 3, 2, or 1 amino acid substitutions in the LC-CDRs;
2) the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11, or a variant thereof comprising up to 5, 4, 3, 2, or 1 amino acid substitutions in the HC-CDRs; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14, or a variant thereof comprising up to 5, 4, 3, 2, or 1 amino acid substitutions in the LC-CDRs;
3) the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 22, or a variant thereof comprising up to 5, 4, 3, 2, or 1 amino acid substitutions in the LC-CDRs;
4) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28, or a variant thereof comprising up to 5, 4, 3, 2, or 1 amino acid substitutions in the LC-CDRs;
5) the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs;
6) the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34, or a variant thereof comprising up to 5, 4, 3, 2, or 1 amino acid substitutions in the LC-CDRs;
7) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42, or a variant thereof comprising up to 5, 4, 3, 2, or 1 amino acid substitutions in the LC-CDRs;
8) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34, or a variant thereof comprising up to 5, 4, 3, 2, or 1 amino acid substitutions in the LC-CDRs;
9) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs;
10) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22, or a variant thereof comprising up to 5, 4, 3, 2, or 1 amino acid substitutions in the LC-CDRs;
11) the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs;
12) the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56, or a variant thereof comprising up to 5, 4, 3, 2, or 1 amino acid substitutions in the LC-CDRs;
13) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59, or a variant thereof comprising up to 5, 4, 3, 2, or 1 amino acid substitutions in the HC-CDRs; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48, or a variant thereof comprising up to 5, 4, 3, 2, or 1 amino acid substitutions in the LC-CDRs;
14) the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6;
15) the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14;
16) the V_{H} comprises a HC-CDR1 comprising the amino acid sequence of SEQ ID NO: 62, a HC-CDR2 comprising the amino acid sequence of SEQ ID NO: 63 or 18, and a HC-CDR3 comprising the amino acid sequence of SEQ ID NO: 64 or 19; and the V_{L} comprises a LC-CDR1 comprising the amino acid sequence of SEQ ID NO: 65, a LC-CDR2 comprising the amino acid sequence of SEQ ID NO: 66, and a LC-CDR3 comprising the amino acid sequence of SEQ ID NO: 67;
17) the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83, or a variant thereof comprising up to 5, 4, 3, 2, or 1 amino acid substitutions in the LC-CDRs;
18) the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80, or a variant thereof comprising up to 5, 4, 3, 2, or 1 amino acid substitutions in the HC-CDRs; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89, or a variant thereof comprising up to 5, 4, 3, 2, or 1 amino acid substitutions in the LC-CDRs;
19) the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94, or a variant thereof comprising up to 5, 4, 3, 2, or 1 amino acid substitutions in the HC-CDRs; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97, or a variant thereof comprising up to 5, 4, 3, 2, or 1 amino acid substitutions in the LC-CDRs;
20) the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102, or a variant thereof comprising up to 5, 4, 3, 2, or 1 amino acid substitutions in the HC-CDRs; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105, or a variant thereof comprising up to 5, 4, 3, 2, or 1 amino acid substitutions in the LC-CDRs;
21) the V_{H} comprises a HC-CDR1 comprising the amino acid sequence of SEQ ID NO: 108, a HC-CDR2 comprising the amino acid sequence of SEQ ID NO: 109, and a HC-CDR3 comprising the amino acid sequence of SEQ ID NO: 80; and the V_{L} comprises a LC-CDR1 comprising the amino acid sequence of SEQ ID NO: 110, a LC-CDR2 comprising the amino acid sequence of SEQ ID NO: 82, and a LC-CDR3 comprising the amino acid sequence of SEQ ID NO: 111;
22) the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97; or
23) the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105.

2. An anti-SIRPα construct comprising an antibody moiety that specifically binds to SIRPα, comprising:
1) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 7, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 8;
2) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 15, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 16;
3) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 23, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 24;
4) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 29, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 30;
5) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 35, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 36;
6) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 39, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 40;
7) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 43, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 44;
8) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 45, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 46;
9) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 49, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 50;
10) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 51, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 52;
11) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 54, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 55;
12) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 57, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 58;
13) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 60, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 61;
14) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 84, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 85;
15) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 90, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 91;
16) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 98, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 99; or
17) a HC-CDR1, a HC-CDR2, and a HC-CDR3, respectively comprising the amino acid sequences of a CDR1, a CDR2, and a CDR3 within a V_{H} comprising the amino acid sequence set forth in SEQ ID NO: 106, and a LC-CDR1, a LC-CDR2, and a LC-CDR3, respectively comprising the amino acid sequences of a CDR1, a CDR2, and a CDR3 within a V_{L} comprising the amino acid sequence set forth in SEQ ID NO: 107.

3. The anti-SIRPα construct of claim 1 or claim 2, wherein
1) the V_{H} comprises a HC-CDR1 comprising the amino acid sequence of SEQ ID NO: 1, a HC-CDR2 comprising the amino acid sequence of SEQ ID NO: 2, and a HC-CDR3 comprising the amino acid sequence of SEQ ID NO: 3; and the V_{L} comprises a LC-CDR1 comprising the amino acid sequence of SEQ ID NO: 4, a LC-CDR2 comprising the amino acid sequence of SEQ ID NO: 5, and a LC-CDR3 comprising the amino acid sequence of SEQ ID NO: 6;
2) the V_{H} comprises a HC-CDR1 comprising the amino acid sequence of SEQ ID NO: 9, a HC-CDR2 comprising the amino acid sequence of SEQ ID NO: 10, and a HC-CDR3 comprising the amino acid sequence of SEQ ID NO: 11; and the V_{L} comprises a LC-CDR1 comprising the amino acid sequence of SEQ ID NO: 12, a LC-CDR2 comprising the amino acid sequence of SEQ ID NO: 13, and a LC-CDR3 comprising the amino acid sequence of SEQ ID NO: 14;
3) the V_{H} comprises a HC-CDR1 comprising the amino acid sequence of SEQ ID NO: 17, a HC-CDR2 comprising the amino acid sequence of SEQ ID NO: 18, and a HC-CDR3 comprising the amino acid sequence of SEQ ID NO: 19; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 22;
4) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 21, and a LC-CDR3 comprising the amino acid sequence of SEQ ID NO: 28;
5) the V_{H} comprises a HC-CDR1 comprising the amino acid sequence of SEQ ID NO: 25, a HC-CDR2 comprising the amino acid sequence of SEQ ID NO: 31, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34;
6) the V_{H} comprises a HC-CDR1 comprising the amino acid sequence of SEQ ID NO: 25, a HC-CDR2 comprising the amino acid sequence of SEQ ID NO: 37, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 38, and a LC-CDR3 comprising the amino acid sequence of SEQ ID NO: 34;
7) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 41, and a LC-CDR3 comprising the amino acid sequence of SEQ ID NO: 42;
8) the V_{H} comprises a HC-CDR1 comprising the amino acid sequence of SEQ ID NO: 25, a HC-CDR2 comprising the amino acid sequence of SEQ ID NO: 26, and a HC-CDR3 comprising the amino acid sequence of SEQ ID NO: 27; and the V_{L} comprises a LC-CDR1 comprising the amino acid sequence of SEQ ID NO: 32, a LC-CDR2 comprising the amino acid sequence of SEQ ID NO: 33, and a LC-CDR3 comprising the amino acid sequence of SEQ ID NO: 34;
9) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27 and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48;
10) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 22;
11) the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48;
12) the V_{H} comprises a HC-CDR1 comprising the amino acid sequence of SEQ ID NO: 25, a HC-CDR2 comprising the amino acid sequence of SEQ ID NO: 53, and a HC-CDR3 comprising the amino acid sequence of SEQ ID NO: 27; and the V_{L} comprises a LC-CDR1 comprising the amino acid sequence of SEQ ID NO: 20, a LC-CDR2 comprising the amino acid sequence of SEQ ID NO: 33, and a LC-CDR3 comprising the amino acid sequence of SEQ ID NO: 56;
13) the V_{H} comprises a HC-CDR1 comprising the amino acid sequence of SEQ ID NO: 25, a HC-CDR2 comprising the amino acid sequence of SEQ ID NO: 47, and a HC-CDR3 comprising the amino acid sequence of SEQ ID NO: 59; and the V_{L} comprises a LC-CDR1 comprising the amino acid sequence of SEQ ID NO: 20, a LC-CDR2 comprising the amino acid sequence of SEQ ID NO: 41, and a LC-CDR3 comprising the amino acid sequence of SEQ ID NO: 48;
14) the V_{H} comprises a HC-CDR1 comprising the amino acid sequence of SEQ ID NO: 78, a HC-CDR2 comprising the amino acid sequence of SEQ ID NO: 79, and a HC-CDR3 comprising the amino acid sequence of SEQ ID NO: 80; and the V_{L} comprises a LC-CDR1 comprising the amino acid sequence of SEQ ID NO: 81, a LC-CDR2 comprising the amino acid sequence of SEQ ID NO: 82, and a LC-CDR3 comprising the amino acid sequence of SEQ ID NO: 83;
15) the V_{H} comprises a HC-CDR1 comprising the amino acid sequence of SEQ ID NO: 86, a HC-CDR2 comprising the amino acid sequence of SEQ ID NO: 87, and a HC-CDR3 comprising the amino acid sequence of SEQ ID NO: 80; and the V_{L} comprises a LC-CDR1 comprising the amino acid sequence of SEQ ID NO: 88, a LC-CDR2 comprising the amino acid sequence of SEQ ID NO: 82, and a LC-CDR3 comprising the amino acid sequence of SEQ ID NO: 89;
16) the V_{H} comprises a HC-CDR1 comprising the amino acid sequence of SEQ ID NO: 92, a HC-CDR2 comprising the amino acid sequence of SEQ ID NO: 93, and a HC-CDR3 comprising the amino acid sequence of SEQ ID NO: 94; and the V_{L} comprises a LC-CDR1 comprising the amino acid sequence of SEQ ID NO: 95, a LC-CDR2 comprising the amino acid sequence of SEQ ID NO: 96, and a LC-CDR3 comprising the amino acid sequence of SEQ ID NO: 97; or
17) the V_{H} comprises a HC-CDR1 comprising the amino acid sequence of SEQ ID NO: 100, a HC-CDR2 comprising the amino acid sequence of SEQ ID NO: 101, and a HC-CDR3 comprising the amino acid sequence of SEQ ID NO: 102; and the V_{L} comprises a LC-CDR1 comprising the amino acid sequence of SEQ ID NO: 103, a LC-CDR2 comprising the amino acid sequence of SEQ ID NO: 104, and a LC-CDR3 comprising the amino acid sequence of SEQ ID NO: 105.

4. The anti-SIRPα construct of any one of claims 1-3, wherein the V_{H} comprises an amino acid sequence of any one of SEQ ID NOs: 7, 15, 23, 29, 35, 39, 43, 45, 49, 51, 54, 57, 60, 84, 90, 98 and 106, or a variant comprising an amino acid sequence having at least about 80% sequence identity; and/or wherein the V_{L} comprises an amino acid sequence of any one of SEQ ID NOs: 8, 16, 24, 30, 36, 40, 44, 46, 50, 52, 55, 58, 61, 85, 91, 99 and 107, or a variant comprising an amino acid sequence having at least about 80% sequence identity.

5. The anti-SIRPα construct of any one of claims 1-4, wherein:
1) the V_{H} comprises the amino acid sequence of SEQ ID NO: 7, or a variant comprising an amino acid sequence having at least about 80% sequence identity; and the V_{L} comprises the amino acid sequence of SEQ ID NO: 8, or a variant comprising an amino acid sequence having at least about 80% sequence identity,
2) the V_{H} comprises the amino acid sequence of SEQ ID NO: 15, or a variant comprising an amino acid sequence having at least about 80% sequence identity; and the V_{L} comprises the amino acid sequence of SEQ ID NO: 16, or a variant comprising an amino acid sequence having at least about 80% sequence identity,
3) the V_{H} comprises the amino acid sequence of SEQ ID NO: 23, or a variant comprising an amino acid sequence having at least about 80% sequence identity; and the V_{L} comprises the amino acid sequence of SEQ ID NO: 24, or a variant comprising an amino acid sequence having at least about 80% sequence identity,
4) the V_{H} comprises the amino acid sequence of SEQ ID NO: 29, or a variant comprising an amino acid sequence having at least about 80% sequence identity; and the V_{L} comprises the amino acid sequence of SEQ ID NO: 30, or a variant comprising an amino acid sequence having at least about 80% sequence identity,
5) the V_{H} comprises the amino acid sequence of SEQ ID NO: 35, or a variant comprising an amino acid sequence having at least about 80% sequence identity; and the V_{L} comprises the amino acid sequence of SEQ ID NO: 36, or a variant comprising an amino acid sequence having at least about 80% sequence identity,
6) the V_{H} comprises the amino acid sequence of SEQ ID NO: 39, or a variant comprising an amino acid sequence having at least about 80% sequence identity; and the V_{L} comprises the amino acid sequence of SEQ ID NO: 40, or a variant comprising an amino acid sequence having at least about 80% sequence identity,
7) the V_{H} comprises the amino acid sequence of SEQ ID NO: 43, or a variant comprising an amino acid sequence having at least about 80% sequence identity; and the V_{L} comprises the amino acid sequence of SEQ ID NO: 44, or a variant comprising an amino acid sequence having at least about 80% sequence identity,
8) the V_{H} comprises an amino acid sequence of SEQ ID NO: 45, or a variant comprising an amino acid sequence having at least about 80% sequence identity; and the V_{L} comprises an amino acid sequence of SEQ ID NO: 46, or a variant comprising an amino acid sequence having at least about 80% sequence identity,
9) the V_{H} comprises the amino acid sequence of SEQ ID NO: 49, or a variant comprising an amino acid sequence having at least about 80% sequence identity; and the V_{L} comprises the amino acid sequence of SEQ ID NO: 50, or a variant comprising an amino acid sequence having at least about 80% sequence identity,
10) the V_{H} comprises the amino acid sequence of SEQ ID NO: 51, or a variant comprising an amino acid sequence having at least about 80% sequence identity; and the V_{L} comprises the amino acid sequence of SEQ ID NO: 52, or a variant comprising an amino acid sequence having at least about 80% sequence identity,
11) the V_{H} comprises the amino acid sequence of SEQ ID NO: 54, or a variant comprising an amino acid sequence having at least about 80% sequence identity; and the V_{L} comprises the amino acid sequence of SEQ ID NO: 55, or a variant comprising an amino acid sequence having at least about 80% sequence identity,
12) the V_{H} comprises the amino acid sequence of SEQ ID NO: 57, or a variant comprising an amino acid sequence having at least about 80% sequence identity; and the V_{L} comprises the amino acid sequence of SEQ ID NO: 58, or a variant comprising an amino acid sequence having at least about 80% sequence identity,
13) the V_{H} comprises the amino acid sequence of SEQ ID NO: 60, or a variant comprising an amino acid sequence having at least about 80% sequence identity; and the V_{L} comprises the amino acid sequence of SEQ ID NO: 61, or a variant comprising an amino acid sequence having at least about 80% sequence identity,
14) the V_{H} comprises the amino acid sequence of SEQ ID NO: 84, or a variant comprising an amino acid sequence having at least about 80% sequence identity; and the V_{L} comprises the amino acid sequence of SEQ ID NO: 85, or a variant comprising an amino acid sequence having at least about 80% sequence identity,
15) the V_{H} comprises the amino acid sequence of SEQ ID NO: 90, or a variant comprising an amino acid sequence having at least about 80% sequence identity; and the V_{L} comprises the amino acid sequence of SEQ ID NO: 91, or a variant comprising an amino acid sequence having at least about 80% sequence identity,
16) the V_{H} comprises the amino acid sequence of SEQ ID NO: 98, or a variant comprising an amino acid sequence having at least about 80% sequence identity; and the V_{L} comprises the amino acid sequence of SEQ ID NO: 99, or a variant comprising an amino acid sequence having at least about 80% sequence identity,
17) the V_{H} comprises the amino acid sequence of SEQ ID NO: 106, or a variant comprising an amino acid sequence having at least about 80% sequence identity; and the V_{L} comprises the amino acid sequence of SEQ ID NO: 107, or a variant comprising an amino acid sequence having at least about 80% sequence identity.

6. The anti-SIRPα construct of any one of claims 1-5, wherein:
1) the V_{H} comprises the amino acid sequence set forth in SEQ ID NO: 7, and the V_{L} comprises the amino acid sequence set forth in SEQ ID NO: 8;
2) the V_{H} comprises the amino acid sequence set forth in SEQ ID NO: 15, and the V_{L} comprises the amino acid sequence set forth in SEQ ID NO: 16;
3) the V_{H} comprises the amino acid sequence set forth in SEQ ID NO: 23, and the V_{L} comprises the amino acid sequence set forth in SEQ ID NO: 24;
4) the V_{H} comprises the amino acid sequence set forth in SEQ ID NO: 29, and the V_{L} comprises the amino acid sequence set forth in SEQ ID NO: 30;
5) the V_{H} comprises the amino acid sequence set forth in SEQ ID NO: 35, and the V_{L} comprises the amino acid sequence set forth in SEQ ID NO: 36;
6) the V_{H} comprises the amino acid sequence set forth in SEQ ID NO: 39, and the V_{L} comprises the amino acid sequence set forth in SEQ ID NO: 40;
7) the V_{H} comprises the amino acid sequence set forth in SEQ ID NO: 43, and the V_{L} comprises the amino acid sequence set forth in SEQ ID NO: 44;
8) the V_{H} comprises the amino acid sequence set forth in SEQ ID NO: 45, and the V_{L} comprises the amino acid sequence set forth in SEQ ID NO: 46;
9) the V_{H} comprises the amino acid sequence set forth in SEQ ID NO: 49, and the V_{L} comprises the amino acid sequence set forth in SEQ ID NO: 50;
10) the V_{H} comprises the amino acid sequence set forth in SEQ ID NO: 51, and the V_{L} V_{L} comprises the amino acid sequence set forth in SEQ ID NO: 52;
11) the V_{H} comprises the amino acid sequence set forth in SEQ ID NO: 54, and the V_{L} comprises the amino acid sequence set forth in SEQ ID NO: 55;
12) the V_{H} comprises the amino acid sequence set forth in SEQ ID NO: 57, and the V_{L} comprises the amino acid sequence set forth in SEQ ID NO: 58;
13) the V_{H} comprises the amino acid sequence set forth in SEQ ID NO: 60, and the V_{L} comprises the amino acid sequence set forth in SEQ ID NO: 61;
14) the V_{H} comprises the amino acid sequence set forth in SEQ ID NO: 84, and the V_{L} comprises the amino acid sequence set forth in SEQ ID NO: 85;
15) the V_{H} comprises the amino acid sequence set forth in SEQ ID NO: 90, and the V_{L} comprises the amino acid sequence set forth in SEQ ID NO: 91;
16) the V_{H} comprises the amino acid sequence set forth in SEQ ID NO: 98, and the V_{L} comprises the amino acid sequence set forth in SEQ ID NO: 99; or
17) the V_{H} comprises the amino acid sequence set forth in SEQ ID NO: 106, and the V_{L} comprises the amino acid sequence set forth in SEQ ID NO: 107.

7. The anti-SIRPα construct of any one of claims 1-6, wherein the antibody moiety is an antibody or an antigen-binding fragment thereof.

8. The anti-SIRPα construct of claim 7, wherein the antigen-binding fragment is selected from the group consisting of a full-length antibody, a bispecific antibody, a single-chain Fv (scFv) fragment, a Fab fragment, a Fab′ fragment, a F(ab′)₂, an Fv fragment, a disulfide stabilized Fv fragment (dsFv), a (dsFv)₂, a Fv-Fc fusion, a scFv-Fc fusion, a scFv-Fv fusion, a diabody, a tribody, and a tetrabody.

9. The anti-SIRPα construct of claim 7, wherein the antibody moiety is a full-length antibody.

10. The anti-SIRPα construct of any one of claims 1-9, wherein the antibody moiety has an Fc fragment selected from the group consisting of Fc fragments form IgG, IgA, IgD, IgE, IgM, and combinations and hybrids thereof.

11. The anti-SIRPα construct of claim 10, wherein the Fc fragment is selected from the group consisting of Fc fragments from IgG1, IgG2, IgG3, IgG4, and combinations and hybrids thereof.

12. The anti-SIRPα construct of claim 10 or 11, wherein the Fc fragment has a reduced effector function as compared to the corresponding wildtype Fc fragment.

13. The anti-SIRPα construct of claim 10 or claim 11, wherein the Fc fragment has an enhanced effector function as compared to the corresponding wildtype Fc fragment.

14. The anti-SIRPα construct of any one of claims 1-13, wherein the construct is a full-length antibody, a fusion protein, or an immunoconjugate.

15. The anti-SIRPα construct of any one of claims 1-14, which is conjugated or recombinantly fused to a diagnostic agent, detectable agent or therapeutic agent.

16. The anti-SIRPα construct of claim 15, wherein the therapeutic agent is a chemotherapeutic agent, cytotoxin, or drug.

17. The anti-SIRPα construct of any one of claims 1-16, wherein the SIRPα is a human SIRPα.

18. An anti-SIRPα construct competes for a binding epitope of SIRPα with the anti-SIRPα construct of any one of claims 1-17.

19. An anti-SIRPα construct that binds to essentially the same epitope as the anti-SIRPα construct of any one of claims 1-17.

20. A pharmaceutical composition comprising the anti-SIRPα construct of any one of claims 1-19, and a pharmaceutical acceptable carrier.

21. A nucleic acid encoding the anti-SIRPα construct of any one of claims 1-19 or a portion thereof.

22. A vector comprising the nucleic acid of claim 21.

23. A host cell comprising the nucleic acid of claim 21, or the vector of claim 22.

24. A method of producing an anti-SIRPα construct comprising:
a) culturing the host cell of claim 23 under conditions effective to express the anti-SIRPα construct; and
b) obtaining the expressed anti-SIRPα construct from the cell.

25. A method of treating a disease or condition in an individual, comprising administering to the individual an effective mount of the anti-SIRPα construct of any one of claims 1-19, or the pharmaceutical composition of claim 20.

26. The method of claim 25, wherein the disease or condition is a tumor.

27. The method of claim 26, wherein the tumor is cancer.

28. The method of claim 26 or 27, wherein the tumor is a solid tumor.

29. The method of any one of claims 26-28, wherein the tumor is an advanced or malignant tumor.

30. The method of any one of claims 26-29, wherein the tumor has an increased expression level of SIRPα.

31. The method of any one of claims 26-30, wherein the tumor is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer, and melanoma.

32. The method of claim 25, wherein the disease or condition is a viral infection.

33. The method of claim 32, wherein the expression level of SIRPα at an infected site is higher than that of an uninfected site.

34. The method of any one of claims 25-33, wherein the method further comprises administering to the individual a second agent.

35. The method of claim 34, wherein the second agent is selected from the group consisting of a chemotherapeutic agent, an immunomodulator, an anti-angiogenesis agent, a growth inhibitory agent, and an antineoplastic agent.

36. The method of claim 34, wherein the second agent comprises a cell comprising a chimeric antigen receptor that specifically binds to a tumor antigen.

37. The method of any one of claims 34-36, wherein the anti-SIRPα construct and the second agent are administered simultaneously or concurrently.

38. The method of any one of claims 34-37, wherein the anti-SIRPα construct and the second agent are administered sequentially.

39. The method of any one of claims 34-38, wherein the anti-SIRPα construct and/or the second agent are administered parentally.

40. The method of any one of claims 25-39, wherein the anti-SIRPα construct is administered to a tumor tissue or infection site directly.

41. The method of any one of claims 25-40, wherein the anti-SIRPα construct is administered at a dose of about 0.001 μg/kg to about 100 mg/kg.

42. The method of any one of claims 25-41, wherein the individual has an increased number of immune cells in a tumor tissue or at the infection site after administration of the anti-SIRPα construct.

43. The method of claim 42, wherein the number of immune cells in a tumor tissue or at the infection site is increased by at least about 5% after administration of the anti-SIRPα construct.


SEQUENCE LISTING (111 sequences)

SEQ ID NO: 1 - protein, 7 aa, synthetic construct
  source 1..7: note = 53.21A9.B11 HC CDR1 (Kabat)
SGRYYWS

SEQ ID NO: 2 - protein, 16 aa, synthetic construct
  source 1..16: note = 53.21A9.B11 HC CDR2 (Kabat)
YIYYSGSTNYNPSLKS

SEQ ID NO: 3 - protein, 10 aa, synthetic construct
  source 1..10: note = 53.21A9.B11 HC CDR3 (Kabat)
AYDWNYFFDY

SEQ ID NO: 4 - protein, 16 aa, synthetic construct
  source 1..16: note = 53.21A9.B11 LC CDR1 (Kabat)
RSSQSLLYSNGYNYLE

SEQ ID NO: 5 - protein, 7 aa, synthetic construct
  source 1..7: note = 53.21A9.B11 LC CDR2 (Kabat)
LGSNRAS

SEQ ID NO: 6 - protein, 9 aa, synthetic construct
  source 1..9: note = 53.21A9.B11 LC CDR3 (Kabat)
MQGLQTPIT

SEQ ID NO: 7 - protein, 120 aa, synthetic construct
  source 1..120: note = 53.21A9.B11 VH amino acid sequence
QVQLQESGPGLVKPSETLSLTCTVSGGSVSSGRYYWSWIRQPPGKGLEWIGYIYYSGSTNYNPSLKSRVTISVDTSKNQFSLKLTSVTAADTAVYYCARAYDWNYFFDYWGQGTLVTVSS

SEQ ID NO: 8 - protein, 112 aa, synthetic construct
  source 1..112: note = 53.21A9.B11 VL amino acid sequence
DIVMTQSPLSLPVTPGEPASISCRSSQSLLYSNGYNYLEWYLQKPGQSPQLLIYLGSNRASGVPDRFSGSGSGTDFTLQISRVEAEDVGVYYCMQGLQTPITFGQGTRLEIK

SEQ ID NO: 9 - protein, 5 aa, synthetic construct
  source 1..5: note = 53.29H9.C7 HC CDR1 (Kabat)
GYYWS

SEQ ID NO: 10 - protein, 16 aa, synthetic construct
  source 1..16: note = 53.29H9.C7 HC CDR2 (Kabat)
EINHSGSTNYNPSLKR

SEQ ID NO: 11 - protein, 12 aa, synthetic construct
  source 1..12: note = 53.29H9.C7 HC CDR3 (Kabat)
GRGYSGYYYFDY

SEQ ID NO: 12 - protein, 12 aa, synthetic construct
  source 1..12: note = 53.29H9.C7 LC CDR1 (Kabat)
RASQSVSSSYLA

SEQ ID NO: 13 - protein, 7 aa, synthetic construct
  source 1..7: note = 53.29H9.C7 LC CDR2 (Kabat)
GASSRAT

SEQ ID NO: 14 - protein, 9 aa, synthetic construct
  source 1..9: note = 53.29H9.C7 LC CDR3 (Kabat)
QQYDSSPLT

SEQ ID NO: 15 - protein, 120 aa, synthetic construct
  source 1..120: note = 53.29H9.C7 VH amino acid sequence
QVQLQQWGAGLLKPSETLSLTCAVYGGSFSGYYWSWIRQPPGKGLDWIGEINHSGSTNYNPSLKRRVTISIDTSKNQFSLKLTSVTAADTAVYYCARGRGYSGYYYFDYWGQGTLVTVSS

SEQ ID NO: 16 - protein, 108 aa, synthetic construct
  source 1..108: note = 53.29H9.C7 VL amino acid sequence
EIVLTQSPGTLSLSPGERATLSCRASQSVSSSYLAWYQQKPGQAPRLLIYGASSRATGIPDRFSRSGSGTDFTLTISRLEPEDFAVYYCQQYDSSPLTFGGGTKVEIK

SEQ ID NO: 17 - protein, 5 aa, synthetic construct
  source 1..5: note = 53.64D2.A7 HC CDR1 (Kabat)
SYDMD

SEQ ID NO: 18 - protein, 16 aa, synthetic construct
  source 1..16: note = 53.64D2.A7 HC CDR2 (Kabat)
GIGIAGDTYYPDSVKG

SEQ ID NO: 19 - protein, 10 aa, synthetic construct
  source 1..10: note = 53.64D2.A7 HC CDR3 (Kabat)
GGGWDGSLDY

SEQ ID NO: 20 - protein, 11 aa, synthetic construct
  source 1..11: note = LC CDR1 of 53.64D2.A7, 54.8A5.A5, 54.58C2.B9.A2, 54.63E11.D9, 54.69B10.E7, 54.75C11.B7, 54.76D9.B6 (Kabat)
RASQDINNYLA

SEQ ID NO: 21 - protein, 7 aa, synthetic construct
  source 1..7: note = LC CDR2 of 53.64D2.A7 and 54.8A5.A5 (Kabat)
TASRLQS

SEQ ID NO: 22 - protein, 9 aa, synthetic construct
  source 1..9: note = LC CDR3 of 53.64D2.A7 and 54.63E11.D9 (Kabat)
QQYNTYPYT

SEQ ID NO: 23 - protein, 118 aa, synthetic construct
  source 1..118: note = 53.64D2.A7 VH amino acid sequence
EVQLVESGGGLVQPGGSLRLSCAASGFTFRSYDMDWVRQATGKGLEWVSGIGIAGDTYYPDSVKGRFTISRENAKNSLYLQMNSLRPGDTAVYYCARGGGWDGSLDYWGQGTLVTVSS

SEQ ID NO: 24 - protein, 107 aa, synthetic construct
  source 1..107: note = 53.64D2.A7 VL amino acid sequence
DIQMTQSPSSLSASVGDRVTITCRASQDINNYLAWFQQKPGRAPKSLIYTASRLQSGVPSKFSGSGSGTDFTLTISSLQPEDFATYYCQQYNTYPYTFGQGTRLEIK

SEQ ID NO: 25 - protein, 5 aa, synthetic construct
  source 1..5: note = HC CDR1 of 54.8A5.A5, 54.14E2.A11, 54.49D12.A6, 54.52H4.B4, 54.54A2.F9, 54.58C2.B9.A2, 54.63E11.D9, 54.69B10.E7, 54.75C11.B7, and 54.76D9.B6 (Kabat)
SYDMH

SEQ ID NO: 26 - protein, 16 aa, synthetic construct
  source 1..16: note = HC CDR2 of 54.8A5.A5, 54.52H4.B4, and 54.54A2.F9 (Kabat)
AIGTAGDTYYPGSVKG

SEQ ID NO: 27 - protein, 10 aa, synthetic construct
  source 1..10: note = HC CDR3 of 54.8A5.A5, 54.14E2.A11, 54.49D12.A6, 54.52H4.B4, 54.54A2.F9, 54.58C2.B9.A2, 54.63E11.D9, 54.69B10.E7, 54.75C11.B7 (Kabat)
GGNWDDAFDI

SEQ ID NO: 28 - protein, 9 aa, synthetic construct
  source 1..9: note = 54.8A5.A5 LC CDR3 (Kabat)
QQYTFYPYT

SEQ ID NO: 29 - protein, 118 aa, synthetic construct
  source 1..118: note = 54.8A5.A5 VH amino acid sequence
EVQLVESGGDLIQPGGSLRLSCAASGFSFSSYDMHWVRQTTGKGLEWVSAIGTAGDTYYPGSVKGRFTISRENAKNSLYLQMNSLRAGDTAVYYCARGGNWDDAFDIWGQGTVVTVSS

SEQ ID NO: 30 - protein, 107 aa, synthetic construct
  source 1..107: note = 54.8A5.A5 VL amino acid sequence
DIQMTQSPSSLSASVGDRVTITCRASQDINNYLAWFQQKPGRAPKSLIFTASRLQSGVPSKFSGSGSGTDFSLTISSLQPEDFATYYCQQYTFYPYTFGQGTKLEIK

SEQ ID NO: 31 - protein, 16 aa, synthetic construct
  source 1..16: note = 54.14E2.A11 HC CDR2 (Kabat)
VIGIAGDTYYTGSVKG

SEQ ID NO: 32 - protein, 11 aa, synthetic construct
  source 1..11: note = LC CDR1 of 54.14E2.A11, 54.49D12.A6, 54.52H4.B4, and 54.54A2.F9 (Kabat)
RASQGINNYLA

SEQ ID NO: 33 - protein, 7 aa, synthetic construct
  source 1..7: note = LC CDR2 of 54.14E2.A11, 54.54A2.F9, 54.63E11.D9, and 54.75C11.B7 (Kabat)
TASSLQS

SEQ ID NO: 34 - protein, 9 aa, synthetic construct
  source 1..9: note = LC CDR3 of 54.14E2.A11, 54.49D12.A6, 54.54A2.F9 (Kabat)
QQYNSYPYT

SEQ ID NO: 35 - protein, 118 aa, synthetic construct
  source 1..118: note = 54.14E2.A11 VH amino acid sequence
EVQLVESGGGLVQPGGSLRLSCAASGFTFSSYDMHWVRQATGKGLEWVSVIGIAGDTYYTGSVKGRFTISRKNAKNSLYLQMNSLRAGDTAIYYCSRGGNWDDAFDIWGQGTMVTVSS

SEQ ID NO: 36 - protein, 107 aa, synthetic construct
  source 1..107: note = 54.14E2.A11 VL amino acid sequence
DIQMTQSPSSLSASVGDRVTITCRASQGINNYLAWFQQKPGKAPKSLIYTASSLQSGVPSKFGGSGSGTDFTLTISSLQPEDFATYYCQQYNSYPYTFGQGTKLEIK

SEQ ID NO: 37 - protein, 16 aa, synthetic construct
  source 1..16: note = 54.49D12.A6 HC CDR2 (Kabat)
VIGTAGDTYFPGSVKG

SEQ ID NO: 38 - protein, 7 aa, synthetic construct
  source 1..7: note = 54.49D12.A6 LC CDR2 (Kabat)
TVSRLQS

SEQ ID NO: 39 - protein, 118 aa, synthetic construct
  source 1..118: note = 54.49D12.A6 VH amino acid sequence
EVQLVESGGGLVQPGGSLRLSCAASGFTFSSYDMHWVRQATGKGLEWVSVIGTAGDTYFPGSVKGRFTISRENAKNSLYLQMNSLRAGDTAVYYCVRGGNWDDAFDIWGQGTMVTVSS

SEQ ID NO: 40 - protein, 107 aa, synthetic construct
  source 1..107: note = 54.49D12.A6 VL amino acid sequence
DIQMTQSPSSLSASVGDRVTITCRASQGINNYLAWFQQKPGKAPKSLIYTVSRLQSGVPSKFSGSGSGTDFTLTISSLQPEDFATYYCQQYNSYPYTFGQGTKLEIK

SEQ ID NO: 41 - protein, 7 aa, synthetic construct
  source 1..7: note = LC CDR2 of 54.52H4.B4, 54.58C2.B9.A2, 54.69B10.E7, and 54.76D9.B6 (Kabat)
TTSSLQS

SEQ ID NO: 42 - protein, 9 aa, synthetic construct
  source 1..9: note = 54.52H4.B4 LC CDR3 (Kabat)
QQYISYPYT

SEQ ID NO: 43 - protein, 118 aa, synthetic construct
  source 1..118: note = 54.52H4.B4 VH amino acid sequence
EVQLVESGGGLVQPGGSLRLSCAASGFTFSSYDMHWVRQGTEKGLEWVSAIGTAGDTYYPGSVKGRFTISRENAKNSLFLQMNSLRAGDTAVYYCARGGNWDDAFDIWGQGTMVTVSS

SEQ ID NO: 44 - protein, 107 aa, synthetic construct
  source 1..107: note = 54.52H4.B4 VL amino acid sequence
DIQMTQSPSSLSASVGDRVTITCRASQGINNYLAWFQQKPGKAPKSLIYTTSSLQSGVPSKFSGSGSGTDYTLTISSLQPEDFATYYCQQYISYPYTFGQGTKLEIK

SEQ ID NO: 45 - protein, 118 aa, synthetic construct
  source 1..118: note = 54.54A2.F9 VH amino acid sequence
EVQLVESGGGLVQPGGSLRLSCAASGFTFSSYDMHWVRQGTGKGLEWVSAIGTAGDTYYPGSVKGRFTISRENAKNSLFLQMNSLRAGDTAVYYCARGGNWDDAFDIWGQGTMVTVSS

SEQ ID NO: 46 - protein, 107 aa, synthetic construct
  source 1..107: note = 54.54A2.F9 VL amino acid sequence
DIQMTQSPSSLSASVGDRVTITCRASQGINNYLAWFQQKPGKAPKSLIYTASSLQSGVPSKFSGSGSGTDYTLTISSLQPEDFATYYCQQYNSYPYTFGQGTKLEIK

SEQ ID NO: 47 - protein, 16 aa, synthetic construct
  source 1..16: note = HC CDR2 of 54.58C2.B9.A2, 54.63E11.D9, and 54.76D9.B6 (Kabat)
VIGISGDTYYPGSVKG

SEQ ID NO: 48 - protein, 9 aa, synthetic construct
  source 1..9: note = LC CDR3 of 54.58C2.B9.A2, 54.69B10.E7, and 54.76D9.B6 (Kabat)
QQYVSYPYT

SEQ ID NO: 49 - protein, 118 aa, synthetic construct
  source 1..118: note = VH amino acid sequence of 54.58C2.B9.A2, and 54.63E11.D9
EVQLVESGGGLVQPGGSLRLSCAASGFTFSSYDMHWVRQATGKGLEWVSVIGISGDTYYPGSVKGRFTISRENAKNSLYLQMNSLRAGDTAVYYCARGGNWDDAFDIWGQGTMVTVSS

SEQ ID NO: 50 - protein, 107 aa, synthetic construct
  source 1..107: note = VL amino acid sequence of 54.58C2.B9.A2, 54.69B10.E7, and 54.76D9.B6
DIQMTQSPSSLSASVGDRVTITCRASQDINNYLAWFQQKPGKAPKSLIYTTSSLQSGVPSKFSGSGSGTDFTLTISSLQPEDFATYYCQQYVSYPYTFGQGTKLEIK

SEQ ID NO: 51 - protein, 118 aa, synthetic construct; identical to SEQ ID NO: 49
  source 1..118: note = VH amino acid sequence of 54.63E11.D9 and 54.58C2.B9.A2

SEQ ID NO: 52 - protein, 107 aa, synthetic construct
  source 1..107: note = 54.63E11.D9 VL amino acid sequence
DIQMTQSPSSLSASVGDRVTITCRASQDINNYLAWFQQKPGKAPKSQIYTASSLQSGVPSKFSGSGSGTDFTLTISSLQPEDFATYYCQQYNTYPYTFGQGTKLEIK

SEQ ID NO: 53 - protein, 16 aa, synthetic construct
  source 1..16: note = HC CDR2 of 54.69B10.E7 and 54.75C11.B7 (Kabat)
VIGIAGDTYYAGSVKG

SEQ ID NO: 54 - protein, 118 aa, synthetic construct
  source 1..118: note = VH amino acid sequence of 54.69B10.E7 and 54.75C11.B7
EVQLVESGGGLVQPGGSLRLSCAASGFTFSSYDMHWVRQTTGKGLEWVSVIGIAGDTYYAGSVKGRFTISRENAKSSLYLQMNSLRAGDTAVYYCARGGNWDDAFDIWGQGTMVTVSS

SEQ ID NO: 55 - protein, 107 aa, synthetic construct; identical to SEQ ID NO: 50
  source 1..107: note = VL amino acid sequence of 54.69B10.E7, 54.58C2.B9.A2, and 54.76D9.B6

SEQ ID NO: 56 - protein, 9 aa, synthetic construct
  source 1..9: note = 54.75C11.B7 LC CDR3 (Kabat)
QQYSTYPYT

SEQ ID NO: 57 - protein, 118 aa, synthetic construct; identical to SEQ ID NO: 54
  source 1..118: note = VH amino acid sequence of 54.75C11.B7 and 54.69B10.E7

SEQ ID NO: 58 - protein, 107 aa, synthetic construct
  source 1..107: note = 54.75C11.B7 VL amino acid sequence
DIQMTQSPSSLSASVGDRVTITCRASQDINNYLAWFQQKPGKAPKSQIYTASSLQSGVPSKFSGSGSGTDFTLTISSLQPEDFATYYCQQYSTYPYTFGQGTKLEIK

SEQ ID NO: 59 - protein, 10 aa, synthetic construct
  source 1..10: note = 54.76D9.B6 HC CDR3 (Kabat)
GGNWDDALDI

SEQ ID NO: 60 - protein, 118 aa, synthetic construct
  source 1..118: note = VH amino acid sequence of 54.76D9.B6 and 54.75C11.B7
EVQLVESGGGLVQPGGSLRLSCAASGFTFSSYDMHWVRQATGKGLEWVSVIGISGDTYYPGSVKGRFTISRENAKNSLHLQMNSLRAGDTAVYYCARGGNWDDALDIWGQGTMVTVSS

SEQ ID NO: 61 - protein, 107 aa, synthetic construct; identical to SEQ ID NO: 50
  source 1..107: note = VL amino acid sequence of 54.76D9.B6, 54.58C2.B9.A2, and 54.69B10.E7

SEQ ID NO: 62 - protein, 5 aa, synthetic construct
  source 1..5: note = Group C consensus sequence - CDRH1
  VARIANT 5: note = X is D or H
SYDMX

SEQ ID NO: 63 - protein, 16 aa, synthetic construct
  source 1..16: note = Group C consensus sequence - CDRH2
  VARIANT 11: note = X is A, P, or T
  VARIANT 10: note = X is Y or F
  VARIANT 5: note = X is A or S
  VARIANT 4: note = X is I or T
  VARIANT 1: note = X is A or V
XIGXXGDTYXXGSVKG

SEQ ID NO: 64 - protein, 10 aa, synthetic construct
  source 1..10: note = Group C consensus sequence - CDRH3
  VARIANT 8: note = X is D or L
GGNWDDAXDI

SEQ ID NO: 65 - protein, 11 aa, synthetic construct
  source 1..11: note = Group C consensus sequence - CDRL1
  VARIANT 5: note = X is D or G
RASQXINNYLA

SEQ ID NO: 66 - protein, 7 aa, synthetic construct
  source 1..7: note = Group C consensus sequence - CDRL2
  VARIANT 4: note = X is R or S
  VARIANT 2: note = X is A, V or T
TXSXLQS

SEQ ID NO: 67 - protein, 9 aa, synthetic construct
  source 1..9: note = Group C consensus sequence - CDRL3
  VARIANT 5: note = X is F, T or S
  VARIANT 4: note = X is I, N, T, V or S
QQYXXYPYT

SEQ ID NO: 68 - skipped

SEQ ID NO: 69 - protein, 16 aa, synthetic construct
  source 1..16: note = Linker; note = up to 7 copies of GS can be deleted
GSGSGSGSGSGSGSGS

SEQ ID NO: 70 - protein, 40 aa, synthetic construct
  source 1..40: note = Linker; note = up to 7 copies of GSGGS can be deleted
GSGGSGSGGSGSGGSGSGGSGSGGSGSGGSGSGGSGSGGS

SEQ ID NO: 71 - protein, 40 aa, synthetic construct
  source 1..40: note = Linker; note = up to 7 copies of GGGGS can be deleted
GGGGSGGGGSGGGGSGGGGSGGGGSGGGGSGGGGSGGGGS

SEQ ID NO: 72 - protein, 32 aa, synthetic construct
  source 1..32: note = Linker; note = up to 7 copies of GGGS can be deleted
GGGSGGGSGGGSGGGSGGGSGGGSGGGSGGGS

SEQ ID NO: 73 - protein, 15 aa, synthetic construct
  source 1..15: note = Linker
GGGGSGGGGSGGGGS

SEQ ID NO: 74 - protein, 30 aa, synthetic construct
  source 1..30: note = Linker
GGGGSGGGGSGGGGSGGGGSGGGGSGGGGS

SEQ ID NO: 75 - protein, 45 aa, synthetic construct
  source 1..45: note = Linker; note = up to 2 copies of GSTSGSGKPGSGEGS can be deleted
GSTSGSGKPGSGEGSGSTSGSGKPGSGEGSGSTSGSGKPGSGEGS

SEQ ID NO: 76 - protein, 32 aa, synthetic construct; identical to SEQ ID NO: 72
  source 1..32: note = Linker; note = up to 7 copies of GGGS can be deleted

SEQ ID NO: 77 - protein, 504 aa, Homo sapiens
  source 1..504: note = Human SIRPalpha UniProtKB/Swiss-Prot: P78324
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 78 - protein, 5 aa, synthetic construct
  source 1..5: note = 49.56C11.C1 HC CDR1 (Kabat)
NHAMS

SEQ ID NO: 79 - protein, 17 aa, synthetic construct
  source 1..17: note = 49.56C11.C1 HC CDR2 (Kabat)
TFGSGGSYTYYLDSVKG

SEQ ID NO: 80 - protein, 9 aa, synthetic construct
  source 1..9: note = HC CDR3 of 49.56C11.C1 and 49.64D3.A6 (Kabat)
SGWDGWFAY

SEQ ID NO: 81 - protein, 17 aa, synthetic construct
  source 1..17: note = 49.56C11.C1 LC CDR1 (Kabat)
KSSQSLLNSNNQKNYLA

SEQ ID NO: 82 - protein, 7 aa, synthetic construct
  source 1..7: note = LC CDR2 of 49.56C11.C1 and 49.64D3.A6 (Kabat)
FASTRES

SEQ ID NO: 83 - protein, 10 aa, synthetic construct
  source 1..10: note = 49.56C11.C1 LC CDR3 (Kabat)
QQHYSTLPWT

SEQ ID NO: 84 - protein, 118 aa, synthetic construct
  source 1..118: note = 49.56C11.C1 VH amino acid sequence
EVMLVESGGGLVKPGGSLKLTCAASGFTFSNHAMSWVRQTPEKRLEWVATFGSGGSYTYYLDSVKGRFTVSRDNAKNTLYLQMSSLRSEDTAMYYCSRSGWDGWFAYWGQGTLVTVSA

SEQ ID NO: 85 - protein, 114 aa, synthetic construct
  source 1..114: note = 49.56C11.C1 VL amino acid sequence
DIVMTQSPSSLAMSIGQKVTMNCKSSQSLLNSNNQKNYLAWYQQKPGQSPKLLIYFASTRESGVPDRFIGSGSGTDFTLTISSVQAEDLANYFCQQHYSTLPWTFGGGTKLEIK

SEQ ID NO: 86 - protein, 5 aa, synthetic construct
  source 1..5: note = 49.64D3.A6 HC CDR1 (Kabat)
NYAMS

SEQ ID NO: 87 - protein, 17 aa, synthetic construct
  source 1..17: note = 49.64D3.A6 HC CDR2 (Kabat)
TFSSGGSYTYYQDSVKG

SEQ ID NO: 88 - protein, 17 aa, synthetic construct
  source 1..17: note = 49.64D3.A6 LC CDR1 (Kabat)
KSSQSLLNSSNQKNYLA

SEQ ID NO: 89 - protein, 10 aa, synthetic construct
  source 1..10: note = 49.64D3.A6 LC CDR3 (Kabat)
QQHCSTLPWT

SEQ ID NO: 90 - protein, 118 aa, synthetic construct
  source 1..118: note = 49.64D3.A6 VH amino acid sequence
EVMLVESGGGLVKPGGSLKLTCAASGFIFSNYAMSWVRQTPEKRLEWVATFSSGGSYTYYQDSVKGRFTVSRDNAKNTLYLQMSSLRSEDTAMYYCARSGWDGWFAYWGQGTLVTVSA

SEQ ID NO: 91 - protein, 114 aa, synthetic construct
  source 1..114: note = 49.64D3.A6 VL amino acid sequence
DIVMTQSPSSLAMSVGQKVTMSCKSSQSLLNSSNQKNYLAWYQQKPGQSPKLLVYFASTRESGVPDRFIGSGSGTDFTLTISSVQAEDLANYFCQQHCSTLPWTFGGGTKLEIK

SEQ ID NO: 92 - protein, 5 aa, synthetic construct
  source 1..5: note = 49.91C2.A9 HC CDR1 (Kabat)
RYWMS

SEQ ID NO: 93 - protein, 17 aa, synthetic construct
  source 1..17: note = 49.91C2.A9 HC CDR2 (Kabat)
EINPDSSTINYTPSLKD

SEQ ID NO: 94 - protein, 12 aa, synthetic construct
  source 1..12: note = 49.91C2.A9 HC CDR3 (Kabat)
SFYGSSYWYFDV

SEQ ID NO: 95 - protein, 15 aa, synthetic construct
  source 1..15: note = 49.91C2.A9 LC CDR1 (Kabat)
RASESVDNYGISFMN

SEQ ID NO: 96 - protein, 7 aa, synthetic construct
  source 1..7: note = 49.91C2.A9 LC CDR2 (Kabat)
AASNQGS

SEQ ID NO: 97 - protein, 9 aa, synthetic construct
  source 1..9: note = 49.91C2.A9 LC CDR3 (Kabat)
QQSKEVPYT

SEQ ID NO: 98 - protein, 121 aa, synthetic construct
  source 1..121: note = 49.91C2.A9 VH amino acid sequence
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCATSFYGSSYWYFDVWGAGTTVTVSS

SEQ ID NO: 99 - protein, 111 aa, synthetic construct
  source 1..111: note = 49.91C2.A9 VL amino acid sequence
DIVLTQSPASLAVSLGQRATISCRASESVDNYGISFMNWFQQKPGQPPKVLIYAASNQGSGVPARFSGSGSGTDFSLNIHPMEEDDTAMYFCQQSKEVPYTFGGGTKLEIK

SEQ ID NO: 100 - protein, 5 aa, synthetic construct
  source 1..5: note = 56.16A1.H11 HC CDR1 (Kabat)
NYDMA

SEQ ID NO: 101 - protein, 17 aa, synthetic construct
  source 1..17: note = 56.16A1.H11 HC CDR2 (Kabat)
SISYGGSRIYYRDSVKG

SEQ ID NO: 102 - protein, 14 aa, synthetic construct
  source 1..14: note = 56.16A1.H11 HC CDR3 (Kabat)
DYGYNPSYYWYFDF

SEQ ID NO: 103 - protein, 11 aa, synthetic construct
  source 1..11: note = 56.16A1.H11 LC CDR1 (Kabat)
RASQGISNYLN

SEQ ID NO: 104 - protein, 7 aa, synthetic construct
  source 1..7: note = 56.16A1.H11 LC CDR2 (Kabat)
YTSNLQS

SEQ ID NO: 105 - protein, 9 aa, synthetic construct
  source 1..9: note = 56.16A1.H11 LC CDR3 (Kabat)
QQYDSSPYT

SEQ ID NO: 106 - protein, 123 aa, synthetic construct
  source 1..123: note = 56.16A1.H11 VH amino acid sequence
EVQLVESGGGLVQPGRSMKLSCAASGFIFTNYDMAWVRQAPTKGLEWVASISYGGSRIYYRDSVKGRFTISRDDAKSTLYLQMDSLRSEDTATYYCTTDYGYNPSYYWYFDFWGPGTMVTVSS

SEQ ID NO: 107 - protein, 108 aa, synthetic construct
  source 1..108: note = 56.16A1.H11 VL amino acid sequence
DIQMTQTPSSMPASLGERVTISCRASQGISNYLNWYQQKPDGTIKPLIYYTSNLQSGVPSRFSGSGSGTDYSLTISSLEPEDFAMYYCQQYDSSPYTFGAGTKLELKR

SEQ ID NO: 108 - protein, 5 aa, synthetic construct
  source 1..5: note = Group D consensus sequence - CDRH1
  VARIANT 2: note = X is H or Y
NXAMS

SEQ ID NO: 109 - protein, 17 aa, synthetic construct
  source 1..17: note = Group D consensus sequence - CDRH2
  VARIANT 3: note = X is G or S
  VARIANT 12: note = X is L or Q
TFXSGGSYTYYXDSVKG

SEQ ID NO: 110 - protein, 17 aa, synthetic construct
  source 1..17: note = Group D consensus sequence - CDRL1
  VARIANT 10: note = X is N or S
KSSQSLLNSXNQKNYLA

SEQ ID NO: 111 - protein, 10 aa, synthetic construct
  source 1..10: note = Group D consensus sequence - CDRL3
  VARIANT 4: note = X is Y or C
QQHXSTLPWT